(19) United States
(12) Patent Application Publication
(10) Pub. No.: US 2025/0229262 A1
(43) Pub. Date: Jul. 17, 2025

(54) CHIRAL SPIROCYCLIC COMPOUNDS AND METHODS OF PREPARATION AND USE THEREOF

(71) Applicant: The Hong Kong University of Science and Technology, Hong Kong (CN)

(72) Inventors: Jianwei SUN, Hong Kong (CN); Ronghua ZHANG, Hong Kong (CN); Jingyi WANG, Hong Kong (CN); Youshao TU, Hong Kong (CN); Chenxiao QIAN, Shanghai (CN); Qiang DAI, Hong Kong (CN); Yu BAO, Hong Kong (CN)

(21) Appl. No.: 19/014,358

(22) Filed: Jan. 9, 2025

Related U.S. Application Data
(60) Provisional application No. 63/622,039, filed on Jan. 17, 2024.

(51) Int. Cl. B01J 31/24; B01J 31/18; C07C 275/30; C07C 335/18; C07D 209/12; C07D 263/12; C07F 9/54; C07F 9/572; C07F 9/6574

(52) U.S. Cl. CPC B01J 31/249; B01J 31/182; B01J 31/187; B01J 31/189; C07C 275/30; C07C 335/18; C07D 209/12; C07D 263/12; C07F 9/5442; C07F 9/5728; C07F 9/65748; B01J 2231/328; B01J 2231/34; B01J 2231/4288; B01J 2231/44; B01J 2231/645; B01J 2531/004; B01J 2531/0261; B01J 2531/822; B01J 2531/824; B01J 2531/827; C07C 2603/54

(57) ABSTRACT

Spirocyclic compounds, including chiral spiro diamine, chiral spiro amino naphthol, chiral spiro bis(indole), chiral spiro diaryl diol, chiral spiro diaryl diamine, chiral spiro amino naphthol, chiral spiro diaryl diindole, and chiral spiro phospholane useful as chiral ligands and chiral organocatalysts and methods of preparation and methods of use thereof. Owing to the molecular shape and three-dimensional orientation, the chiral diamine and chiral amino naphthol molecules provide a skeleton for use as ligands and organocatalysts.

### CROSS-REFERENCE TO RELATED APPLICATIONS

The present application claims priority from U.S. Provisional Patent Application No. 63/622,039, filed on Jan. 17, 2024, which is hereby incorporated by reference in its entirety.

### TECHNICAL FIELD

The present disclosure relates to compounds useful in the preparation of enantioselective catalysts and methods of preparation and use thereof.

### BACKGROUND

Functionalized axially chiral biaryl skeletons such as 1,1′-bi-2-naphthol (BINOL), 1,1′-binaphthyl-2,2′-diamine (BINAM), have been widely used as privilege scaffolds for developing chiral ligands and catalysts. While the asymmetric synthesis of BINOL has been well developed in the past decades, BINAM, with a similar axially chiral skeleton, remains largely unexplored concerning its application in asymmetric synthesis. Both lengthy synthesis routines and high prices probably limited its development. 1,1′-Spirobiindane-7,7′-diamine prepared from SPINOL, which also owns a privileged spiro backbone, is highly under-developed in asymmetric catalysis, probably due to the lengthy synthetic routine and expensive optically pure starting material. Thus, it is not a surprise that such a biaryl amine skeleton is hardly explored in the development of ligands and catalysts. Therefore, the unknown catalytic asymmetric synthesis of biaryl amine with spiro skeleton is highly demanded but challenging. Axially chiral biaryl amino naphthols have recently been extensively implemented as chiral ligands or organocatalysts. The NOBIN (2-amino-2′-hydroxy-1,1′-binaphthyl) structure is present in a multitude of highly prized biaryl atropisomeric catalysts and ligands for chirality induction in asymmetric catalysis. In contrast, biaryl amino naphthols based on spiro skeleton are unknown, mainly due to a lack of asymmetric synthesis. Spiro phospholane has the same problem and is now widely used in SPINOL-derived spiro phospholane (SITCP), which is extremely sensitive to oxygen and requires strict oxygen-free preparation, so synthesis is expensive. Besides, the development of effective chiral catalysts, especially ligands having novel chiral backbones, is still an important task around asymmetric catalysis. Many reactions still lack effective chiral ligands, and the enantioselectivities in many reactions are substrate-dependent. The development of effective chiral catalysts, especially ligands having novel chiral backbones, is still an important task in asymmetric catalysis.

There thus exists a need for improved chiral spirocyclic compounds that address or overcome at least some of the disadvantages in the art described above.

### SUMMARY

In a first aspect, provided herein is a compound of Formula 1, 2, 3, or 4:

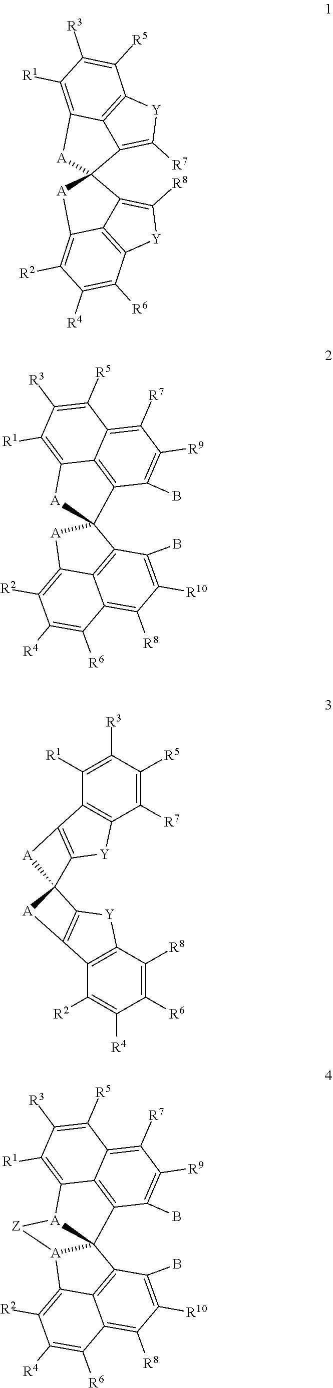

or a conjugate salt thereof, wherein:
A is —(CR₂)ₙ—, wherein n for each instance is a whole number selected from 1-5 and R for each instance is independently hydrogen, alkyl, cycloalkyl, aryl, aralkyl, or a covalent bond to Z;
each B is independently —CH₂OR²⁰, —CH₂NR²⁰₂, —OR²⁰, —NR²⁰₂, —PR²⁰₂, —OP(OR²⁰)₂, —NHPR²⁰₂, —NHP(OR²⁰)₂, —NH(C═S)NHR²⁰, or a moiety represented by:

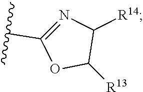

or
two instances of B taken together form a moiety having a structure selected from the group consisting of:

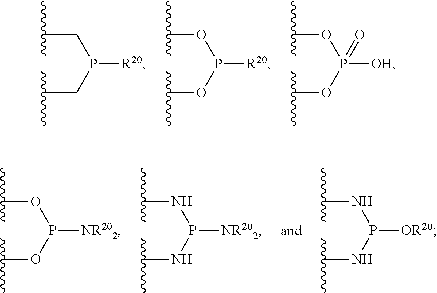

each Y is independently selected from the group consisting of: —O—, —S—, —(NR²⁰)—, and —(NPR²⁰₂)—;
Z is —(CR₂)ₙ—, wherein n for each instance is independently a whole number selected from 1-5 and R for each instance is independently hydrogen, alkyl, cycloalkyl, aryl, or aralkyl;
each of R¹, R², R³, R⁴, R⁵, R⁶, R⁷, R⁸, R⁹, and R¹⁰ is independently selected from the group consisting of hydrogen, alkyl, cycloalkyl, heterocycloalkyl, aralkyl, aryl, heteroaryl, halide, trialkylsilane, and triarylsilane; or
each of R⁷ and R⁸ is independently —CH₂OR²⁰, —CH₂NR²⁰₂, —OR²⁰, —NR²⁰₂, —PR²⁰₂, —OP(OR²⁰)₂, —NHPR²⁰₂, —NHP(OR²⁰)₂, or a moiety represented by:

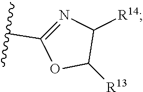

or
R⁷ and R⁸ is taken together form a moiety having a structure selected from the group consisting of:

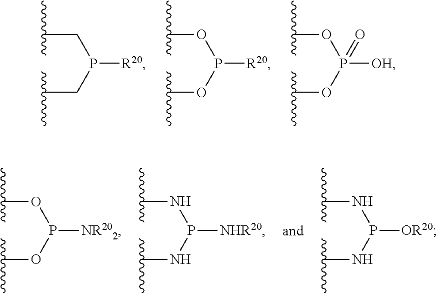

each of R¹³ and R¹⁴ is independently, hydrogen, alkyl, cycloalkyl, aryl, or aralkyl; and
R²⁰ for each instance is independently hydrogen, alkyl, cycloalkyl, heterocycloalkyl, aryl, aralkyl, heteroaryl, or —CHPhCH₂PPh₂; or two instances of R²⁰ taken together form 5-7 membered heterocyclic group.

In certain embodiments, the compound is substantially enantiomerically pure.

In certain embodiments, n is 1 or 2.

In certain embodiments, each of R¹, R², R³, R⁴, R⁵, and R⁶ is hydrogen.

In certain embodiments, each of R⁷, R⁸, R⁹, and R¹⁰ is hydrogen, alkyl, cycloalkyl, or aryl; or each of R⁷ and R⁸ is independently —CH₂OR²⁰, —CH₂NR²⁰₂, —OR²⁰, —NR²⁰₂, —PR²⁰₂, —OP(OR²⁰)₂, —NHPR²⁰₂, or —NHP(OR²⁰)₂.

In certain embodiments, each of R¹, R², R³, R⁴, R⁵, R⁶, R⁷, R⁸, R⁹, and R¹⁰ is hydrogen.

In certain embodiments, the compound has Formula 2 and each B is independently —CH₂OR²⁰, —CH₂NR²⁰₂, —OR²⁰, —NR²⁰₂, —PR²⁰₂, —NHPR²⁰₂, —OP(OR²⁰)₂, —NH(C═S)NHR²⁰, or a moiety represented by:

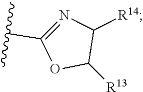

or
two instances of B taken together form a moiety represented by:

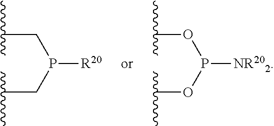

In certain embodiments, the compound has Formula 2 and each B is independently —NHPR²⁰₂, —OP(OR²⁰)₂, —NH(C═S)NHCHPhCH₂PPh₂, —OR²⁰, or a moiety represented by:

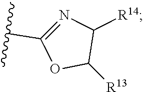

or
two instances of B taken together form a moiety represented by:

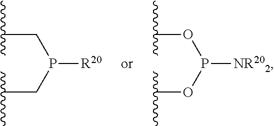

wherein R²⁰ for each instance is independently alkyl, cycloalkyl, or aryl.

In certain embodiments, the compound has Formula 1 or 3 and each Y is —(NPR²⁰₂)—.

In certain embodiments, R²⁰ is alkyl, cycloalkyl, or aryl.

In certain embodiments, the compound has Formula 1 or 3; each Y is —(NPR²⁰₂)—; and R²⁰ is alkyl, cycloalkyl, or aryl; or
the compound has Formula 2; each B is independently —NHPR²⁰₂, —OP(OR²⁰)₂, —NH(C═S)NHCHPhCH₂PPh₂, —OR²⁰, or a moiety represented by:

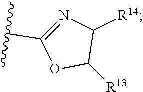

or
two instances of B taken together form a moiety represented by:

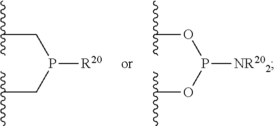

and
R²⁰ for each instance is independently alkyl, cycloalkyl, aryl, or heteroaryl; or two instances of R²⁰ taken together form 5-7 membered heterocyclic group.

In certain embodiments, each of R¹, R², R³, R⁴, R⁵, R⁶, R⁷, R⁸, R⁹, and R¹⁰ is independently selected from the group consisting of hydrogen, alkyl, cycloalkyl, aralkyl, aryl, and heteroaryl.

In certain embodiments, the compound has Formula 1 or 3; each of R¹, R², R³, R⁴, R⁵, R⁶, R⁷, and R⁸ is hydrogen; each Y is —(NPR²⁰₂)—; and R²⁰ is alkyl, cycloalkyl, or aryl; or
the compound has Formula 2; each of R¹, R², R³, R⁴, R⁵, R⁶, R⁷, R⁸, R⁹, and R¹⁰ is hydrogen; each B is independently —NHPR²⁰₂, —OP(OR²⁰)₂, —NH(C═S)NHCHPhCH₂PPh₂, —OR²⁰, or a moiety represented by:

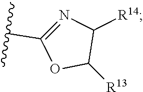

or
two instances of B taken together form a moiety represented by:

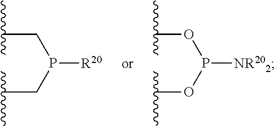

and
R²⁰ for each instance is independently alkyl, cycloalkyl, aryl, or heteroaryl; or two instances of R²⁰ taken together form 5-7 membered heterocyclic group.

In certain embodiments, the compound is selected from the group consisting of:

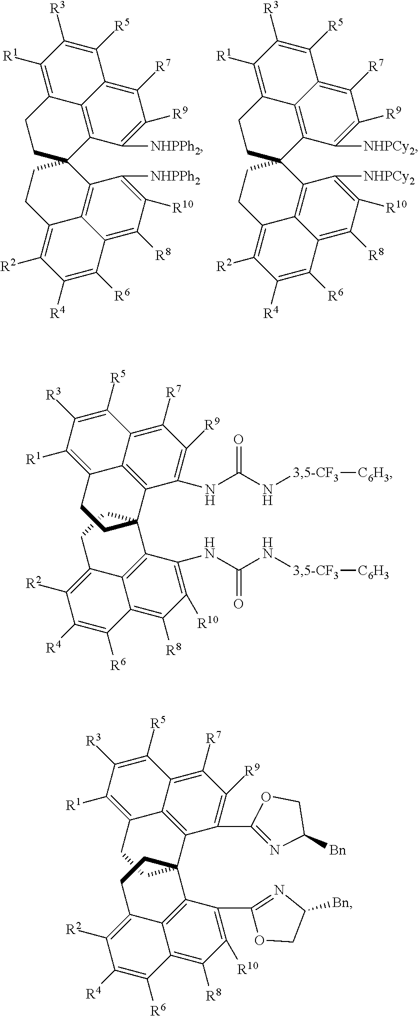

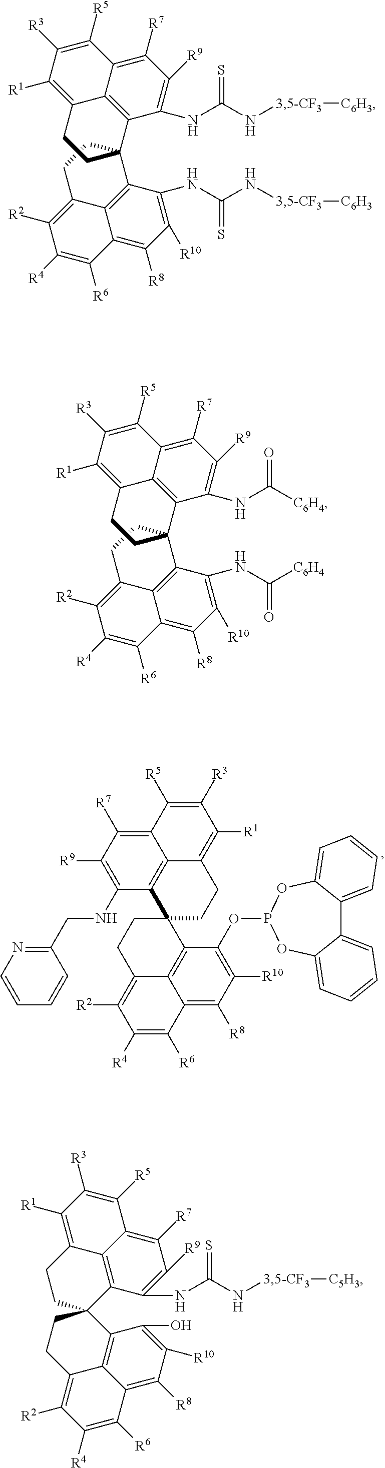

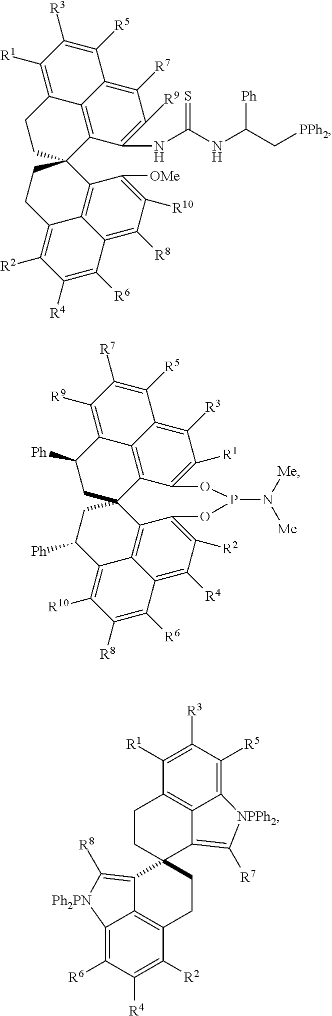

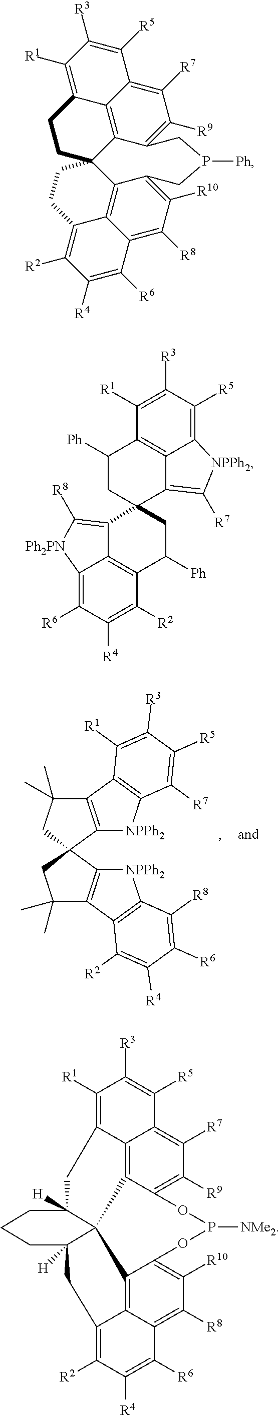

wherein each of R¹, R², R³, R⁴, R⁵, R⁶, R⁷, R⁸, R⁹, and R¹⁰ is independently selected from the group consisting of hydrogen, alkyl, cycloalkyl, aralkyl, aryl, and heteroaryl.

In certain embodiments, the compound is selected from the group consisting of:

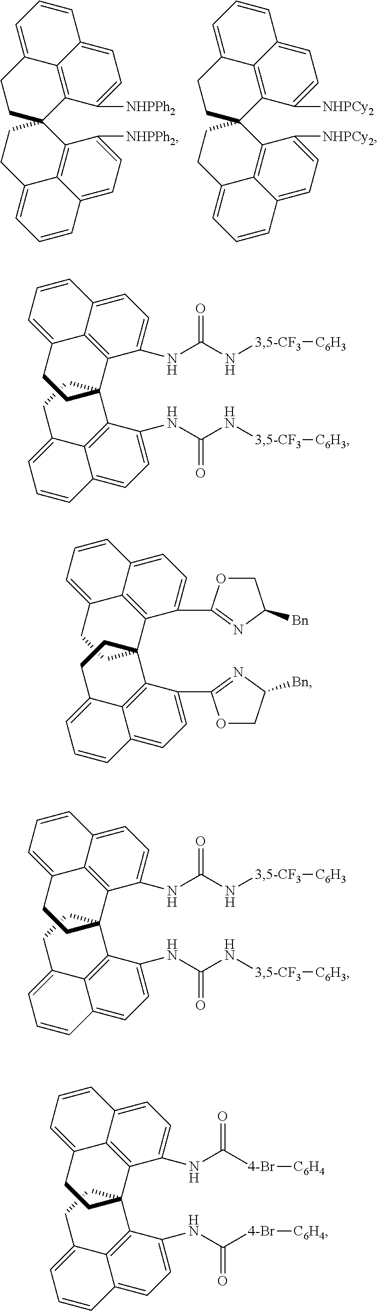

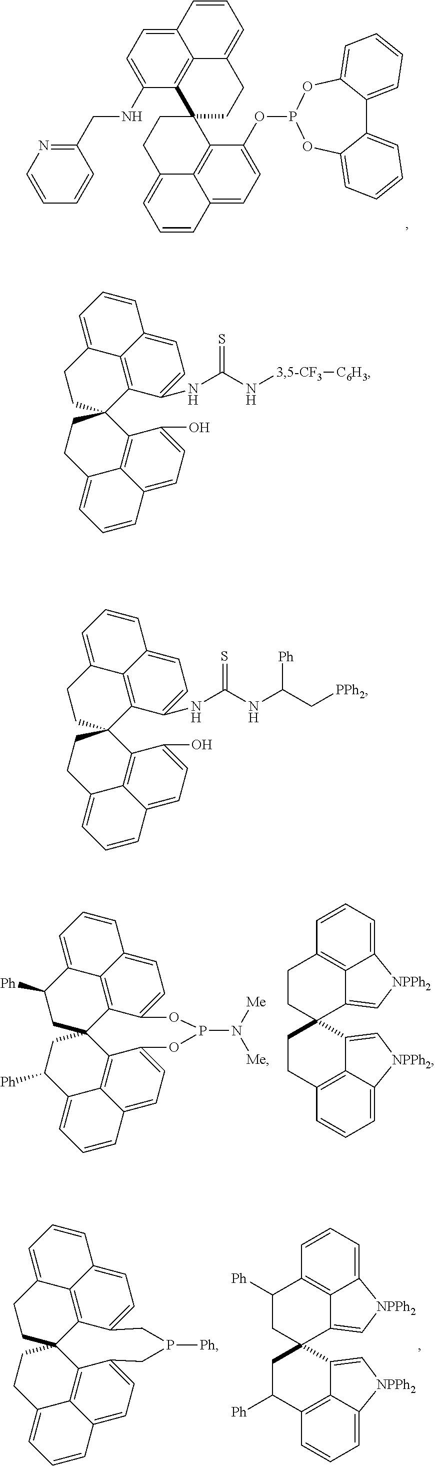

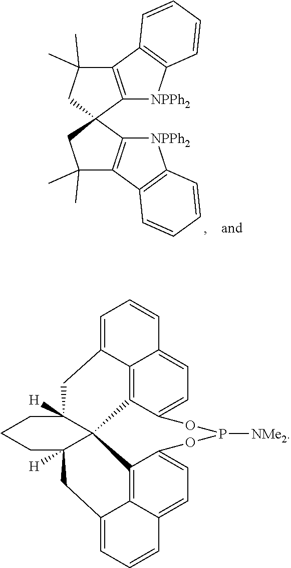

In a second aspect, provided herein is a catalyst comprising the compound of described herein and a metal.

In certain embodiments, the metal is copper, palladium, platinum, iridium, ruthenium, or rhodium.

In a third aspect, provided herein is a method of preparing the compound described therein or a synthon thereof, the method comprising: contacting a compound of Formula 5 or 6:

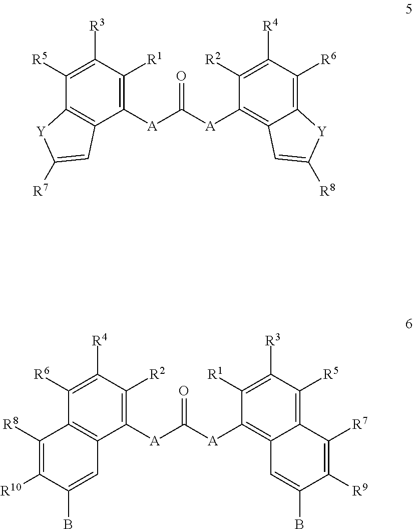

wherein
A is —(CR₂)ₙ—, wherein n is a whole number selected from 1-5 and R for each instance is independently hydrogen, alkyl, cycloalkyl, aryl, or aralkyl;
each B is independently —OR²⁰ or —NR²⁰₂; and
each of R¹, R², R³, R⁴, R⁵, R⁶, R⁷, R⁸, R⁹, and R¹⁰ is independently selected from the group consisting of hydrogen, alkyl, cycloalkyl, heterocycloalkyl, aralkyl, aryl, heteroaryl, halide, trialkylsilane, and triarylsilane; and
R²⁰ for each instance is independently hydrogen, alkyl, cycloalkyl, heterocycloalkyl, aryl, aralkyl, heteroaryl, or —CHPhCH₂PPh₂; or two instances of R²⁰ taken together form 5-7 membered heterocyclic group;
with a phosphoric acid selected from the group consisting of:

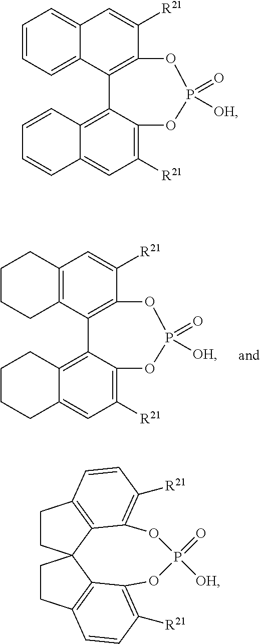

R²¹ is aryl or triarylsilyl thereby forming the compound or synthon thereof.

In certain embodiments, the phosphoric acid is substantially enantiomerically pure.

In certain embodiments, the phosphoric acid is:

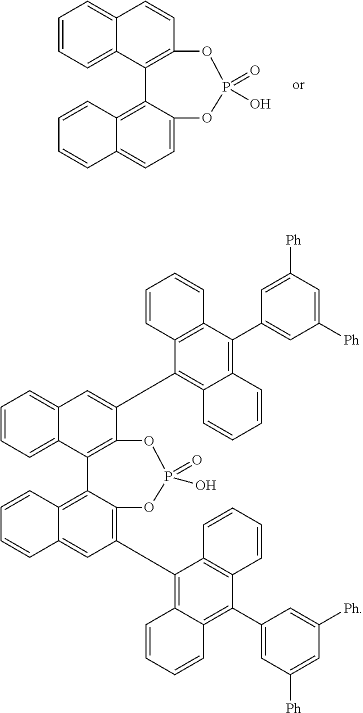 [SMILES: O=P1(O)Oc2ccc3ccccc3c2-c2c(ccc3ccccc23)O1 ; O=P1(O)Oc2c(-c3c4ccccc4c(-c4cc(-c5ccccc5)cc(-c5ccccc5)c4)c4ccccc34)cc3ccccc3c2-c2c(c(-c3c4ccccc4c(-c4cc(-c5ccccc5)cc(-c5ccccc5)c4)c4ccccc34)cc3ccccc23)O1]

### DETAILED DESCRIPTION

### Definitions

The following terms shall be used to describe the present invention. In the absence of a specific definition set forth herein, the terms used to describe the present invention shall be given their common meaning as understood by those of ordinary skill in the art.

Throughout the present disclosure, unless the context requires otherwise, the word “comprise” or variations such as “comprises” or “comprising”, will be understood to imply the inclusion of a stated integer or group of integers but not the exclusion of any other integer or group of integers. It is also noted that in this disclosure and particularly in the claims and/or paragraphs, terms such as “comprises”, “comprised”, “comprising” and the like can have the meaning attributed to it in U.S. Patent law; e.g., they can mean “includes”, “included”, “including”, and the like; and that terms such as “consisting essentially of” and “consists essentially of” have the meaning ascribed to them in U.S. Patent law, e.g., they allow for elements not explicitly recited, but exclude elements that are found in the prior art or that affect a basic or novel characteristic of the present invention.

Furthermore, throughout the present disclosure and claims, unless the context requires otherwise, the word “include” or variations such as “includes” or “including”, will be understood to imply the inclusion of a stated integer or group of integers, but not the exclusion of any other integer or group of integers.

The use of the singular herein includes the plural (and vice versa) unless specifically stated otherwise. In addition, where the use of the term “about” is before a quantitative value, the present teachings also include the specific quantitative value itself, unless specifically stated otherwise. As used herein, the term “about” refers to a ±10%, ±7%, ±5%, ±3%, ±1%, or ±0% variation from the nominal value unless otherwise indicated or inferred.

As used herein, unless otherwise indicated, the term “halo” or “halide” includes fluoro, chloro, bromo or iodo.

As used herein, “alkyl” refers to a straight-chain or branched saturated hydrocarbon group. Examples of alkyl groups include methyl-, ethyl-, propyl (e.g., n-propyl and isopropyl), butyl (e.g., n-butyl, iso-butyl, sec-butyl, tert-butyl), pentyl groups (e.g., 1-methylbutyl, 2-methylbutyl, iso-pentyl, tert-pentyl, 1,2-dimethylpropyl, neopentyl, and 1-ethylpropyl), hexyl groups, and the like. In various embodiments, an alkyl group can have 1 to 40 carbon atoms (i.e., C1-40 alkyl group), for example, 1-30 carbon atoms (i.e., C1-30 alkyl group). In certain embodiments, an alkyl group can have 1 to 6 carbon atoms, and can be referred to as a “lower alkyl group.” Examples of lower alkyl groups include methyl, ethyl, propyl (e.g., n-propyl and isopropyl), and butyl groups (e.g., n-butyl, isobutyl, sec-butyl, tert-butyl). In certain embodiments, alkyl groups can be optionally substituted as described herein. An alkyl group is generally not substituted with another alkyl group, an alkenyl group, or an alkynyl group.

As used herein, “alkenyl” refers to a straight-chain or branched alkyl group having one or more carbon-carbon double bonds. Examples of alkenyl groups include ethenyl, propenyl, butenyl, pentenyl, hexenyl, butadienyl, pentadienyl, hexadienyl groups, and the like. The one or more carbon-carbon double bonds can be internal (such as in 2-butene) or terminal (such as in 1-butene). In various embodiments, an alkenyl group can have 2 to 40 carbon atoms (i.e., C2-40 alkenyl group), for example, 2 to 20 carbon atoms (i.e., C2-20 alkenyl group). In certain embodiments, alkenyl groups can be substituted as described herein. An alkenyl group is generally not substituted with another alkenyl group, an alkyl group, or an alkynyl group.

As used herein, “cycloalkyl” by itself or as part of another substituent means, unless otherwise stated, a monocyclic hydrocarbon having between 3-12 carbon atoms in the ring system and includes hydrogen, straight chain, branched chain, and/or cyclic substituents. Exemplary cycloalkyls include cyclopropyl, cyclobutyl, cyclopentyl, cyclohexyl, cycloheptyl, and the like.

As used herein, “heteroatom” refers to an atom of any element other than carbon or hydrogen and includes, for example, nitrogen, oxygen, silicon, sulfur, phosphorus, and selenium.

The term “heterocycloalkyl” as used herein includes reference to a saturated heterocyclic moiety having 3, 4, 5, 6 or 7 ring carbon atoms and 1, 2, 3, 4 or 5 ring heteroatoms selected from nitrogen, oxygen, phosphorus and sulfur. The group may be a polycyclic ring system but more often is monocyclic. This term includes reference to groups such as azetidinyl, pyrrolidinyl, tetrahydrofuranyl, piperidinyl, oxiranyl, pyrazolidinyl, imidazolyl, indolizidinyl, piperazinyl, thiazolidinyl, morpholinyl, thiomorpholinyl, quinolizidinyl and the like.

As used herein, “aryl” refers to an aromatic monocyclic hydrocarbon ring system or a polycyclic ring system in which two or more aromatic hydrocarbon rings are fused (i.e., having a bond in common with) together or at least one aromatic monocyclic hydrocarbon ring is fused to one or more cycloalkyl and/or heterocycloalkyl rings. An aryl group can have 6 to 24 carbon atoms in its ring system (e.g., C6-24 aryl group), which can include multiple fused rings. In certain embodiments, a polycyclic aryl group can have 8 to 24 carbon atoms. Any suitable ring position of the aryl group can be covalently linked to the defined chemical structure. Examples of aryl groups having only aromatic carbocyclic ring(s) include phenyl, 1-naphthyl (bicyclic), 2-naphthyl (bicyclic), anthracenyl (tricyclic), phenanthrenyl (tricyclic), pentacenyl (pentacyclic), and like groups. Examples of polycyclic ring systems in which at least one aromatic carbocyclic ring is fused to one or more cycloalkyl and/or cycloheteroalkyl rings include, among others, benzo derivatives of cyclopentane (i.e., an indanyl group, which is a 5,6-bicyclic cycloalkyl/aromatic ring system), cyclohexane (i.e., a tetrahydronaphthyl group, which is a 6,6-bicyclic cycloalkyl/aromatic ring system), imidazoline (i.e., a benzimidazolinyl group, which is a 5,6-bicyclic cycloheteroalkyl/aromatic ring system), and pyran (i.e., a chromenyl group, which is a 6,6-bicyclic cycloheteroalkyl/aromatic ring system). Other examples of aryl groups include benzodioxanyl, benzodioxolyl, chromanyl, indolinyl groups, and the like. In certain embodiments, aryl groups can be optionally substituted.

As used herein, “heteroaryl” refers to an aromatic monocyclic ring system containing at least one ring heteroatom selected from oxygen (O), nitrogen (N), sulfur (S), silicon (Si), and selenium (Se) or a polycyclic ring system where at least one of the rings present in the ring system is aromatic and contains at least one ring heteroatom. Polycyclic heteroaryl groups include those having two or more heteroaryl rings fused together, as well as those having at least one monocyclic heteroaryl ring fused to one or more aromatic carbocyclic rings, non-aromatic carbocyclic rings, and/or non-aromatic cycloheteroalkyl rings. A heteroaryl group, as a whole, can have, for example, 5 to 24 ring atoms and contain 1-5 ring heteroatoms (i.e., 5-20 membered heteroaryl group). The heteroaryl group can be attached to the defined chemical structure at any heteroatom or carbon atom that results in a stable structure. Generally, heteroaryl rings do not contain O—O, S—S, or S—O bonds. However, one or more N or S atoms in a heteroaryl group can be oxidized (e.g., pyridine N-oxide thiophene S-oxide, thiophene S,S-dioxide). Examples of heteroaryl groups include, for example, the 5- or 6-membered monocyclic and 5-6 bicyclic ring systems shown below:

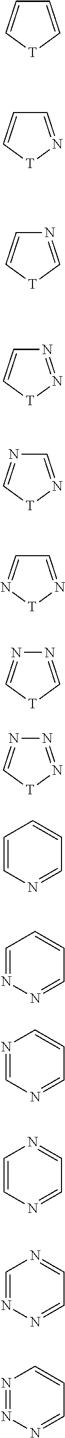

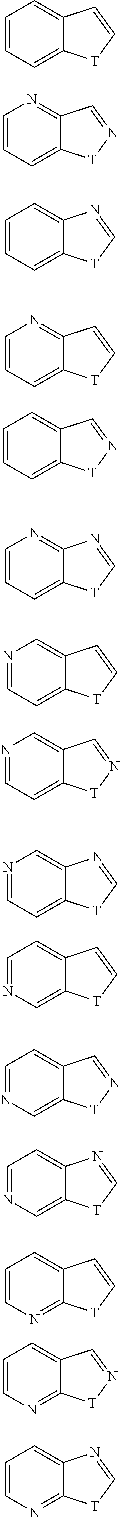

wherein T is O, S, NH, N-alkyl, N-aryl, N-(arylalkyl) (e.g., N-benzyl), SiH₂, SiH(alkyl), Si(alkyl)₂, SiH(arylalkyl), Si(arylalkyl)₂, or Si(alkyl)(arylalkyl). Examples of such heteroaryl rings include pyrrolyl, furyl, thienyl, pyridyl, pyrimidyl, pyridazinyl, pyrazinyl, triazolyl, tetrazolyl, pyrazolyl, imidazolyl, isothiazolyl, thiazolyl, thiadiazolyl, isoxazolyl, oxazolyl, oxadiazolyl, indolyl, isoindolyl, benzofuryl, benzothienyl, quinolyl, 2-methylquinolyl, isoquinolyl, quinoxalyl, quinazolyl, benzotriazolyl, benzimidazolyl, benzothiazolyl, benzisothiazolyl, benzisoxazolyl, benzoxadiazolyl, benzoxazolyl, cinnolinyl, 1H-indazolyl, 2H-indazolyl, indolizinyl, isobenzofuyl, naphthyridinyl, phthalazinyl, pteridinyl, purinyl, oxazolopyridinyl, thiazolopyridinyl, imidazopyridinyl, furopyridinyl, thienopyridinyl, pyridopyrimidinyl, pyridopyrazinyl, pyridopyridazinyl, thienothiazolyl, thienoxazolyl, thienoimidazolyl groups, and the like. Further examples of heteroaryl groups include 4,5,6,7-tetrahydroindolyl, tetrahydroquinolinyl, benzothienopyridinyl, benzofuropyridinyl groups, and the like. In certain embodiments, heteroaryl groups can be substituted as described herein. In certain embodiments, heteroaryl groups can be optionally substituted.

The term “heterocyclic group” refers to a cyclic group containing one or more heteroatoms, including heterocyclcoalkyl, heteroaryl, alkylheteroaryl, and heteroalkylaryl groups. The examples may contain, but not limited to, piperidine, oxane, azepine, morpholine and the like.

The term “aralkyl” refers to an alkyl group substituted with an aryl group.

The term “optionally substituted” refers to a chemical group, such as alkyl, cycloalkyl aryl, and the like, wherein one or more hydrogen may be replaced with a substituent as described herein, for example, halogen, azide, alkyl, aralkyl, alkenyl, alkynyl, cycloalkyl, hydroxyl, alkoxyl, amino, nitro, sulfhydryl, imino, amido, phosphonate, phosphinate, carbonyl, carboxyl, silyl, ether, alkylthio, sulfonyl, sulfonamido, ketone, aldehyde, ester, heterocyclyl, aromatic or heteroaromatic moieties, —CF₃, —CN, or the like

The term “nitro” is art-recognized and refers to —NO₂; the term “halogen” is art-recognized and refers to —F, —Cl, —Br or —I; the term “sulfhydryl” is art-recognized and refers to —SH; the term “hydroxyl” means-OH; and the term “sulfonyl” and “sulfone” is art-recognized and refers to —SO₂—. “Halide” designates the corresponding anion of the halogens.

The symbol “ [symbol] ” or “ [symbol] ” or “ [symbol] ” or “ [symbol] ” in a chemical structure represents a position from where the specified chemical structure is bonded to another chemical structure.

“Enantiomers” are a pair of stereoisomers that are non-superimposable mirror images of each other. A mixture of a pair of enantiomers in equal proportions can be known as a “racemic” mixture. The term “(+/−)” is used to designate a racemic mixture where appropriate. The absolute stereochemistry can be specified according to the Cahn-Ingold-Prelog R-S system. When a compound is an enantiomer, the stereochemistry at each chiral carbon and/or axis of chirality can be specified by either R or S. Resolved compounds whose absolute configuration is unknown can be designated (+) or (−) depending on the direction (dextro- or levorotatory) which they rotate plane polarized light at the wavelength of the sodium D line. Certain compounds described herein can contain one or more asymmetric centers and/or axis of chirality and can thus give rise to enantiomers, diastereomers, and other stereoisomeric forms that can be defined, in terms of absolute stereochemistry at each asymmetric atom or axis of chirality, as (R)— or (S)—. The present compounds and methods are meant to include all such possible isomers, including substantially enantiopure forms and intermediate mixtures. Optically active (R)- and (S)-isomers can be prepared, for example, using chiral synthons or chiral reagents, or resolved using conventional techniques.

The “enantiomeric excess” or “% enantiomeric excess” of a composition can be calculated using the equation shown below. In the example shown below, a composition contains 90% of one enantiomer, e.g., an S enantiomer, and 10% of the other enantiomer, e.g., an R enantiomer. ee=(90−10)/100=80%.

Thus, a composition containing 90% of one enantiomer and 10% of the other enantiomer is said to have an enantiomeric excess of 80%. Some compositions described herein contain an enantiomeric excess of at least about 1%, about 5%, about 10%, about 20%, about 30%, about 40%, about 50%, about 75%, about 90%, about 95%, about 99%, or greater of the S enantiomer. In other words, the compositions contain an enantiomeric excess of the S enantiomer over the R enantiomer. In other embodiments, some compositions described herein contain an enantiomeric excess of at least about 1%, about 5%, about 10%, about 20%, about 30%, about 40%, about 50%, about 75%, about 90%, about 95%, about 99%, or greater of the R enantiomer. In other words, the compositions contain an enantiomeric excess of the R enantiomer over the S enantiomer.

For instance, an enantiomer can, in some embodiments, be provided substantially free of the corresponding enantiomer, and can also be referred to as “optically enriched,” “enantiomerically enriched,” “enantiomerically pure”, “substantially enantiopure” and “non-racemic,” as used interchangeably herein. These terms refer to compositions in which the amount of one enantiomer is greater than the amount of that one enantiomer in a control mixture of the racemic composition (e.g., greater than 1:1 by weight). For example, an enantiomerically enriched preparation of the S enantiomer, means a preparation of the compound having greater than about 50% by weight of the S enantiomer relative to the total weight of the preparation (e.g., total weight of S and R isomers), such as at least about 75% by weight, further such as at least about 80% by weight. In some embodiments, the enrichment can be much greater than about 80% by weight, providing a “substantially enantiomerically enriched,” “substantially enantiomerically pure” or a “substantially non-racemic” preparation, which refers to preparations of compositions which have at least about 70% by weight of one enantiomer relative to the total weight of the preparation, such as at as at least about 75% by weight, such as at as at least about 80% by weight, such as at as at least about 85% by weight, such as at least about 90% by weight, and such as at least about 95% by weight. In certain embodiments, the compound provided herein is made up of at least about 90% by weight of one enantiomer. In other embodiments, the compound is made up of at least about 95%, about 98%, or about 99% by weight of one enantiomer.

In certain embodiments, the compound is a racemic mixture of (S)- and (R) isomers. In other embodiments, provided herein is a mixture of compounds wherein individual compounds of the mixture exist predominately in an (S)- or (R)-isomeric configuration. For example, in certain embodiments, the compound mixture has an (S)-enantiomeric excess of greater than about 10%, greater than about 20%, greater than about 30%, greater than about 40%, greater than about 50%, greater than about 55%, greater than about 60%, greater than about 65%, greater than about 70%, greater than about 75%, greater than about 80%, greater than about 85%, greater than about 90%, greater than about 95%, greater than about 96%, greater than about 97%, greater than about 98%, or greater than about 99%. In certain embodiments, the compound mixture has an (S)-enantiomeric excess of about 55%, about 60%, about 65%, about 70%, about 75%, about 80%, about 85%, about 90%, about 95%, about 96%, about 97%, about 98%, about 99%, or about 99.5%, or more. In certain embodiments, the compound mixture has an (S)-enantiomeric excess of about 55% to about 99.5%, about 60% to about 99.5%, about 65% to about 99.5%, about 70% to about 99.5%, about 75% to about 99.5%, about 80% to about 99.5%, about 85% to about 99.5%, about 90% to about 99.5%, about 95% to about 99.5%, about 96% to about 99.5%, about 97% to about 99.5%, about 98% to about 99.5%, or about 99% to about 99.5%, or more than about 99.5%.

In other embodiments, the compound mixture has an (R)-enantiomeric excess of greater than about 10%, greater than about 20%, greater than about 30%, greater than about 40%, greater than about 50%, greater than about 55%, greater than about 60%, greater than about 65%, greater than about 70%, greater than about 75%, greater than about 80%, greater than about 85%, greater than about 90%, greater than about 95%, greater than about 96%, greater than about 97%, greater than about 98%, or greater than about 99%. In certain embodiments, the compound mixture has an (R)-enantiomeric excess of about 55%, about 60%, about 65%, about 70%, about 75%, about 80%, about 85%, about 90%, about 95%, about 96%, about 97%, about 98%, about 99%, or about 99.5%, or more. In certain embodiments, the compound mixture has an (R)-enantiomeric excess of about 55% to about 99.5%, about 60% to about 99.5%, about 65% to about 99.5%, about 70% to about 99.5%, about 75% to about 99.5%, about 80% to about 99.5%, about 85% to about 99.5%, about 90% to about 99.5%, about 95% to about 99.5%, about 96% to about 99.5%, about 97% to about 99.5%, about 98% to about 99.5%, or about 99% to about 99.5%, or more than about 99.5%.

Provided herein is a compound of Formula 1, 2, 3, or 4:

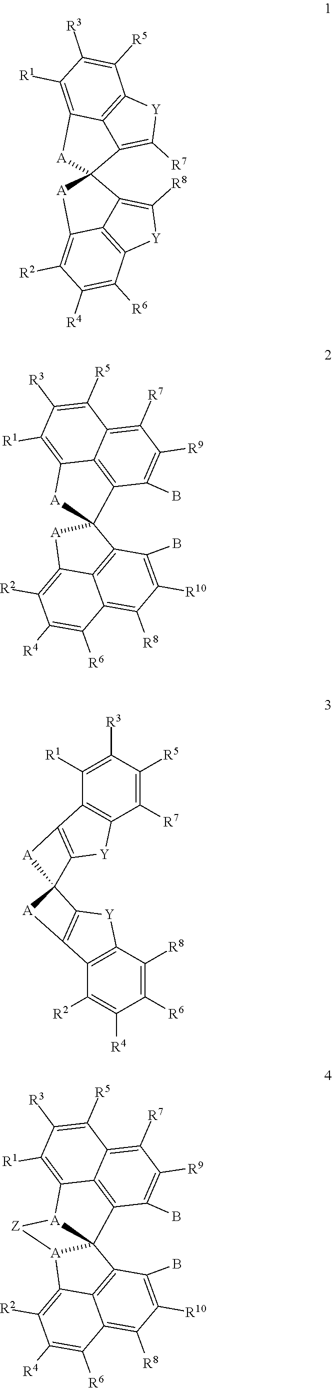

or a conjugate salt thereof, wherein:
A is —(CR₂)ₙ—, wherein n for each instance is independently a whole number selected from 1-5 and R for each instance is independently hydrogen, alkyl, cycloalkyl, aryl, aralkyl, or a covalent bond to Z;
each B is independently —CH₂OR²⁰, —CH₂NR²⁰₂, —OR²⁰, —NR²⁰₂, —PR²⁰₂, —OP(OR²⁰)₂, —NHPR²⁰₂, —NHP(OR²⁰)₂, —NH(C═S)NHR²⁰, or a moiety represented by:

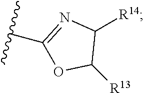

or
two instances of B taken together form a moiety having a structure selected from the group consisting of:

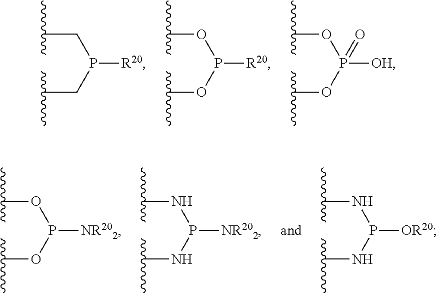

each Y is independently selected from the group consisting of: —O—, —S—, —(NR²⁰)—, and —(NPR²⁰₂)—;
Z is —(CR₂)ₙ—, wherein n for each instance is independently a whole number selected from 1-5 and R for each instance is independently hydrogen, alkyl, cycloalkyl, aryl, or aralkyl;
each of R¹, R², R³, R⁴, R⁵, R⁶, R⁷, R⁸, R⁹, and R¹⁰ is independently selected from the group consisting of hydrogen, alkyl, cycloalkyl, heterocycloalkyl, aralkyl, aryl, heteroaryl, halide, trialkylsilane, and triarylsilane; or
each of R⁷ and R⁸ is independently —CH₂OR²⁰, —CH₂NR²⁰₂, —OR²⁰, —NR²⁰₂, —PR²⁰₂, —OP(OR²⁰)₂, —NHPR²⁰₂, —NHP(OR²⁰)₂, or a moiety represented by:

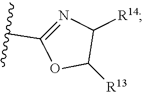

or
R⁷ and R⁸ is taken together form a moiety having a structure selected from the group consisting of:

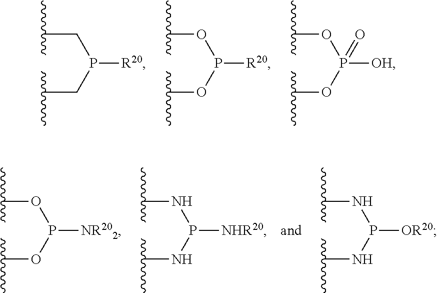

each of R¹³ and R¹⁴ is independently, hydrogen, alkyl, cycloalkyl, aryl, or aralkyl; and
R²⁰ for each instance is independently hydrogen, alkyl, cycloalkyl, heterocycloalkyl, aryl, aralkyl, heteroaryl, or —CHPhCH₂PPh₂; or two instances of R²⁰ taken together form 5-7 membered heterocyclic group.

In the interest of clarity, certain compounds described herein may be shown as a single enantiomer. However, the present disclosure contemplates all isomeric forms of the compound described herein, including all stereoisomers.

The compound of Formula 1, 2, 3, or 4 can be substantially enantiomerically pure. In certain embodiments, the compound of Formula 1, 2, 3, and 4 has an enantiomeric excess of either the R-enantiomer or the S-enantiomer of 50-100%, 50-99.9%, 60-99.9%, 70-99.9%, 80-99.9%, 90-99.9%, 95-99.9%, 97-99.9%, or 99-99.9%.

Conjugate salts include quaternary ammonium salts of the compounds described herein, e.g., from organic or inorganic acids. For example, such salts include those derived from inorganic acids such as hydrochloride, hydrobromic, sulfuric, sulfamic, phosphoric, nitric, and the like; and the salts prepared from organic acids.

In other cases, the compounds described herein may contain one or more acidic functional groups and, thus, are capable of forming conjugate salts with bases. The term conjugate salts in these instances refer to inorganic and organic base addition salts of compounds described herein. Representative alkali or alkaline earth salts include the lithium, sodium, potassium, calcium, magnesium, and the like.

In certain embodiments, R for each instance is independently selected from the group consisting of C₁-C₆ alkyl, C₁-C₅alkyl, C₁-C₄ alkyl, C₁-C₃ alkyl, C₁-C₂ alkyl, C₃-C₇ cycloalkyl, C₃-C₆ cycloalkyl, C₃-C₈ cycloalkyl, C₃-C₄ cycloalkyl, C₆-C₁₈ aryl, C₆-C₁₄ aryl, or C₆-C₁₀ aryl. In certain embodiments, R for each instance is independently selected from the group consisting of hydrogen, methyl, ethyl, propyl, isopropyl, sec-butyl, tert-butyl, cyclohexyl, and phenyl.

In certain embodiments, n is 1-5, 1-4, 1-3, 1-2, 2-5, 3-5, or 4-5. In certain embodiments, n is 1, 2, 3, 4, or 5. In the examples below n is 1.

In certain embodiments, A is —(CR₂)ₙ—, wherein one instance of R is alkyl, cycloalkyl, aryl, or aralkyl; and the each of the remaining R is hydrogen. In instances in which wherein one instance of R is alkyl, cycloalkyl, aryl, or aralkyl; and the remaining R are hydrogen, the carbon bearing the R, which is alkyl, cycloalkyl, aryl, or aralkyl is substantially enantiomerically pure. In certain embodiments, A is —(CH₂)ₙ—, —(CHR)(CH₂)ₙ₋₁—, or —(CH₂)ₙ₋₁ (CHR)—, wherein R is as defined in any embodiment described herein. In certain embodiments, A is —(CH₂)ₙ—, —(CHR)(CH₂)ₙ₋₁—, or —(CH₂)ₙ₋₁ (CHR)—, wherein R is phenyl.

Each B can independently be —CH₂OR²⁰, —CH₂NR²⁰₂, —OR²⁰, —NR²⁰₂, —PR²⁰₂, —OP(OR²⁰)₂, —NHPR²⁰₂, —NHP(OR²⁰)₂, —NH(C═S)NHR²⁰, or a moiety represented by:

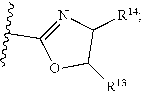

or
two instances of B taken together form a moiety having a structure selected from the group consisting of:

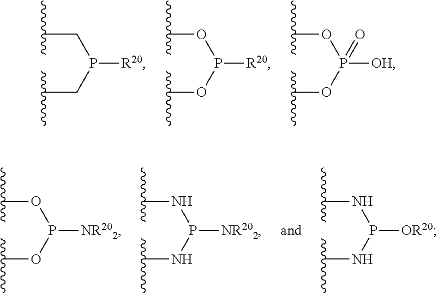

In certain embodiments, the compound has Formula 2 and each B is independently —CH₂OR²⁰, —CH₂NR²⁰₂, —OR²⁰, —NR²⁰₂, —PR²⁰₂, —NHPR²⁰₂, —OP(OR²⁰)₂, —NH(C═S)NHR²⁰, or a moiety represented by:

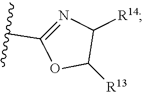

or
two instances of B taken together form a moiety represented by:

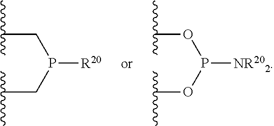

In certain embodiments, the compound has Formula 2 and each B is independently —NHPR²⁰₂, —OP(OR²⁰)₂, —NH(C—S)NHCHPhCH₂PPh₂, —OR²⁰, or a moiety represented by:

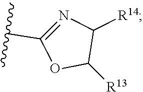

or
two instances of B taken together form a moiety represented by:

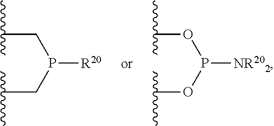

wherein R²⁰ for each instance is independently alkyl, cycloalkyl, or aryl.

In certain embodiments, the compound has Formula 1 or 3 and each Y is —(NPR²⁰₂)—.

In instances in which B is a moiety represented by:

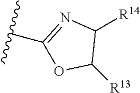

and one or more of R¹³ and R¹⁴ is not hydrogen (i.e., is alkyl, cycloalkyl, aryl, or aralkyl), the carbon bearing the R¹³ and/or R¹⁴ non-hydrogen moiety can be substantially enantiomerically pure. In certain embodiments, R¹³ is hydrogen and R¹⁴ is isopropyl, tert-butyl, phenyl, or benzyl. In certain embodiments, R¹³ is hydrogen and R¹⁴ is benzyl.

Each Y can independently be selected from the group consisting of: —O—, —S—, —(NR²⁰)—, and —(NPR²⁰₂)—. In certain embodiments, each Y is —(NPR²⁰₂)—.

In certain embodiments, Z is —(CH₂)ₙ—, wherein n for each instance is independently a whole number selected from 2 or 3.

Each of R¹, R², R³, R⁴, R⁵, R⁶, R⁷, R⁸, R⁹, and R¹⁰ can independently be selected from the group consisting of hydrogen, alkyl, cycloalkyl, aryl, and halide. In certain embodiments, each of R¹, R², R³, R⁴, R⁵, R⁶, R⁷, R⁸, R⁹, and R¹⁰ is independently selected from the group consisting of hydrogen, C₁-C₆ alkyl, C₁-C₅alkyl, C₁-C₄ alkyl, C₁-C₃ alkyl, C₁-C₂ alkyl, C₃-C₇ cycloalkyl, C₃-C₆ cycloalkyl, C₃-C₅ cycloalkyl, C₃-C₄ cycloalkyl, C₆-C₁₈ aryl, C₆-C₁₄ aryl, C₆-C₁₀ aryl, chloride, bromide, and iodide. In certain embodiments, each of R¹, R², R³, R⁴, R⁵, R⁶, R⁷, R⁸, R⁹, and R¹⁰ is hydrogen.

In certain embodiments, each of R⁷ and R⁸ is —PR²⁰₂.

In certain embodiments, each of R¹³ and R¹⁴ is independently, hydrogen, C₁-C₆ alkyl, C₁-C₅alkyl, C₁-C₄ alkyl, C₁-C₃ alkyl, C₁-C₂ alkyl, C₃-C₇ cycloalkyl, C₃-C₆ cycloalkyl, C₃-C₅ cycloalkyl, C₃-C₄ cycloalkyl, C₆-C₁₈ aryl, C₆-C₁₄ aryl, or C₆-C₁₀ aryl. In certain embodiments, each of R¹³ and R¹⁴ is independently, hydrogen, methyl, isopropyl, tert-butyl, cyclohexyl, or phenyl.

R²⁰ can independently be selected from the group consisting of hydrogen, C₁-C₆ alkyl, C₁-C₅alkyl, C₁-C₄ alkyl, C₁-C₃ alkyl, C₁-C₂ alkyl, C₃-C₇ cycloalkyl, C₃-C₆ cycloalkyl, C₃-C₅ cycloalkyl, C₃-C₄ cycloalkyl, C₆-C₁₈ aryl, C₆-C₁₄ aryl, C₆-C₁₀ aryl, or —CHPhCH₂PPh₂. In instances in which R20 is —CHPhCH₂PPh₂, the stereogenic carbon labeled with * in the structure: —C*HPhCH₂PPh₂ can be substantially enantiomerically pure.

In certain embodiments, the compound described herein, e.g., chiral spirocyclic diamine, chiral spirocyclic amino naphthol, chiral spirocyclic bis(indole), chiral spirocyclic diaryl diol, chiral spirocyclic diaryl diamine, chiral spirocyclic amino naphthol, chiral spirocyclic diaryl diindole, chiral spirocyclic phospholane, can be represented by the following formula:

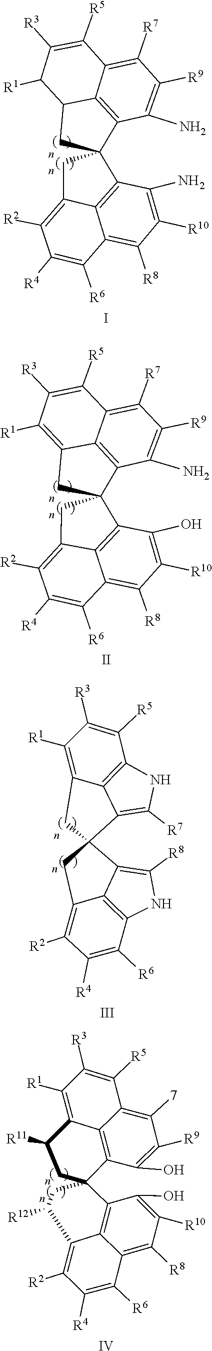

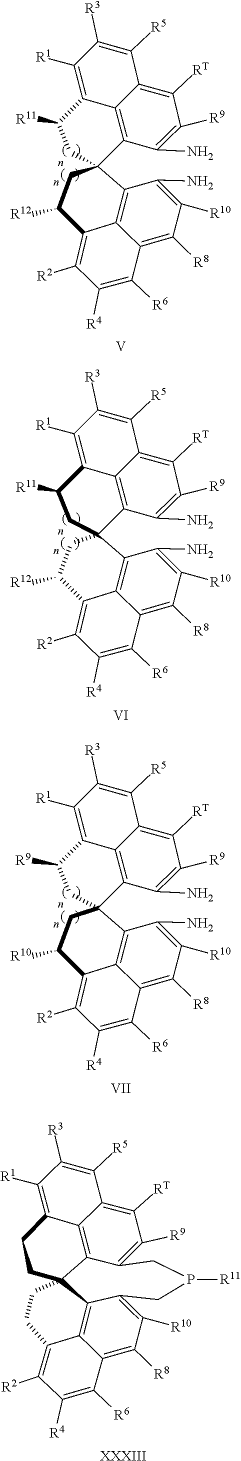

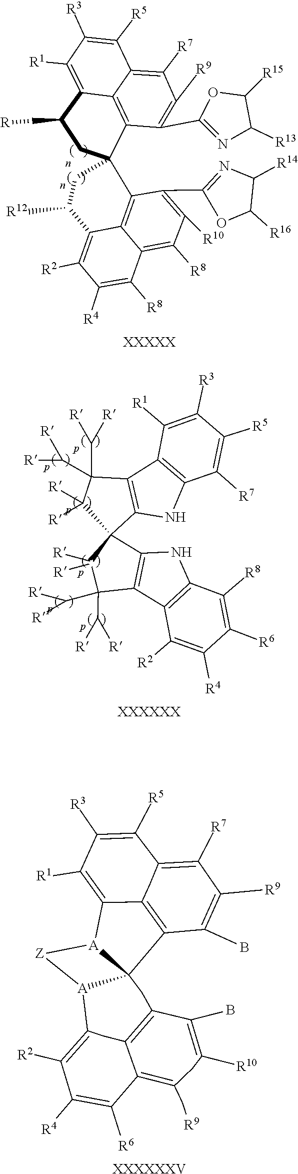

wherein: R′, R¹, R², R³, R⁴, R⁵, R⁶, R⁷, R⁸, R⁹, R¹⁰, R¹¹, R¹², R¹³, R¹⁴, R¹⁵, and R¹⁶ independently represent C₁₋₁₀₀ alkyl groups, alkoxyl groups, aryl groups, halogen or hydrogen; p is a whole number selected from 1-5; and each of n, A, B, and Z is independently as defined in any embodiment described herein. The number of ‘n’ is 1 to 5.

In certain embodiments, the compound is selected from the group consisting of:

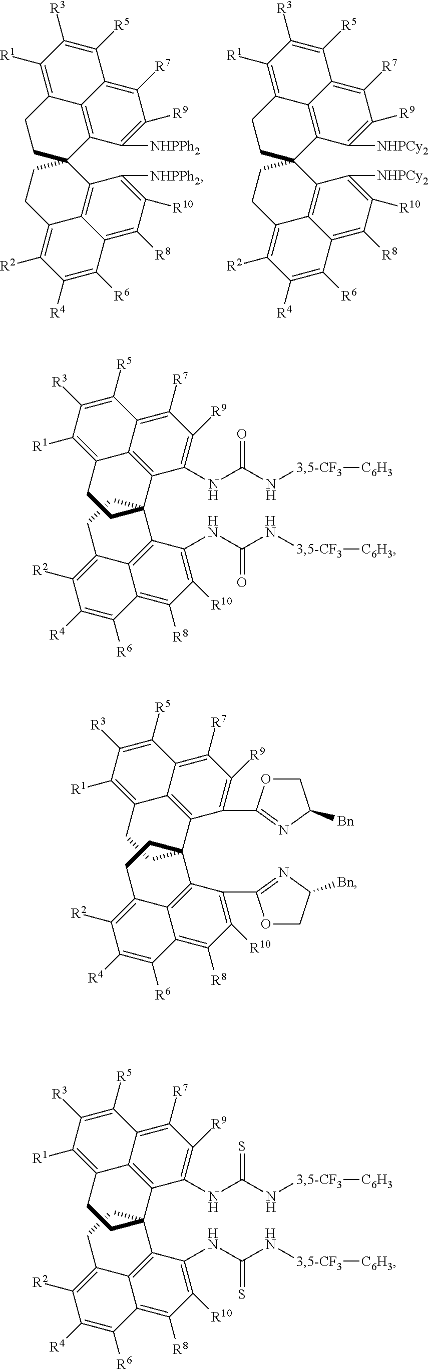

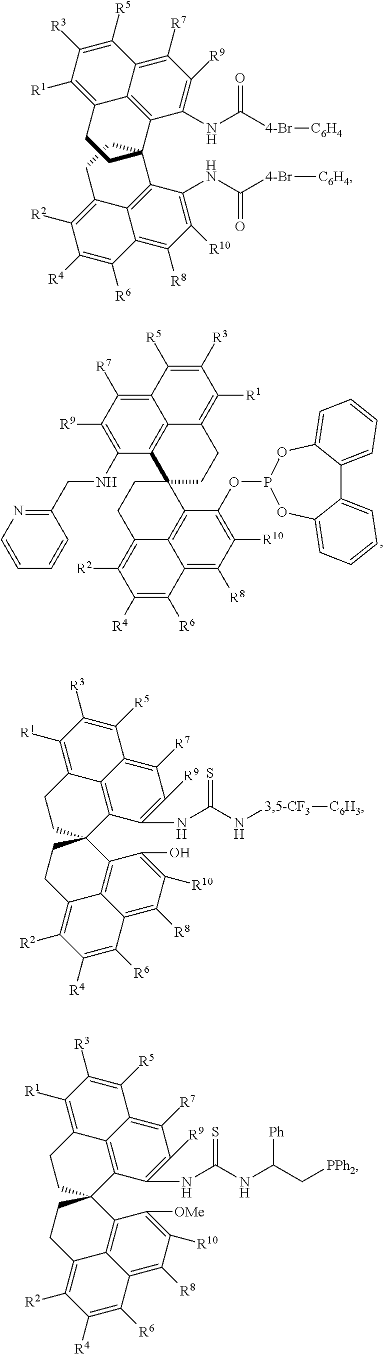

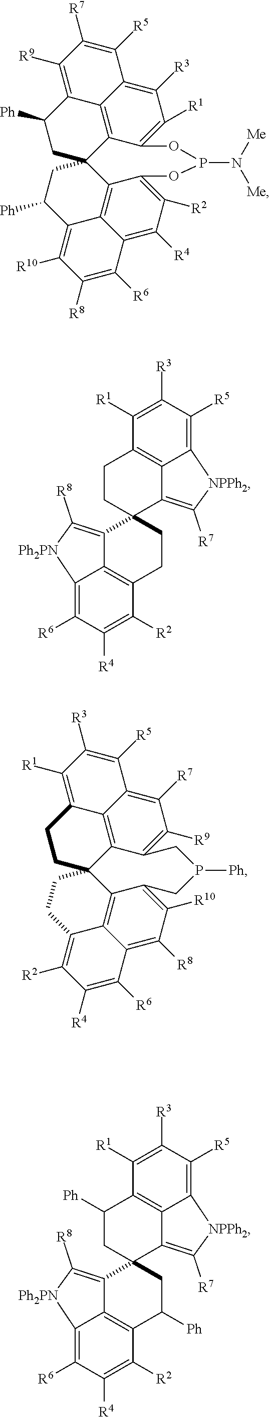

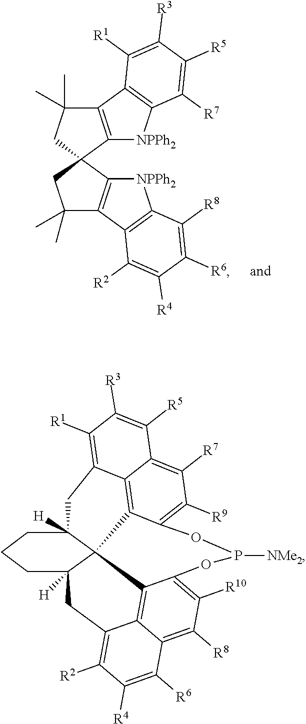

wherein each of R¹, R², R³, R⁴, R⁵, R⁶, R⁷, R⁸, R⁹, and R¹⁰ is independently as defined in any embodiment described herein.

In certain embodiments, the compound is selected from the group consisting of:

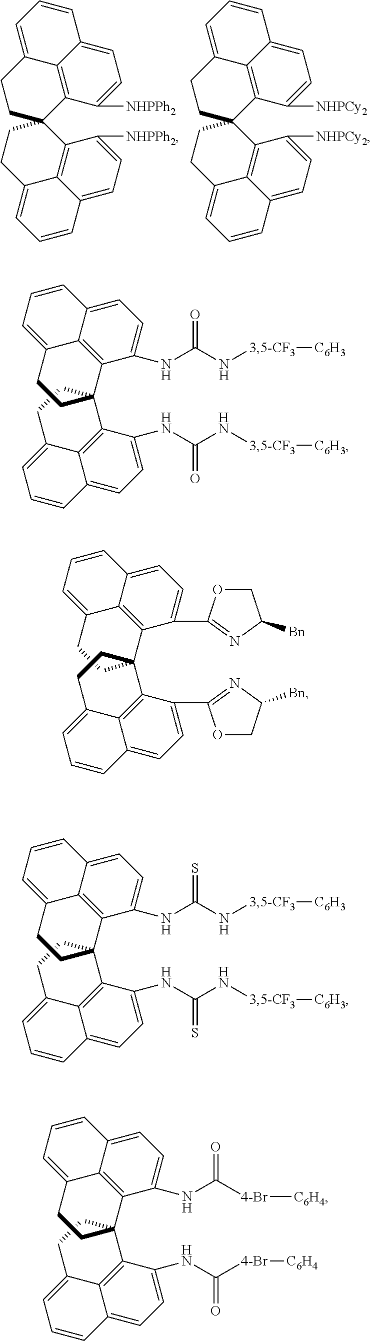

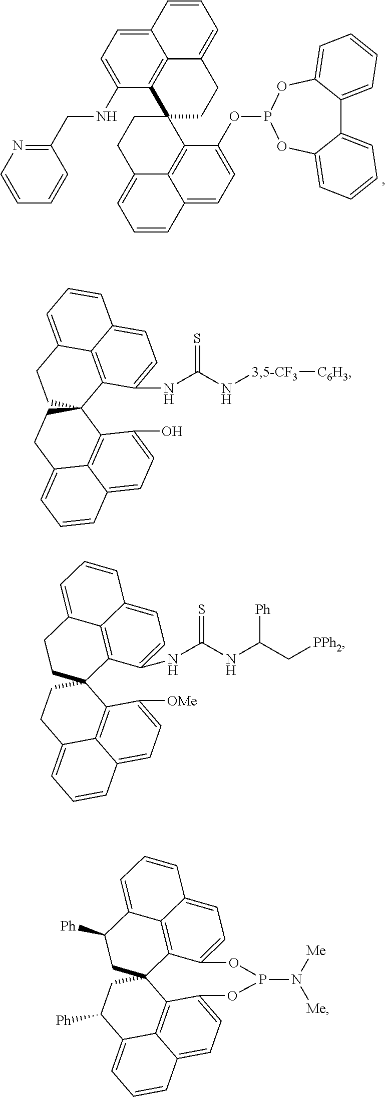

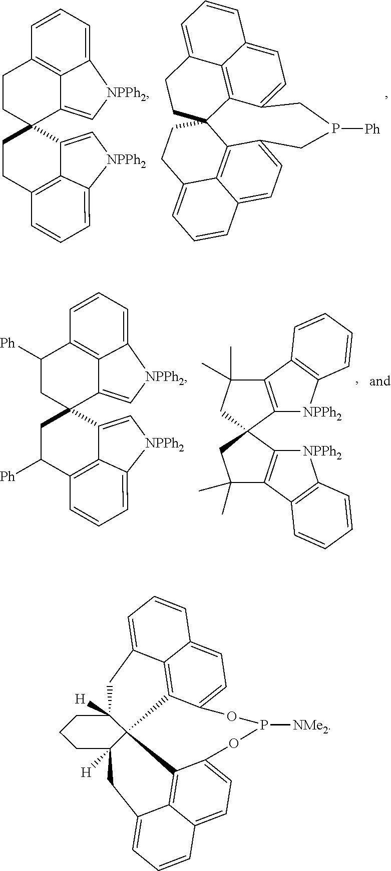

The present disclosure also provides a catalyst or pre-catalyst precursor comprising the compound described herein and a metal.

The metal is not particularly limited, and the present disclosure contemplates all metals useful in catalytic reactions. Exemplary metals include, but are not limited to, copper, nickel, iron, gold, cobalt, manganese, molybdenum, titanium, silver, palladium, platinum, iridium, ruthenium, or rhodium.

Compounds described herein bearing nucleophilic moieties, such as thiourea, phosphine, amines, and the like; or Brønsted acidic moieties, such as phosphoric acids, can be used as organocatalysts.

The present disclosure also provides a method of use of a catalyst or a pre-catalyst comprising the compound described herein and a metal; or a compound described herein as an organocatalyst, the method comprising contacting the catalyst, pre-catalyst, or organocatalyst with a substrate and optionally one or more reagents under conditions to induce chemical reaction thereby forming a reaction product. The compounds described herein are useful in the preparation of enantioselective rhodium hydrogenation catalysts, enantioselective iridium hydrogenation catalysts, enantioselective palladium allylic substitution catalysts, and enantioselective copper allylic oxidation catalysts; as well as organocatlysts for enantioselective Michael addition and enantioselective [3+2] cycloaddition.

The compounds described herein can be readily prepared by a method comprising: contacting a compound of Formula 5 or 6:

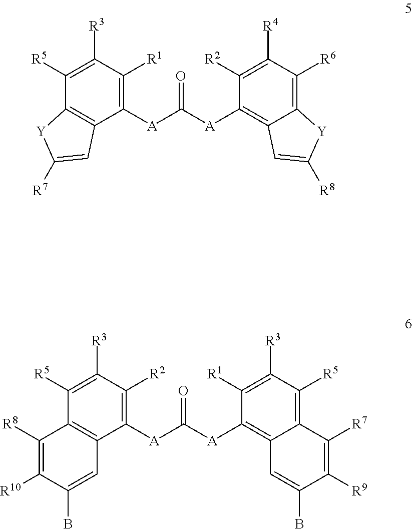

wherein
A is —(CR₂)ₙ—, wherein n is a whole number selected from 1-5 and R for each instance is independently hydrogen, alkyl, cycloalkyl, aryl, or aralkyl;
each B is independently —OR²⁰ or —NR²⁰₂; and
each of R¹, R², R³, R⁴, R⁵, R⁶, R⁷, R⁸, R⁹, and R¹⁰ is independently selected from the group consisting of hydrogen, alkyl, cycloalkyl, heterocycloalkyl, aralkyl, aryl, heteroaryl, halide, trialkylsilane, and triarylsilane; and
R²⁰ for each instance is independently hydrogen, alkyl, cycloalkyl, heterocycloalkyl, aryl, aralkyl, heteroaryl, or —CHPhCH₂PPh₂; or two instances of R²⁰ taken together form 5-7 membered heterocyclic group;
with a phosphoric acid selected from the group consisting of:

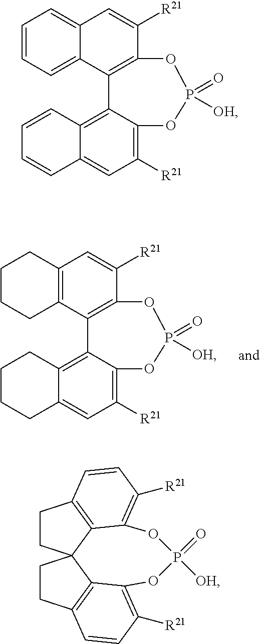

R²¹ is aryl or triarylsilyl thereby forming the compound or synthon thereof.

In certain embodiments, the phosphoric acid is substantially enantiomerically pure.

In certain embodiments, the phosphoric acid is:

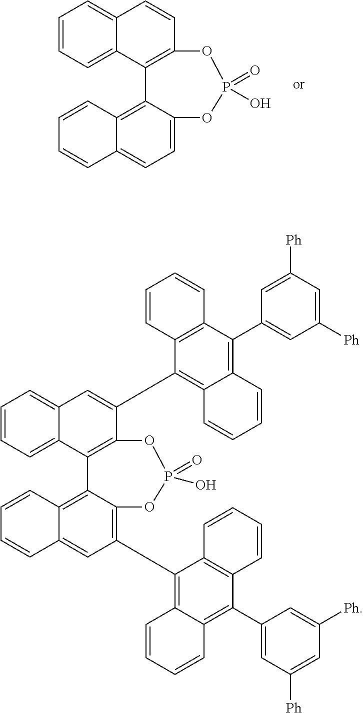 [SMILES: O=P1(O)Oc2ccc3ccccc3c2-c2c(ccc3ccccc23)O1 ; O=P1(O)Oc2c(-c3c4ccccc4c(-c4cc(-c5ccccc5)cc(-c5ccccc5)c4)c4ccccc34)cc3ccccc3c2-c2c(c(-c3c4ccccc4c(-c4cc(-c5ccccc5)cc(-c5ccccc5)c4)c4ccccc34)cc3ccccc23)O1]

The compound of Formula 5 or 6 and the phosphoric acid can be contacted in any solvent in which they are at least partially soluble. The solvent can be an aromatic solvent, a haloaromatic solvent, a haloalkane, or a combination thereof. In certain embodiments, the solvent is selected from the group consisting of PhMe, PhCl, PhCF₃, PhF, CCl₄, CH₂Cl₂, CHCl₃, and combinations thereof.

The phosphoric acid can be present at 0.01-100 mol %, 0.01-75 mol %, 0.01-50 mol %, 0.01-25 mol %, 0.01-20 mol %, 0.01-15 mol %, 0.01-10 mol %, 1-10 mol %, 1-9 mol %, 1-8 mol %, 1-7 mol %, 1-6 mol %, 1-5 mol %, 1-4 mol %, 1-3 mol %, 1-2 mol %, 5-15 mol %, 6-14 mol %, 7-13 mol %, 8-12 mol %, 9-11 mol %, 15-25 mol %, 16-24 mol %, 17-23 mol %, 18-22 mol %, or 19-21 mol % relative to the compound of Formula 5 or 6.

The compound of Formula 1, 2, 3, or 4 and the phosphoric acid can be contacted at a temperature of 0-70° C., 0-60° C., 0-50° C., 0-40° C., 0-30° C., 0-20° C., 0-10° C., 10-70° C., 20-70° C., 30-70° C., 40-70° C., 50-70° C., 60-70° C., 10-60° C., 20-50° C., or 30-40° C.

A wide range of chiral spirocyclic diamine I, chiral spirocyclic amino naphthol II, chiral spirocyclic bis(indole) III, chiral biaryl spirocyclic diol IV, chiral biaryl spirocyclic diamine V, chiral biaryl spirocyclic amino naphthol VI, chiral biaryl spirocyclic bis(indole) VII, and chiral cycloalkane-fused spirocyclic diol XXXXXXV were synthesized from ketone VIII, IX, X, XI, XII, XIII, XIV, and XXXXXXVI, respectively using the methods described herein. Chiral spirocyclic phospholane XXXXIII was synthesized from SPHENOL. Chiral spirocyclic bis(indole) XXXXXX was synthesized from indole XXXXXX-1 and ketone XXXXXX-2.

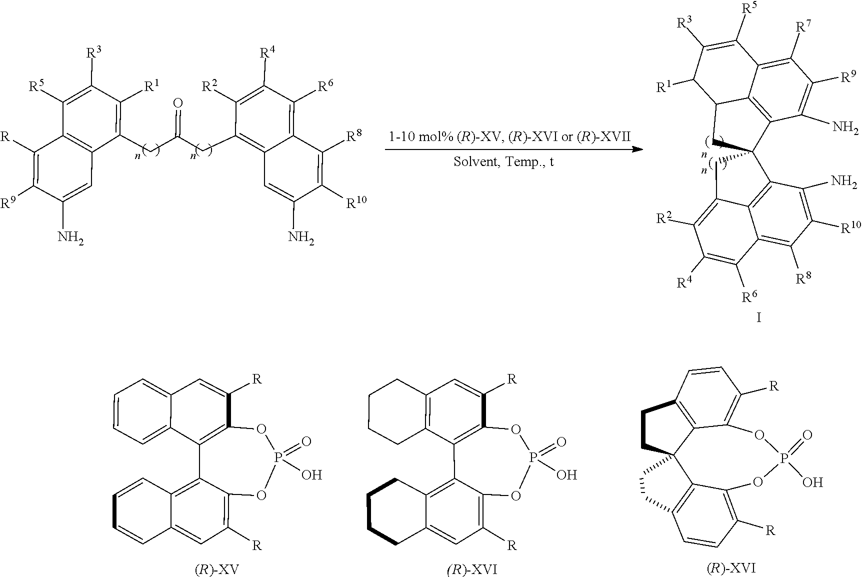

The catalyst is a chiral phosphoric acid with BINOL (XV), 8H-BINOL (XVI), or SPINOL (XVII) backbone, in which the 3,3′-substitution group R represents polycyclic aromatic hydrocarbons such as substituted benzene, naphthalene, fluorene, pyrene, anthracene phenanthrene or triarylsilyl.

The catalyst here can be some other acids containing different chiral backbones.

The solvent can be chlorobenzene, PhCF₃, PhF, CCl₄, DCM, CHCl₃, PhMe, and other organic solvents.

The temperature can range from 0° C. to 70° C. In certain embodiments, the temperature is 70° C.

The invention provides a process for the synthesis of an array of chiral spiro diamines like I. These compounds can also be transformed to other useful compounds such as chiral ligands and chiral catalysts by simple chemical steps. The enantioselectivity can generally remain without erosion.

This process represents the first catalytic enantioselective synthetic preparation of chiral SPHENAM.

R¹, R², R³, R⁴, R⁵, R⁶, R⁷, R⁸, R⁹, and R¹⁰ independently represent C₁₋₁₀₀ alkyl groups, alkoxyl groups, aryl groups, halogen or hydrogen. The number of ‘n’ was chosen from 1 to 5.

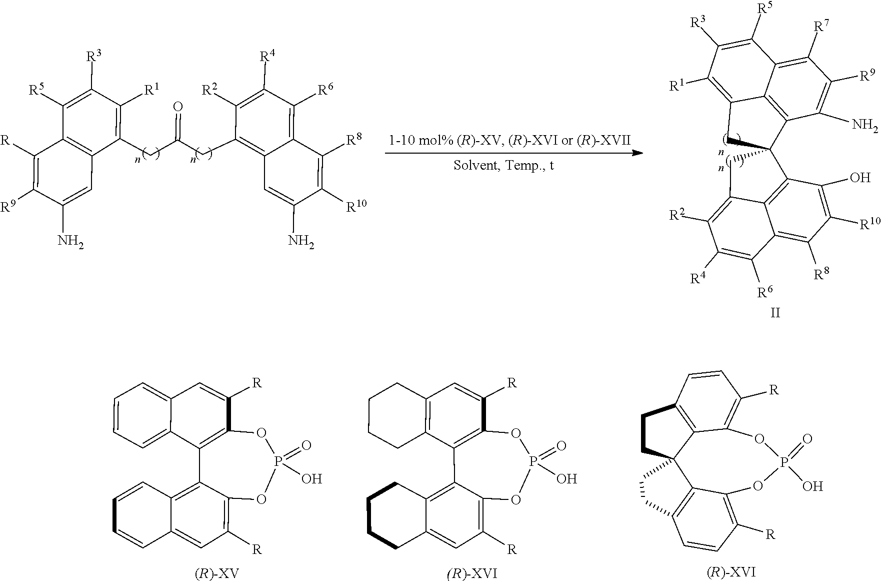

The catalyst is a chiral phosphoric acid with BINOL (XV), 8H-BINOL (XVI), or SPINOL (XVII) backbone, in which the 3,3′-substitution group R represents polycyclic aromatic hydrocarbons such as substituted benzene, naphthalene, fluorene, pyrene, anthracene phenanthrene or triarylsilyl.

The catalyst here can be some other acids containing different chiral backbones.

The solvent can be chlorobenzene, PhCF₃, PhF, CCl₄, DCM, CHCl₃, PhMe, and other organic solvents.

The temperature can range from 0° C. to 70° C. In certain embodiments, the temperature is 70° C.

The invention provides a process for the synthesis of an array of chiral spiro amino naphthols like II. These compounds can also be transformed to other useful compounds such as chiral ligands and chiral catalysts by simple chemical steps. The enantioselectivity can generally remain without erosion.

This process represents the first catalytic enantioselective synthetic preparation of chiral NOSPHEN.

R¹, R², R³, R⁴, R⁵, R⁶, R⁷, and R⁸ independently represent C₁₋₁₀₀ alkyl groups, alkoxyl groups, aryl groups, halogen or hydrogen. The number of ‘n’ was chosen from 1 to 5.

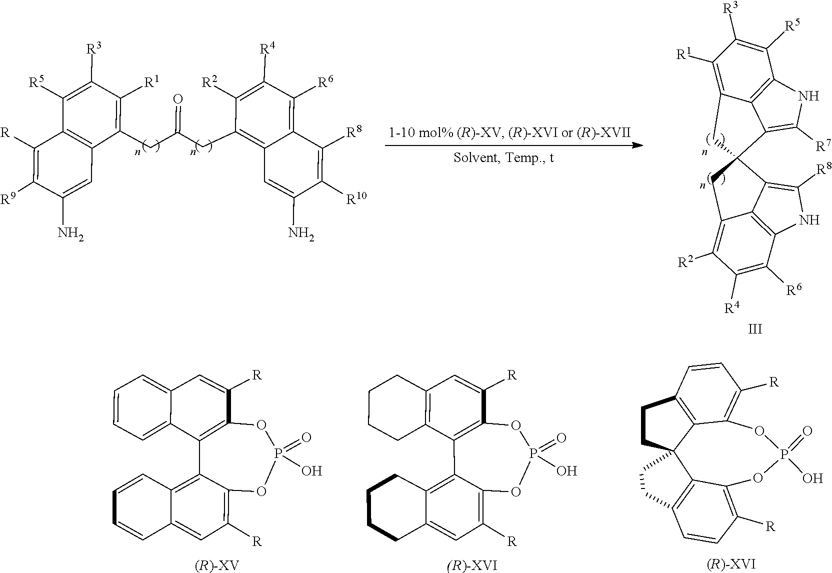

The catalyst is a chiral phosphoric acid with BINOL (XV), 8H-BINOL (XVI), or SPINOL (XVII) backbone, in which the 3,3′-substitution group R represents polycyclic aromatic hydrocarbons such as substituted benzene, naphthalene, fluorene, pyrene, anthracene phenanthrene or triarylsilyl.

The catalyst here can be some other acids containing different chiral backbones.

The solvent can be chlorobenzene, PhCF₃, PhF, CCl₄, DCM, CHCl₃, PhMe, and other organic solvents.

The temperature can range from 0° C. to 70° C. In certain embodiments, the temperature is 60° C.

The invention provides a process for the synthesis of an array of chiral spiro bis(indole) s like III. These compounds can also be transformed to other useful compounds such as chiral ligands and chiral catalysts by simple chemical steps. The enantioselectivity can generally remain without erosion.

This process represents the first catalytic enantioselective synthetic preparation of chiral spiro bis(indole) III.

R¹, R², R³, R⁴, R⁵, R⁶, R⁷, R⁸, R⁹, R¹⁰, R¹¹, and R¹² independently represent C₁₋₁₀₀ alkyl groups, alkoxyl groups, aryl groups, halogen or hydrogen. The number of ‘n’ was chosen from 1 to 5.

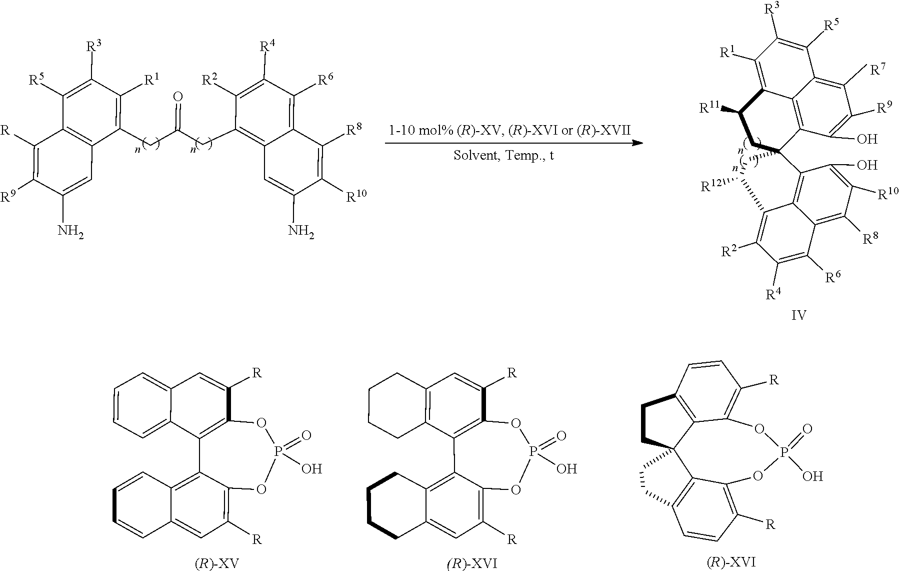

The catalyst is a chiral phosphoric acid with racemic BINOL (XV), racemic 8H-BINOL (XVI), or racemic SPINOL (XVII) backbone, in which the 3,3′-substitution group R represents hydrogen or polycyclic aromatic hydrocarbons such as substituted benzene, naphthalene, fluorene, pyrene, anthracene phenanthrene or triarylsilyl.

The catalyst here can be some other acids containing different chiral backbones.

The solvent can be chlorobenzene, PhCF₃, PhF, CCl₄, DCM, CHCl₃, PhMe, and other organic solvents.

The temperature can range from 0° C. to 70° C. In certain embodiments, the temperature is 60° C.

The invention provides a process for the synthesis of an array of chiral spiro diaryl diols like IV These compounds can also be transformed to other useful compounds such as chiral ligands and chiral catalysts by simple chemical steps. The enantioselectivity can generally remain without erosion.

This process represents the first catalytic enantioselective synthetic preparation of chiral spiro diaryl diol IV.

R¹, R², R³, R⁴, R⁵, R⁶, R⁷, R⁸, R⁹, R¹⁰, R¹¹, and R¹² independently represent C₁₋₁₀₀ alkyl groups, alkoxyl groups, aryl groups, halogen or hydrogen. The number of ‘n’ was chosen from 1 to 5.

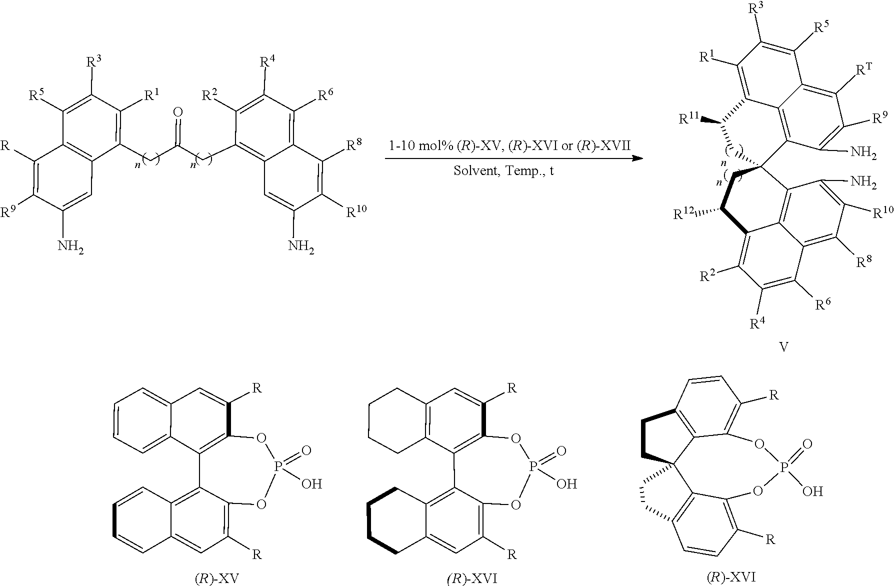

The catalyst is a chiral phosphoric acid with racemic BINOL (XV), racemic 8H-BINOL (XVI), or racemic SPINOL (XVII) backbone, in which the 3,3′-substitution group R represents hydrogen or polycyclic aromatic hydrocarbons such as substituted benzene, naphthalene, fluorene, pyrene, anthracene phenanthrene or triarylsilyl.

The catalyst here can be some other acids containing different chiral backbones.

The solvent can be chlorobenzene, PhCF₃, PhF, CCl₄, DCM, CHCl₃, PhMe, and other organic solvents.

The temperature can range from 0° C. to 70° C. In certain embodiments, the temperature is 70° C.

The invention provides a process for the synthesis of an array of chiral spiro diaryl diamines like V These compounds can also be transformed to other useful compounds such as chiral ligands and chiral catalysts by simple chemical steps. The enantioselectivity can generally remain without erosion.

This process represents the first catalytic enantioselective synthetic preparation of chiral spiro diaryl diamine V.

R¹, R², R³, R⁴, R⁵, R⁶, R⁷, R⁸, R⁹, R¹⁰, R¹¹, and R¹² independently represent C₁₋₁₀₀ alkyl groups, alkoxyl groups, aryl groups, halogen or hydrogen. The number of ‘n’ was chosen from 1 to 5.

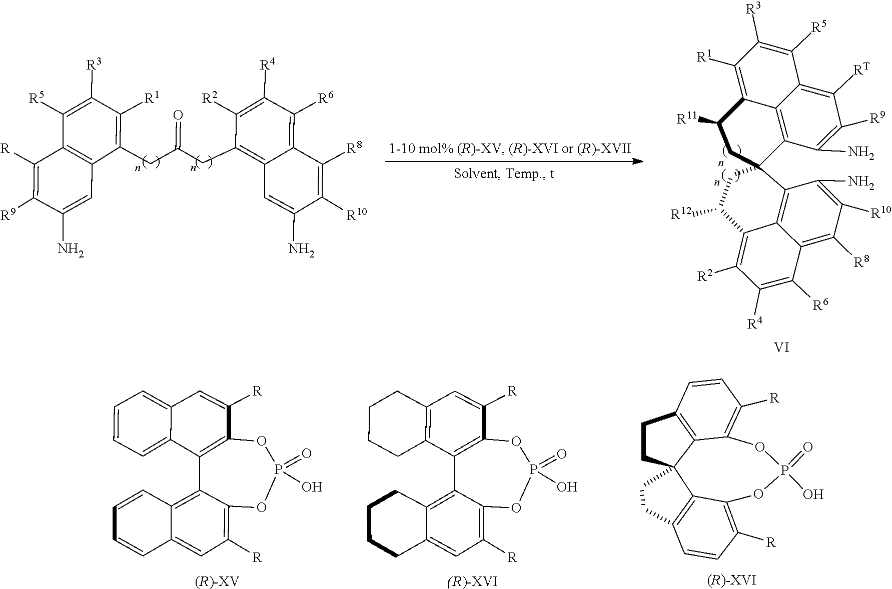

The catalyst is a chiral phosphoric acid with racemic BINOL (XV), racemic 8H-BINOL (XVI), or racemic SPINOL (XVII) backbone, in which the 3,3′-substitution group R represents hydrogen or polycyclic aromatic hydrocarbons such as substituted benzene, naphthalene, fluorene, pyrene, anthracene phenanthrene or triarylsilyl.

The catalyst here can be some other acids containing different chiral backbones.

The solvent can be chlorobenzene, PhCF₃, PhF, CCl₄, DCM, CHCl₃, PhMe, and other organic solvents.

The temperature can range from 0° C. to 70° C. In certain embodiments, the temperature is 70° C.

The invention provides a process for the synthesis of an array of chiral spiro diaryl amino naphthols like VI These compounds can also be transformed to other useful compounds such as chiral ligands and chiral catalysts by simple chemical steps. The enantioselectivity can generally remain without erosion.

This process represents the first catalytic enantioselective synthetic preparation of chiral spiro diaryl amino naphthols VI.

R¹, R², R³, R⁴, R⁵, R⁶, R⁷, R⁸, R⁹, and R¹⁰ independently represent C₁₋₁₀₀ alkyl groups, alkoxyl groups, aryl groups, halogen or hydrogen. The number of ‘n’ was chosen from 1 to 5.

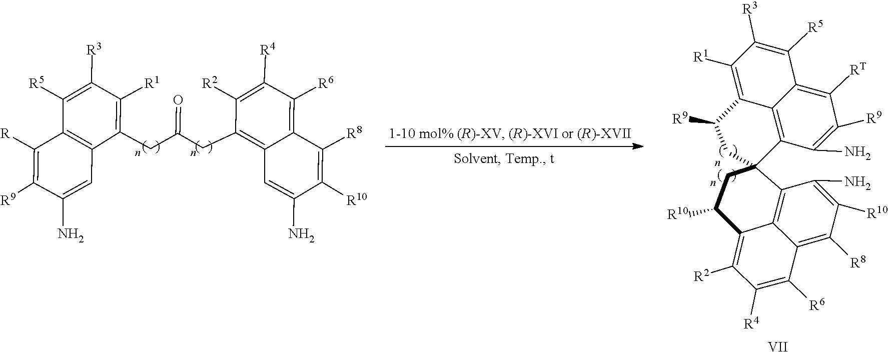

The catalyst is a Lewis acid.

The catalyst here can be some other acids containing different chiral backbones.

The solvent can be chlorobenzene, PhCF₃, PhF, CCl₄, DCM, CHCl₃, PhMe, and other organic solvents.

The temperature can range from 0° C. to 70° C. In certain embodiments, the temperature is 60° C.

The invention provides a process for the synthesis of an array of chiral spiro diaryl diindoles like VII These compounds can also be transformed to other useful compounds such as chiral ligands and chiral catalysts by simple chemical steps. The enantioselectivity can generally remain without erosion.

This process represents the first catalytic enantioselective synthetic preparation of chiral spiro diaryl diindole VII.

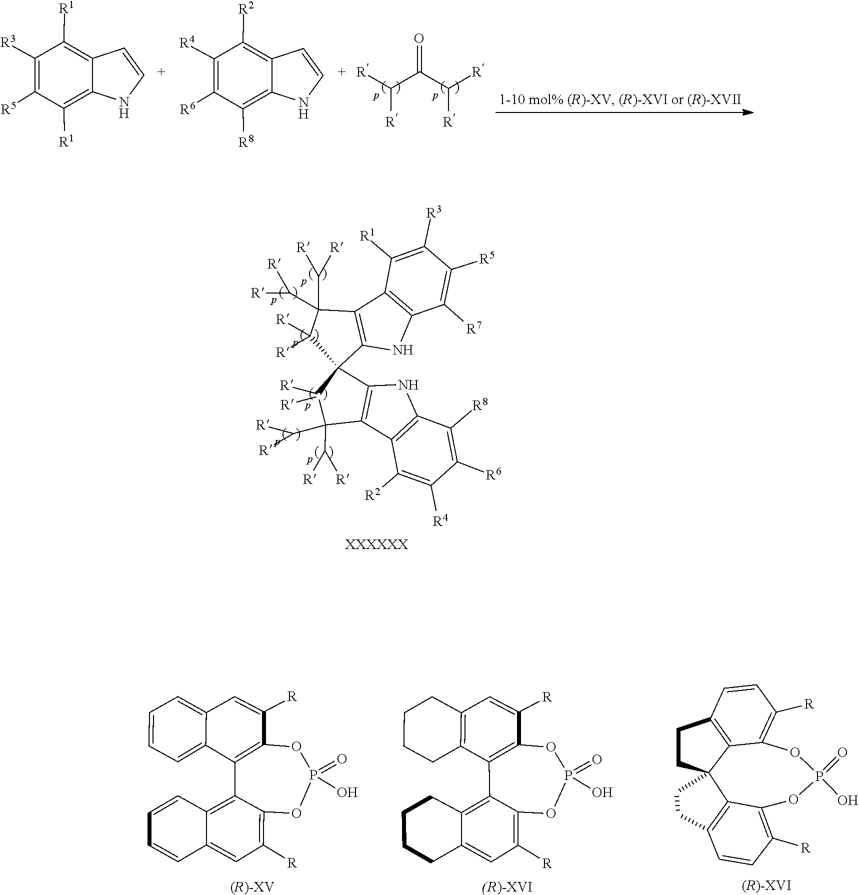

The catalyst is a chiral phosphoric acid with BINOL (XV), 8H-BINOL (XVI), or SPINOL (XVII) backbone, in which the 3,3′-substitution group R represents polycyclic aromatic hydrocarbons such as substituted benzene, naphthalene, fluorene, pyrene, anthracene phenanthrene or triarylsilyl.

The catalyst here can be some other acids containing different chiral backbones.

The solvent can be chlorobenzene, PhCF₃, PhF, CCl₄, DCM, CHCl₃, PhMe, and other organic solvents.

The temperature can range from 0° C. to 70° C. In certain embodiments, the temperature is 60° C.

The invention provides a process for the synthesis of an array of chiral spiro bis(indole) s like XXXXXX. These compounds can also be transformed to other useful compounds such as chiral ligands and chiral catalysts by simple chemical steps. The enantioselectivity can generally remain without erosion.

This process represents the first catalytic enantioselective synthetic preparation of chiral spiro bis(indole) XXXXXX.

R′, R¹, R², R³, R⁴, R⁵, R⁶, R⁷, and R⁸ for each instance independently represent C₁₋₁₀₀ alkyl groups, alkoxyl groups, aryl groups, halogen or hydrogen; and p for each instance is independently a whole number selected from 1-5.

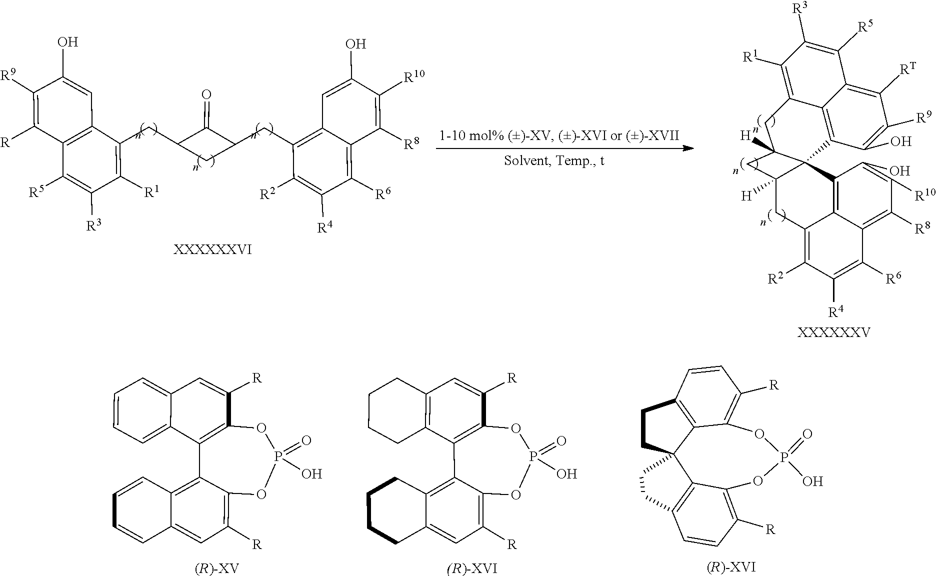

The catalyst is a chiral phosphoric acid with racemic BINOL (XV), racemic 8H-BINOL (XVI), or racemic SPINOL (XVII) backbone, in which the 3,3′-substitution group R represents hydrogen or polycyclic aromatic hydrocarbons such as substituted benzene, naphthalene, fluorene, pyrene, anthracene phenanthrene or triarylsilyl.

The catalyst here can be some other acids containing different chiral backbones.

The solvent can be chlorobenzene, PhCF₃, PhF, CCl₄, DCM, CHCl₃, PhMe, and other organic solvents.

The temperature can range from 0° C. to 70° C. In certain embodiments, the temperature is 70° C.

The invention provides a process for the synthesis of an array of chiral cycloalkane-fused spirocyclic diol like XXXXXXV. These compounds can also be transformed to other useful compounds such as chiral ligands and chiral catalysts by simple chemical steps. The enantioselectivity can generally remain without erosion.

This process represents the first catalytic enantioselective synthetic preparation of chiral cycloalkane-fused spirocyclic diol XXXXXXV.

R¹, R², R³, R⁴, R⁵, R⁶, R⁷, R⁸, R⁹, and R¹⁰ independently represent C₁₋₁₀₀ alkyl groups, alkoxyl groups, aryl groups, halogen or hydrogen; and n for each instance is independently a whole number selected from 1-5.

### Examples

Flash column chromatography was performed over silica gel (200-300 mesh) purchased from Qindao Puke Co., China. All air or moisture sensitive reactions were conducted in oven-dried glassware under nitrogen atmosphere using anhydrous solvents. Anhydrous solvents were purified by the Innovative® solvent purification system. Chemicals were purchased from commercial suppliers and used without further purification unless otherwise stated. ¹H, ¹³C, ¹⁹F, and ³¹P NMR spectra were collected on a Bruker AV 400 MHz NMR spectrometer using residue solvent peaks as an internal standard (¹H NMR:CDCl₃ at 7.26 ppm, acetone-d₆ at 2.05 ppm, MeOD-d₄ at 3.31 ppm; ¹³C NMR:CDCl₃ at 77.00 ppm, and acetone-d₆ at 29.84 ppm, MeOD-d₄ at 49.00 ppm). Data for ¹H NMR are recorded as follows: chemical shift (δ, ppm), multiplicity (s=singlet; d=doublet; t=triplet; q=quarter; p=pentet; sept=septet; m=multiplet; br=broad), coupling constant (Hz), integration. Mass spectra were collected on an Agilent GC/MS 5975C system, a MALDI Micro MX mass spectrometer, or an API QSTAR XL System. IR spectra were recorded on Bruker TENSOR 27 spectrometer and reported in terms of frequency of absorption (cm⁻¹). Optical rotations were measured on JASCO P-2000 polarimeter with [α]^{D} values reported in degrees; concentration (c) is in 10 mg/mL. The enantiomeric excess values were determined by chiral HPLC using an Agilent 1200 LC instrument with Daicel CHIRALPAK® AD-H, IC-H, AS-H, or CHIRALCEL® OD-H columns.

### Synthesis of Chiral Spiro Diamines I

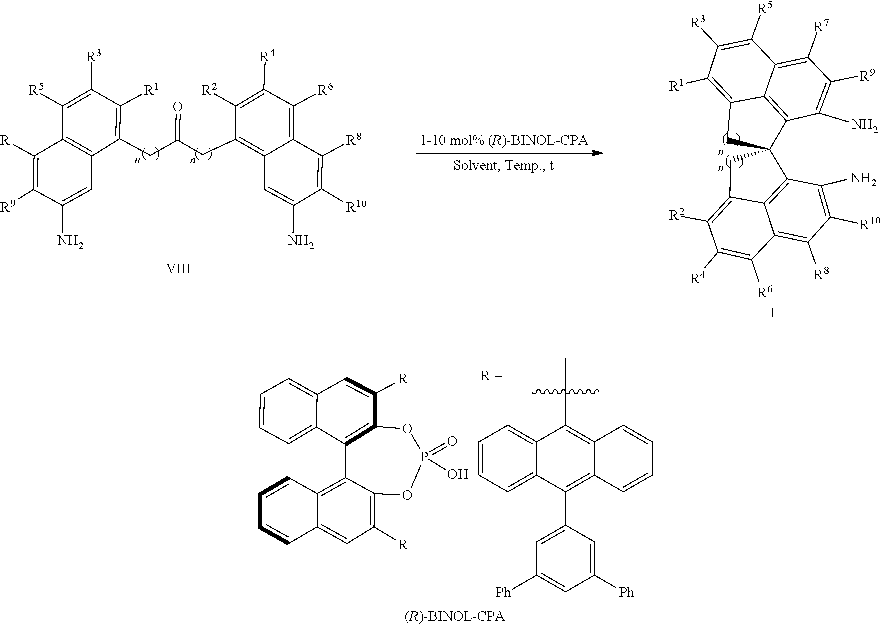

General Procedure: Ketone VIII (6.5 mmol) and (R)-BINOL-CPA (1-10 mol %) was dissolved in PhMe (65 mL) and DCM (65 mL), and then stirred for 24 h at a particular temperature. The progress was monitored by thin layer chromatography. Upon completion (time is specified in each case), the solvent was removed by evaporation and the crude product concentrated and the residue was filtered by a short pad of silica gel with DCM as eluent to give the pure product.

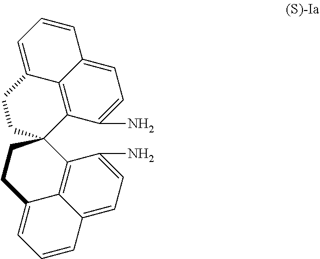

(S)-Ia was prepared as white solid from VIIIa (2.4 g, 6.5 mmol) and (R)-BINOL-CPA (150 mg, 0.13 mmol) according to the General Procedure (at 70° C.) in 90% yield (2.05 g, 90% ee). 2.05 g of (S)-Ia (90% ee) was used for recrystallization with hexane/DCM (v/v=10:1), and optically pure (R)-Ia was obtained as a white solid in 84% yield (1.75 g, >99% ee).

HPLC analysis of the product: Daicel CHIRALPAK AD-H column; 20% i-PrOH in hexanes; 1.0 mL/min; retention times: 14.6 min (major), 15.7 min (minor).

¹H NMR (400 MHZ, CDCl₃) δ 7.64-7.56 (m, 4H), 7.30-7.11 (m, 4H), 6.79 (d, J=8.7 Hz, 2H), 3.53 (s, 4H), 3.41-3.25 (m, 2H), 3.12-2.97 (m, 2H), 2.55-2.29 (m, 4H).

¹³C NMR (101 MHz, CDCl₃) δ 140.8, 133.2, 130.4, 128.9, 128.3, 126.5, 125.2, 121.9, 119.5, 117.6, 40.2, 27.9, 26.4.

HRMS (ES+) Calcd for C₂₅H₂₃N₂⁺ (M+H⁺): 351.1856, Found: 351.1860.

### Preparation of VIII-1

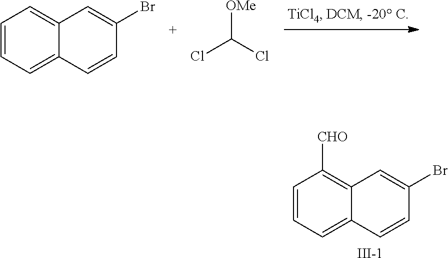

At −20° C. under N₂, to a solution of 1,1-dichlorodimethyl ether (120 mmol) in anhydrous DCM (100 mL) was added dropwise TiCl₄ (200 mmol). Then the mixture was stirred at the same temperature for 15 min, after which a solution of 2-Bromonaphthalene (100 mmol) in anhydrous DCM (50 mL) was added dropwise. The mixture was stirred at −20° C. Upon completion, the mixture was carefully poured into an aqueous solution of hydrochloric acid (2.0 M, 300 mL) at 0° C. The two layers were separated, and the aqueous layer was extracted with DCM (100 mL×3). The combined organic layers were washed with a saturated aqueous solution of NaHCO₃ (200 mL×3), brine (200 mL), dried over Na₂SO₄, and filtered. The filtrate was concentrated and purified by column and recrystallization to afford the desired product VIII-1 as a white solid (12.5 g, 60% yield).

¹H NMR (400 MHZ, Chloroform-d) δ 10.38 (s, 1H), 10.17 (d, J=2.2 Hz, 1H), 8.33 (dd, J=9.0, 2.3 Hz, 1H), 8.20 (d, J=8.3 Hz, 1H), 8.14 (dd, J=7.1, 1.3 Hz, 1H), 8.05 (d, J=9.0 Hz, 1H), 7.87 (dd, J=8.3, 7.1 Hz, 1H)

¹³C NMR (101 MHz, Chloroform-d) δ 192.6, 147.7, 138.2, 136.0, 134.7, 132.6, 129.9, 129.0, 128.6, 121.9, 120.6.

### Synthesis of VIII-3

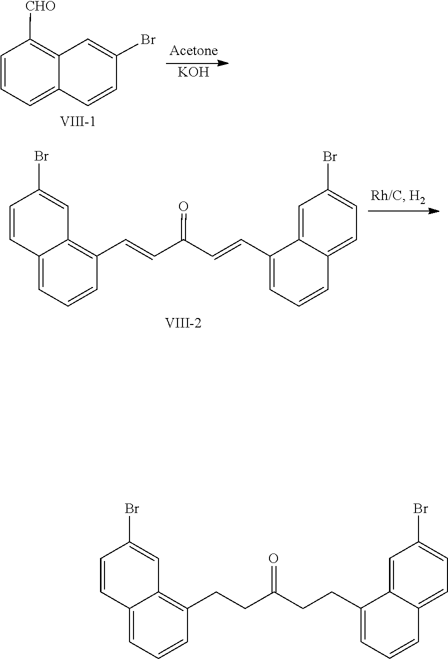

At 0° C., to a solution of 7-bromo-1-naphthaldehyde VIII-1 (2.34 g, 10.0 mmol) and acetone (290 mg, 5 mmol) in absolute EtOH (20 mL) was slowly added KOH (2.24 g, 10 mmol), which formed a yellow suspension. The mixture was stirred at the same temperature. Upon completion (˜12 h), the mixture was filtered through glass frit, and the yellow filter cake was washed with water (20 mL×2) followed by EtOH (20 mL×2). This yellow solid was dried under vacuum to afford dienone VIII-2 (2.23 g, 91% yield). This yellow solid was used directly for the next step without further purification.

Next, dienone VIII-2 (4.9 g, 10.0 mmol) was dissolved in THF (60 mL), to which was added Rh/C (490 mg, 10 wt %). The mixture was transferred into a Parr autoclave, which was flushed with hydrogen gas three times and finally pressurized to 2 bar. The mixture was stirred at room temperature for 12 h, and the hydrogen gas was released carefully in a fume hood. The mixture was filtered through a pad of celite and the filter cake was washed with EtOAc (30 mL×3). The filtrate was concentrated and the crude product was purified by recrystallization to afford the ketone VIII-3 as a white solid (3.90 g, 79% yield).

¹H NMR (400 MHZ, Chloroform-d) δ 8.10 (s, 2H), 7.75-7.62 (m, 4H), 7.58-7.51 (m, 2H), 7.41-7.34 (m, 2H), 7.34-7.27 (m, 2H), 3.30 (t, J=7.6 Hz, 4H), 2.82 (t, J=7.6 Hz, 4H).

¹³C NMR (101 MHZ, Chloroform-d) δ 208.6, 136.1, 132.7, 132.2, 130.5, 128.9, 127.0, 126.8, 126.0, 125.7, 43.4, 26.4.

HRMS (ES+) Calcd for C₂₅H₂₀Br₂NaO (M+Na⁺): 516.9733, Found: 516.9777.

### Synthesis of VIII-4

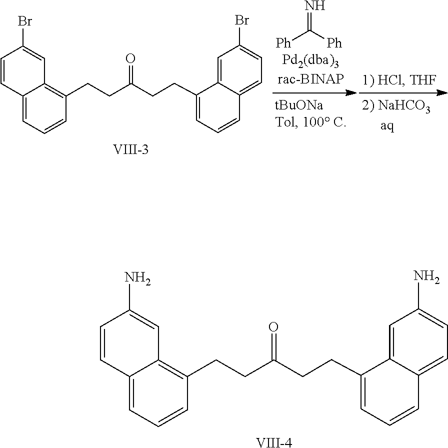

At room temperature, a 100-mL round-bottom flask equipped with a stir bar was charged with VIII-3 (4.94 g, 10.0 mmol), benzophenone imine (4.53 g, 25.0 mmol), Pd₂(dba)₃ (229 mg, 0.25 mol), rac-BINAP (376 mg, 0.6 mmol), tBuONa (2.88 g, 30 mmol), and toluene (50 mL). The flak was evacuated and refilled with N₂ for 5 times. Then, the mixture was stirred under N₂ at 110° C. for 24 h. After cooling to room temperature, the mixture was evaporated and purified by silica gel flash chromatography (eluent: hexanes/EtOAc=10:1) to afford the product as a yellow foam.

The yellow foam was dissolved in THF (50 mL) and 1.0 M HCl (50 mL) was added. The mixture was stirred vigorously at room temperature. Upon completion (˜1 h), the mixture was diluted with EA (50 mL) and poured into saturated NaHCO₃ aq carefully at 0° C. The mixture was extracted with EA (30 mL×3), washed with brine, dried over Na₂SO₄ and filtered. The filtrate was concentrated and purified by column (eluent: DCM/EtOAc=5:1) to afford the desired product VIII-4 as a yellow foam (3.2 g, 87% yield).

¹H NMR (400 MHZ, Chloroform-d) δ 7.66 (d, J=8.7 Hz, 2H), 7.58 (d, J=8.0 Hz, 2H), 7.23-7.18 (m, 2H), 7.18-7.11 (m, 2H), 7.05 (s, 1H), 6.92 (d, J=8.7 Hz, 1H), 3.74 (s, 4H), 3.24 (t, J=7.8 Hz, 4H), 2.80 (t, J=7.8 Hz, 4H).

¹³C NMR (101 MHz, Chloroform-d) δ 210.1, 144.4, 134.5, 132.9, 130.2, 128.1, 126.7, 126.3, 122.0, 117.7, 104.5, 43.4, 26.9.

HRMS (ES+) Calcd for C₂₅H₂₄N₂NaO (M+Na⁺): 391.1781, Found: 391.1782.

### Synthesis of Chiral Spiro Amino Naphthols II

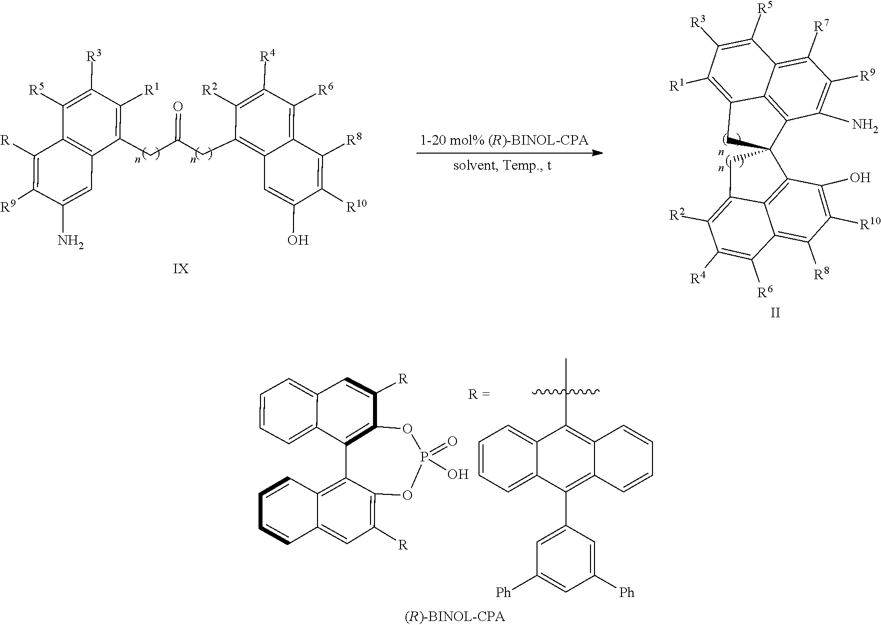

General Procedure: Ketone IX (3 mmol) and (R)-BINOL-CPA (1-10 mol %) was dissolved in o-xylene (30 mL), and then stirred for 12 h at a particular temperature. The progress was monitored by thin layer chromatography. Upon completion (time is specified in each case), the solvent was removed by evaporation and the crude product concentrated and the residue was filtered by a short pad of silica gel with DCM as eluent to give the pure product.

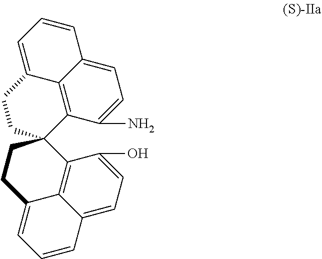

(S)-IIa was prepared as white solid from IXa (1.1 g, 3.0 mmol) and (R)-BINOL-CPA (347 mg, 0.3 mmol) according to the General Procedure (at 70° C.) in 92% yield (969 mg, 95% ee). 969 mg of (S)-IIa (95% ee) was used for recrystallization with hexane/Tol (v/v=2:1), and optically pure(S)-IIa was obtained as a white solid in 86% yield (831 mg, >99% ee).

HPLC analysis of the product: Daicel CHIRALPAK AD-H column; 20% i-PrOH in hexanes; 1.0 mL/min; retention times: 12.0 min (minor), 15.9 min (major).

¹H NMR (400 MHZ, CDCl₃) δ 7.73-7.49 (m, 4H), 7.33-7.17 (m, 4H), 7.02 (d, J=8.8 Hz, 1H), 6.76 (d, J=8.7 Hz, 1H), 5.33 (s, 1H), 3.58 (s, 2H), 3.36-3.19 (m, 2H), 3.12-2.94 (m, 2H), 2.51-2.38 (m, 2H), 2.36-2.14 (m, 2H).

¹³C NMR (101 MHZ, CDCl₃) δ 151.4, 141.6, 133.3, 132.9, 130.5, 129.9, 129.7, 129.6, 129.0, 128.9, 126.7, 126.6, 125.7, 125.3, 123.1, 122.2, 120.3, 119.5, 118.6, 113.9, 39.5, 30.8, 27.4, 26.3, 26.2.

### Synthesis of IX-2

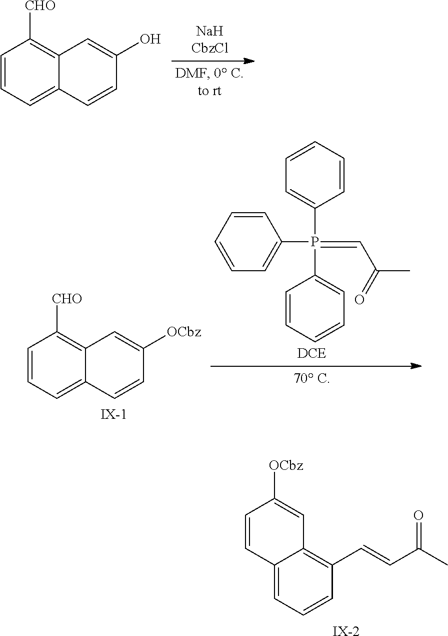

At 0° C. under N₂, 7-hydroxy-1-naphthaldehyde (1.72 g, 10 mmol) was dissolved in anhydrous DMF (10 mL) and NaH (60% dispersion in mineral oil, 600 mg, 15 mmol) was added portionwise. The mixture was stirred at room temperature for 1 h, after which CbzCl (3.4 mL, 15 mmol) was added dropwise at 0° C. The mixture was stirred for additional 1 h at room temperature. Subsequently, the reaction mixture was cooled to 0° C. and quenched by dropwise addition of a saturated aqueous NH₄Cl solution (10 mL) and H₂O (20 mL). The resulting mixture was extracted with EA (30 mL×3). The combined organic layers were washed with brine, dried over Na₂SO₄, and concentrated. The residue was purified by silica gel flash chromatography (eluent: hexanes/DCM=1:1) to afford the product IX-1 as a white solid in 96% yield (2.94 g, 9.6 mmol).

To a solution of IX-1 (2.94 g, 9.6 mmol) in DCE (10 mL) was added (Acetylmethylene)triphenylphosphorane (2.0 eq) portionwise. Then the mixture was stirred at 70° C. Upon completion, the mixture was concentrated and purified by column (eluent: hexanes/DCM=1:1) to afford the desired product IX-4 as a white solid (3.2 g, 95% yield).

¹H NMR (400 MHZ, Chloroform-d) δ 8.21 (d, J=16.0 Hz, 1H), 7.95 (s, 1H), 7.90 (d, J=8.7 Hz, 2H), 7.78 (d, J=7.2 Hz, 1H), 7.51-7.45 (m, 3H), 7.45-7.34 (m, 4H), 6.79 (d, J=16.0 Hz, 1H), 5.33 (s, 2H), 2.46 (s, 3H).

¹³C NMR (101 MHZ, Chloroform-d) δ 198.0, 153.6, 149.5, 139.4, 134.5, 131.9, 131.7, 131.6, 130.44, 130.38, 129.8, 128.8, 128.7, 128.6, 125.8, 125.5, 121.0, 114.1, 70.5, 27.8.

### Synthesis of IX-3

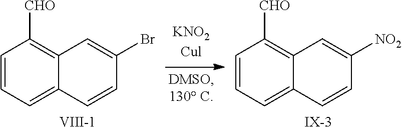

To a solution of VIII-1 (30 mmol) in DMSO (100 mL) was added KNO₂ (90 mmol) and CuI (45 mmol). Then the mixture was stirred at 130° C. for 24 h. After cooling down, saturated NH₄Cl aq. and DCM (200 mL) were added. The two layers were separated, and the aqueous layer was extracted with DCM (100 mL×3). The combined organic layers were washed with brine (200 mL), dried over Na₂SO₄, and filtered. The filtrate was concentrated and purified by column (eluent: hexanes/DCM=1:1) to afford the product IX-1 as a white solid in 63% yield (3.78 g, 18.9 mmol).

¹H NMR (400 MHZ, Chloroform-d) δ 10.38 (s, 1H), 10.17 (d, J=2.2 Hz, 1H), 8.33 (dd, J=9.0, 2.3 Hz, 1H), 8.20 (d, J=8.3 Hz, 1H), 8.14 (dd, J=7.1, 1.3 Hz, 1H), 8.05 (d, J=9.0 Hz, 1H), 7.87 (dd, J=8.3, 7.1 Hz, 1H).

¹³C NMR (101 MHz, Chloroform-d) δ 192.6, 147.7, 138.2, 136.0, 134.7, 132.6, 129.9, 129.0, 128.6, 121.9, 120.6.

HRMS (CI) Calcd for C₁₁H₇NO₃ (M): 201.0426, Found: 201.0430.

### Synthesis of IXa

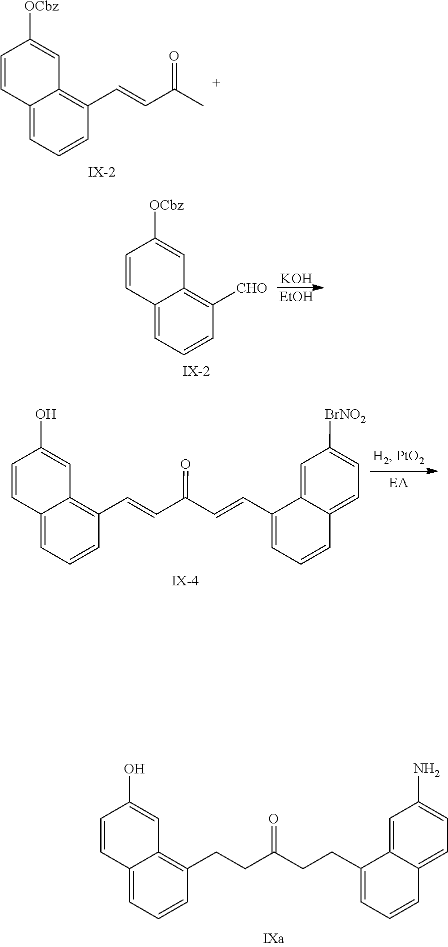

At room temperature, to a solution of IX-2 (3.46 g, 10.0 mmol) and IX-3 (2.01 g, 10 mmol) in absolute EtOH (20 mL) was slowly added KOH (4.48 g, 20 mmol), which formed a dark red suspension. The mixture was stirred at the same temperature. Upon completion (˜24 h), the mixture was carefully poured into 2.0 M HCl at 0° C. and the mixture was stirred vigorously. Then the mixture was filtered through glass frit, and the brown filter cake was washed with water (20 mL×2) followed by EtOH (20 mL×2). This brown solid purified by recrystallization and dried under vacuum to afford dienone IX-4 (6.32 g, 80% yield).

At room temperature, to a solution of IX-4 (395 mg, 1 mmol) in EA (5 mL) was added PtO₂ (40 mg, 10 wt %). The mixture was stirred at room temperature for 24 h under a H₂ atmosphere. Then the mixture was stirred at room temperature for 4 h. Upon completion, the mixture was then filtered through Celite, and the filtrate was concentrated and purified by column (eluent: hexanes/EtOAc=1:1) to afford the product IXa as a white foam in 71% yield (262 mg, 0.71 mmol).

¹H NMR (400 MHZ, Methanol-d₄) δ 7.70-7.59 (m, 1H), 7.58-7.49 (m, 2H), 7.49-7.39 (m, 1H), 7.25-7.18 (m, 1H), 7.12-6.89 (m, 7H), 3.19-3.00 (m, 4H), 2.73-2.59 (m, 4H).

¹³C NMR (101 MHZ, Methanol-d₄) δ 212.4, 156.6, 146.8, 136.3, 135.6, 134.4, 134.4, 131.5, 130.9, 130.1, 129.5, 127.62, 127.60, 127.3, 127.1, 123.6, 122.7, 119.2, 118.7, 106.2, 105.7, 44.09, 44.06, 28.0, 27.9.

### Synthesis of Chiral Spiro Bis(Indole) III

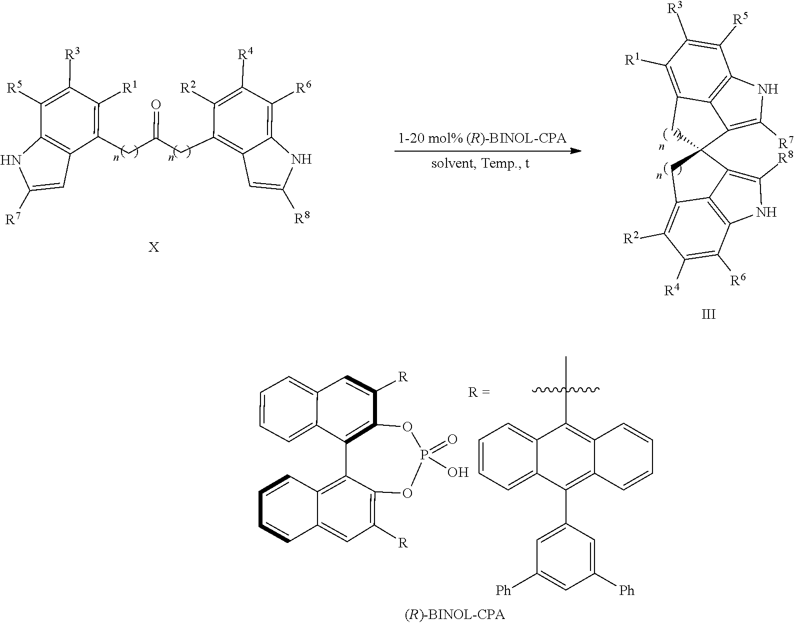

General Procedure: Ketone X (0.5 mmol) and (R)-BINOL-CPA (1-20 mol %) was dissolved in PhMe (5 mL) and DCE (5 mL), and then stirred for 5 h at a particular temperature. The progress was monitored by thin layer chromatography. Upon completion (time is specified in each case), the solvent was removed by evaporation and the crude product concentrated and the residue was filtered by a short pad of silica gel with n-hexane and DCM as eluent to give the pure product.

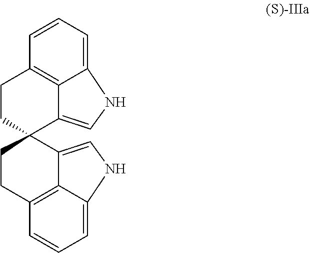

(S)-IIIa was prepared as white solid from Xa (158 mg, 0.5 mmol) and (R)-BINOL-CPA (116 mg, 0.05 mmol) according to the General Procedure (at 60° C.) in 73% yield (109 mg, 93% ee). 109 mg of (S)-Ia (93% ee) was used for recrystallization with hexane/DCM (v/v=10:1), and optically pure (R)-Ia was obtained as a white solid in 90% yield (98 mg, >99% ee).

HPLC analysis of the product: Daicel CHIRALPAK® IC-3 column; 10% i-PrOH in hexanes; 1.0 mL/min; retention times: 8.7 min (major), 9.8 min (minor).

¹H NMR (400 MHZ, CDCl₃) δ 7.77 (s, 2H), 7.25-7.07 (m, 4H), 7.04-6.88 (m, 2H), 6.66 (d, J=2.1 Hz, 2H), 3.22-3.11 (m, 2H), 3.10-2.98 (m, 2H), 2.40-2.28 (m, 2H), 2.23-2.07 (m, 2H).

¹³C NMR (101 MHz, CDCl₃) δ 133.9, 131.6, 126.4, 122.5, 122.1, 117.5, 116.0, 108.0, 36.6, 35.0, 24.7.

HRMS (ES−) Calcd for C₂₁H₁₈N₂ (M−H⁺): 297.1397, Found: 297.1393.

### Synthesis of Chiral X-1

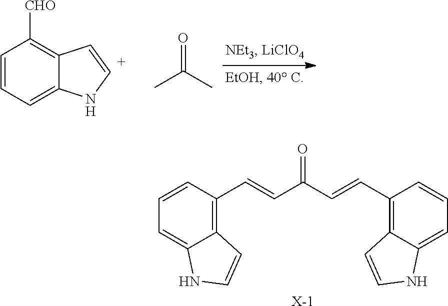

To a solution of LiClO₄ (2.2 g, 20 mmol), indole-4-carboxaldehyde (22 mmol), and acetone (10 mmol) in EtOH (3 ml), Et₃N (0.28 mL, 22 mmol) was added. The reaction mixture was stirred at room temperature and monitored by TLC. Upon completion (˜12 h), the dark red mixture was carefully poured into the water at room temperature. The organic layer was separated, and the aqueous layer was extracted with EtOAc (50 mL×3) and dried over anhydrous Na₂SO₄. The filtrate was concentrated, and the product X-1 was purified by silica gel flash chromatography (eluent: hexanes/acetone=10:1→4:1).

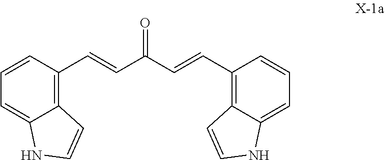 [SMILES: O=C(/C=C/c1cccc2[nH]ccc12)/C=C/c1cccc2[nH]ccc12]

X-1a was prepared according to General Procedure as an orange solid in 85% yield (2.6 g).

¹H NMR (400 MHZ, acetone-d₆) δ 10.62 (s, 2H), 8.24 (d, J=16.0 Hz, 2H), 7.59-7.49 (m, 8H), 7.22 (t, J=7.7 Hz, 2H), 7.02-6.98 (m, 2H).

¹³C NMR (101 MHZ, acetone-d₆) δ 189.2, 142.4, 137.8, 128.3, 127.6, 127.4, 126.8, 122.2, 121.5, 114.7, 101.3.

HRMS (ES−) Calcd for C₂₁H₁₆N₂O (M−H⁺): 311.1190, Found: 311.1170.

### Synthesis of X

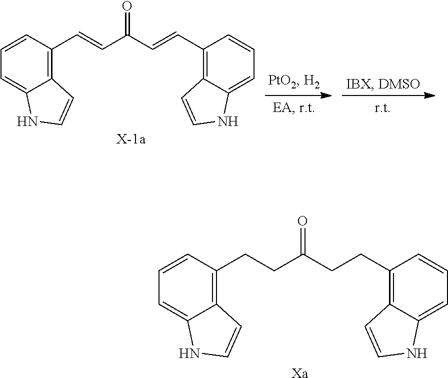 [SMILES: O=C(/C=C/c1cccc2[nH]ccc12)/C=C/c1cccc2[nH]ccc12 ; "X-1a" ; O=C(CCc1cccc2[nH]ccc12)CCc1cccc2[nH]ccc12 ; "Xa"]

To a solution of X-1a (936 mg, 3 mmol) and PtO₂ (10% wt) in EtOAc (10 mL) was added PtO₂ (93.6 mg, 10 wt %). The mixture was stirred at room temperature under H₂ atmosphere for 24 h. After the reaction was completed, the mixture was filtered through a pad of celite, and the filter cake was washed with EtOAc (30 mL×3). The filtrate was concentrated. Subsequently, IBX (840 mg, 3 mmol) and DMSO (10 mL) were added, and the mixture was stirred at room temperature for 1 h. Then, the reaction was quenched with H₂O (30 mL). The resulting mixture was extracted with EtOAc (20 mL×3). The combined organic layers were washed with brine, dried over Na₂SO₄, and concentrated. The residue was purified by silica gel flash chromatography (eluent: hexanes/acetone=10:1)

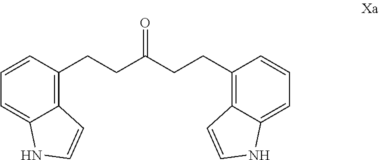 [SMILES: O=C(CCc1cccc2[nH]ccc12)CCc1cccc2[nH]ccc12]

Xa was prepared according to General Procedure as a grey solid in 70% yield (656.0 mg).

¹H NMR (400 MHZ, acetone-d₆) δ 8.20 (s, 2H), 7.26-7.24 (m, 2H), 7.20-7.17 (m, 2H), 7.15-7.09 (m, 2H), 6.90 (d, J=7.1 Hz, 2H), 6.56-6.52 (m, 2H), 3.25-3.12 (m, 4H), 2.92-2.77 (m, 4H).

¹³C NMR (101 MHZ, acetone-d₆) δ 210.4, 135.7, 133.1, 126.9, 123.8, 122.1, 119.0, 109.2, 100.7, 43.6, 27.5.

HRMS (ES−) Calcd for C₂₁H₂₀N₂O (M−H⁺): 315.1503, Found: 315.1486.

### Synthesis of Chiral Spiro Diaryl Diols IV

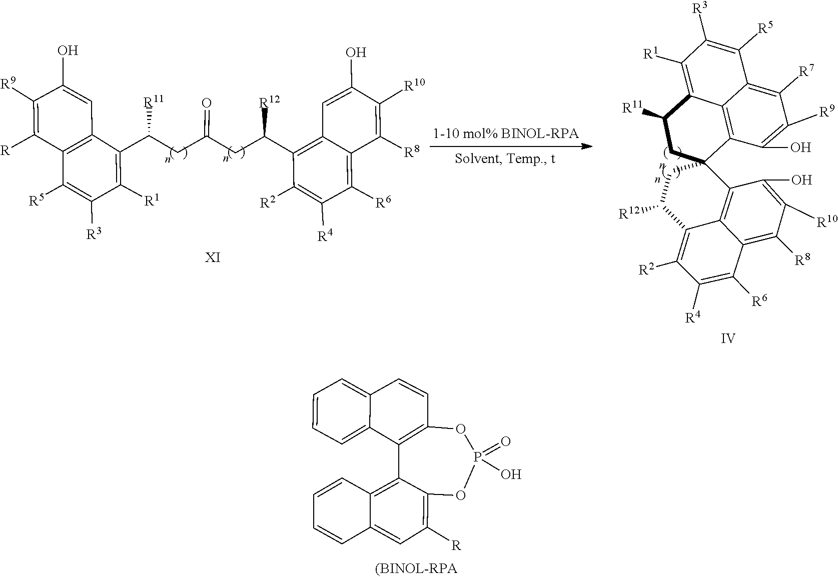

General Procedure: Ketone XI (5.0 mmol) and BINOL-RPA (1-10 mol %) was dissolved in PhMe (50 mL), and then stirred for 12 h at a particular temperature. The progress was monitored by thin layer chromatography. Upon completion (time is specified in each case), the solvent was removed by evaporation and the crude product concentrated and the residue was filtered by a short pad of silica gel with n-hexane and EtOAc as eluent to give the pure product.

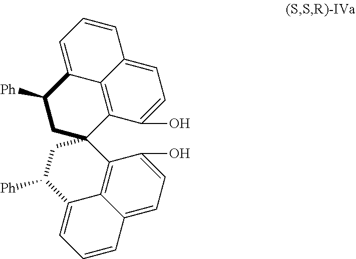

(S,S,R)-IVa was prepared as white solid from (S,S)-XIa (>99% ee, 2.61 g, 5.0 mmol) and BINOL-RPA (170 mg, 0.5 mmol) according to the General Procedure (at 60° C.) in 72% yield (1.81 g, 99% ee).

HPLC analysis of the product: Daicel CHIRALPAK AD-H column; 20% i-PrOH in hexanes; 1.0 mL/min; retention times: 14.6 min (major), 15.7 min (minor).

¹H NMR (400 MHZ, CDCl₃) δ 7.64-7.56 (m, 4H), 7.30-7.11 (m, 4H), 6.79 (d, J=8.7 Hz, 2H), 3.53 (s, 4H), 3.41-3.25 (m, 2H), 3.12-2.97 (m, 2H), 2.55-2.29 (m, 4H).

¹³C NMR (101 MHZ, CDCl₃) δ 140.8, 133.2, 130.4, 128.9, 128.3, 126.5, 125.2, 121.9, 119.5, 117.6, 40.2, 27.9, 26.4.

HRMS (ES+) Calcd for C₂₅H₂₃N₂⁺ (M+H⁺): 351.1856, Found: 351.1860.

### Synthesis of XI-2

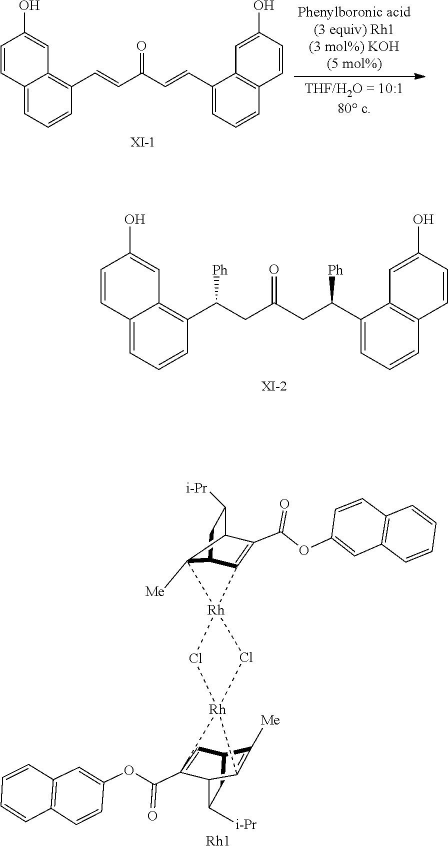

Rh1 (141 mg, 0.15 mmol), XI-1 (1.83 g, 5.0 mmol) and PhB(OH)₂ (1.83 g, 15.0 mmol) were placed in an oven-dried Schlenk tube under nitrogen. THF (40 mL) and KOH (0.0625 M aq. solution, 4 mL) were added successively, and the mixture was stirred at 80° C. for 48 h. Upon completion, the reaction mixture was diluted with EtOAc (10 mL) and water (10 mL). The layers were separated and the aqueous layer was extracted again with EtOAc for two more times (5 mL×2). The combined organic layers were dried over anhydrous Na₂SO₄, filtered, and concentrated. The residue was purified by silica gel flash column chromatography (eluent: n-hexane/EtOAc=5:1→3:1) to give the desired product XI-2 as a yellow solid in 84% yield (2.19 g, 99% ee).

HPLC analysis of the product: Daicel CHIRALPAK® ID-3 column; 30% i-PrOH in hexanes; 1.0 mL/min; retention times: 9.8 min (minor), 24.7 min (major).

¹H NMR (400 MHZ, CDCl₃) δ 7.73 (d, J=8.8 Hz, 2H), 7.64 (d, J=8.1 Hz, 2H), 7.48 (s, 2H), 7.25-6.95 (m, 16H), 6.80 (s, 2H), 5.21 (t, J=6.7 Hz, 2H), 3.21 (dd, J=17.1, 8.4 Hz, 2H), 3.08 (dd, J=17.1, 6.0 Hz, 2H).

¹³C NMR (101 MHZ, CDCl₃) δ 154.1, 143.0, 137.8, 132.5, 130.9, 129.4, 128.5, 127.9, 127.1, 126.4, 125.0, 122.9, 117.6, 105.7, 49.9, 41.1, 29.7.

HRMS (ES+) Calcd for C₃₇H₃₁O₃ [M+H]⁺: 523.2273, Found: 523.2279.

### Synthesis of Chiral Spiro Diaryl Diamines V

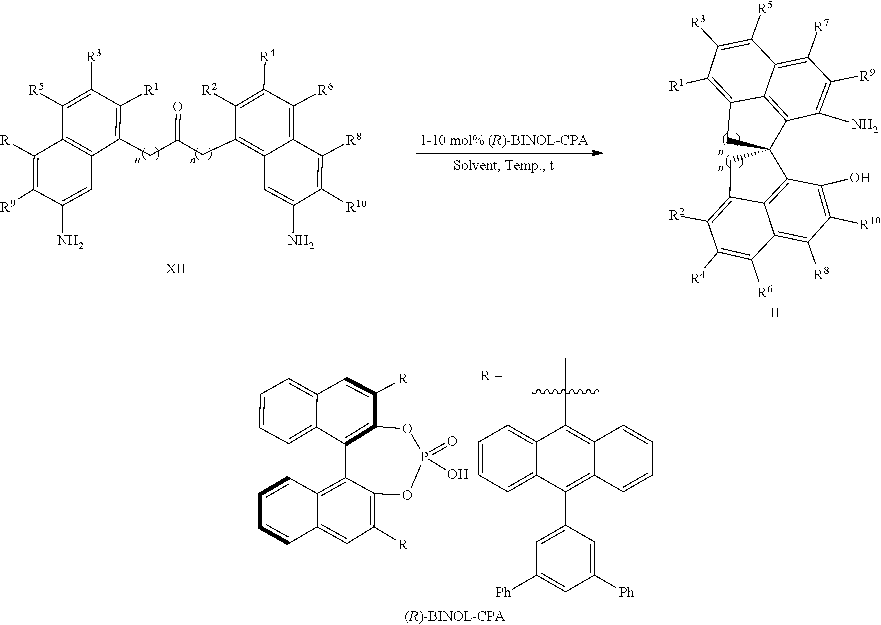

General Procedure: Ketone XII (0.2 mmol) and BINOL-RPA (1-20 mol %) was dissolved in DCM (2 mL), and then stirred for 24 h at a particular temperature. The progress was monitored by thin layer chromatography. Upon completion (time is specified in each case), the solvent was removed by evaporation and the crude product concentrated and the residue was filtered by a short pad of silica gel with n-hexane and DCM as eluent to give the pure product.

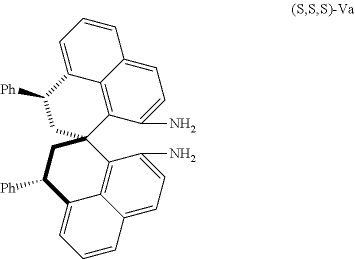

(S,S,S)-Va was prepared as white solid from (S,S)-XIIa (>99% ee, 104 mg, 0.2 mmol) and BINOL-RPA (14 mg, 0.04 mmol) according to the General Procedure (at 70° C.) in 72% yield (1.81 g, 99% ee).

¹H NMR (400 MHZ, CDCl₃) δ 7.70-7.62 (m, 4H), 7.24-7.18 (m, 2H), 7.17-7.08 (m, 6H), 7.00 (d, J=7.0 Hz, 2H), 6.85 (d, J=8.7 Hz, 2H), 6.81-6.71 (m, 4H), 4.64 (t, J=5.9 Hz, 2H), 3.72 (s, 4H), 3.07 (dd, J=13.7, 5.7 Hz, 2H), 2.25 (dd, J=13.8, 6.2 Hz, 2H).

¹³C NMR (101 MHZ, CDCl₃) δ 146.2, 141.9, 135.5, 130.8, 129.1, 128.7, 128.6, 128.0, 126.89, 126.88, 125.5, 122.0, 120.1, 117.4, 43.3, 42.3, 41.8.

HRMS (ES−) Calcd for C₃₇H₃₉N₂ (M−H⁺): 501.2335, Found: 501.2336.

### Synthesis of XII-1

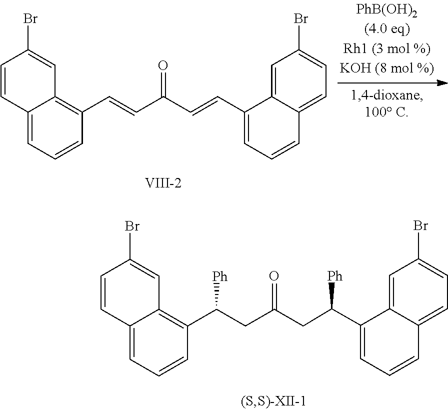

At room temperature, a 25-mL round-bottom flask equipped with a stir bar was charged with VIII-2 (294 mg, 0.6 mmol), Rh1 (16.9 mg, 0.018 mmol), aqueous solution of KOH (1.0 M, 48 μL), aryl boronic acid (2.4 mmol), and 1,4-dioxane (6.0 mL). The flask was evacuated and refilled with N₂ for 5 times. Then, the mixture was stirred under N₂ at 100° C. for 48 h. After cooling to room temperature, the mixture was concentrated and the residue was purified by silica gel flash chromatography to afford the product (S,S)—XII-1 as a white solid.

HPLC analysis of the product: Daicel CHIRALPAK AD-H column; 5% i-PrOH in hexanes; 1.0 mL/min; retention times: 21.2 min (minor), 25.9 min (major).

¹H NMR (400 MHZ, CDCl₃) δ 8.22 (s, 2H), 7.64 (d, J=8.5 Hz, 4H), 7.51-7.42 (m, 2H), 7.37-7.28 (m, 2H), 7.25-7.06 (m, 12H), 5.34-5.08 (m, 2H), 3.35-3.03 (m, 4H).

¹³C NMR (101 MHz, CDCl₃) δ 206.1, 143.1, 138.2, 132.6, 132.4, 130.4, 129.0, 128.7, 127.7, 127.2, 126.6, 126.2, 125.6, 124.9, 120.6, 49.9, 41.1.

HRMS (ES+) Calcd for C₃₇H₂₈Br₂ONa (M+Na⁺): 671.0379, Found: 671.0391.

### Synthesis of XIIa

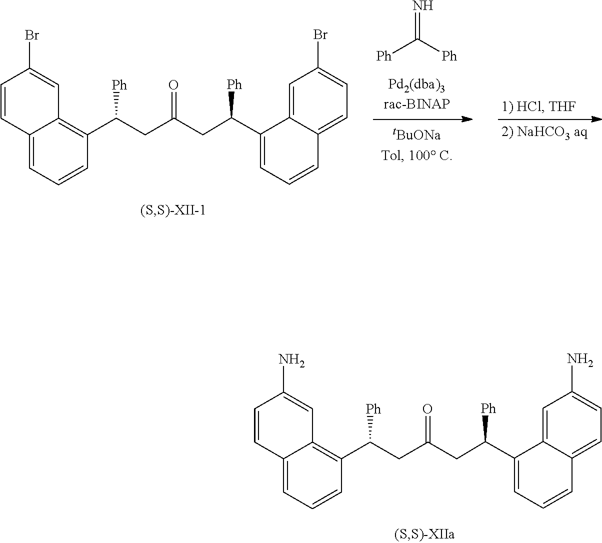 [SMILES: O=C(C[C@@H](c1ccccc1)c1cccc2ccc(Br)cc12)C[C@@H](c1ccccc1)c1cccc2ccc(Br)cc12 ; N=C(c1ccccc1)c1ccccc1 ; Nc1ccc2cccc([C@@H](CC(=O)C[C@@H](c3ccccc3)c3cccc4ccc(N)cc34)c3ccccc3)c2c1]

At room temperature, a 100-mL round-bottom flask equipped with a stir bar was charged with (S,S)—XII-1 (0.4 mmol), benzophenone imine (224 mg, 1.2 mmol), Pd₂ (dba)₃ (18 mg, 0.02 mol), rac-BINAP (31 mg, 0.05 mmol), ^{t}BuONa (144 mg, 1.5 mmol), and toluene (4.0 mL). The flask was evacuated and refilled with N₂ for 5 times. Then, the mixture was stirred under N₂ at 110° C. for 24 h. After cooling to room temperature, the mixture was evaporated and purified by silica gel flash chromatography (eluent: hexanes/EtOAc=10:1) to afford the product as a yellow foam.

The yellow foam was dissolved in THF (5.0 mL) and 1.0 M HCl (5.0 mL) was added. The mixture was stirred vigorously at room temperature. Upon completion (˜1 h), the mixture was diluted with EtOAc (20 mL) and poured into saturated NaHCO₃a.q. carefully at 0° C. The mixture was extracted with EtOAc (10 mL×3), washed with brine, dried over Na₂SO₄ and filtered. The filtrate was concentrated and purified by column (eluent: DCM/EtOAc=5:1) to afford the desired product (S,S)-XIIa as a yellow foam.

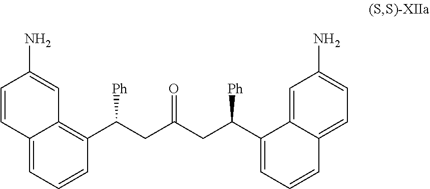 [SMILES: Nc1ccc2cccc([C@@H](CC(=O)C[C@@H](c3ccccc3)c3cccc4ccc(N)cc34)c3ccccc3)c2c1]

(S,S)-XIIa was prepared as a yellow foam from 2.7a (0.4 mmol), benzophenone imine (224 mg, 1.2 mmol), Pd₂ (dba)₃ (18 mg, 0.02 mol), rac-BINAP (31 mg, 0.05 mmol), ^{t}BuONa (144 mg, 1.5 mmol), and toluene (4.0 mL). according to the General procedure (eluent: DCM/EtOAc=5:1) in 87% yield (181 mg).

[α]_{D}²⁰: −29.4 (c=1.0, CH₂Cl₂).

¹H NMR (400 MHZ, CDCl₃) δ 7.64 (d, J=8.6 Hz, 2H), 7.58 (d, J=7.8 Hz, 2H), 7.25-7.03 (m, 16H), 6.88 (dd, J=8.7, 2.2 Hz, 2H), 5.30-5.14 (m, 2H), 3.78 (s, 4H), 3.39-3.03 (m, 4H).

¹³C NMR (101 MHZ, CDCl₃) δ 207.2, 144.4, 143.6, 136.8, 132.8, 130.1, 128.5, 128.3, 127.9, 127.0, 126.3, 124.8, 121.7, 117.7, 104.8, 49.7, 41.1.

HRMS (ES−) Calcd for C₃₇H₃₁N₂O⁻ (M−H⁺): 519.2442, Found: 519.2435.

### Synthesis of Chiral Spiro Diaryl Aminol Alcohol VI

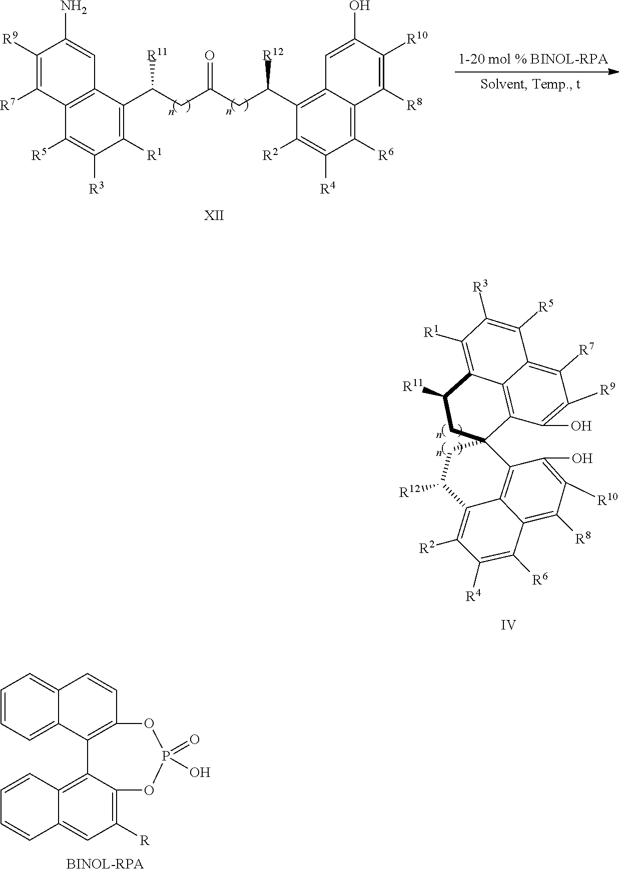

General Procedure: Ketone XIII (2.28 mmol) and BINOL-RPA (1-20 mol %) was dissolved in DCM (9 mL), and then stirred for 24 h at a particular temperature. The progress was monitored by thin layer chromatography. Upon completion (time is specified in each case), the solvent was removed by evaporation and the crude product concentrated and the residue was filtered by a short pad of silica gel with n-hexane and DCM as eluent to give the pure product.

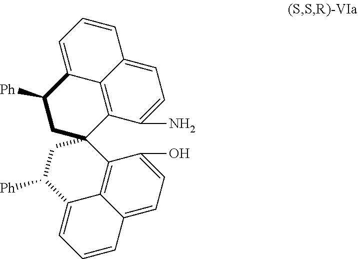

(S,S,R)-VIa was prepared as white solid from (S,S)-XIIIa (>99% ee, 1.19 g, 2.28 mmol) and BINOL-RPA (79 mg, 0.23 mmol) according to the General Procedure (at 70° C.) in 76% yield (870 mg, >99% ee).

¹H NMR (400 MHz, CDCl₃) δ 7.81-7.74 (m, 2H), 7.70 (d, J=8.7 Hz, 1H), 7.61 (d, J=8.1 Hz, 1H), 7.43-7.35 (m, 1H), 7.34-7.28 (m, 1H), 7.16-7.03 (m, 8H), 6.96-6.84 (m, 3H), 6.74-6.60 (m, 3H), 6.04 (s, 1H), 4.83 (dd, J=7.0, 2.7 Hz, 1H), 4.25 (dd, J=10.9, 4.0 Hz, 1H), 3.84 (s, 2H), 3.64 (dd, J=14.0, 6.9 Hz, 1H), 2.46 (dd, J=13.5, 4.1 Hz, 1H), 2.29 (dd, J=14.0, 2.9 Hz, 1H), 2.14-1.98 (m, 1H).

¹³C NMR (101 MHz, CDCl₃) δ 152.5, 147.4, 143.8, 142.9, 137.4, 134.1, 130.7, 130.5, 130.2, 130.0, 129.5, 128.9, 128.6, 128.1, 128.0, 127.4, 126.6, 126.1, 125.5, 125.3, 123.3, 122.4, 120.0, 119.4, 118.9, 114.4, 46.8, 43.2, 42.4, 41.4, 40.1.

HRMS (ES−) Calcd for C₃₇H₂₈NO⁻ (M−H⁺): 502.2176, Found: 502.2163.

### Synthesis of XIII-1

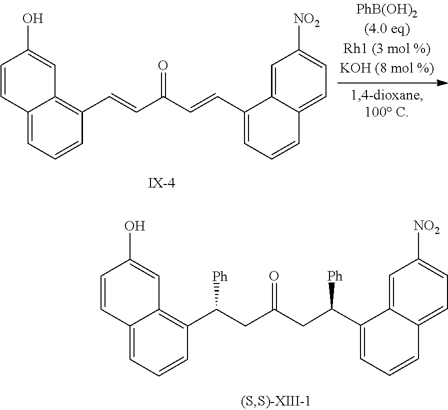

At room temperature, a 25-mL round-bottom flask equipped with a stir bar was charged with IX-4 (1.19 g, 3.0 mmol), Rh1 (56 mg, 0.06 mmol), aqueous solution of KOH (1.0 M, 0.6 mL), phenyl boronic acid (1.46 g, 12 mmol), and 1,4-dioxane (6.0 mL). The flask was evacuated and refilled with N₂ for 5 times. Then, the mixture was stirred under N₂ at 100° C. for 36 h. After cooling to room temperature, the mixture was concentrated and the residue was purified by silica gel flash chromatography to afford the product (S,S)—XIII-1 as a yellow foam in 92% yield (1.52 g, >99% ee).

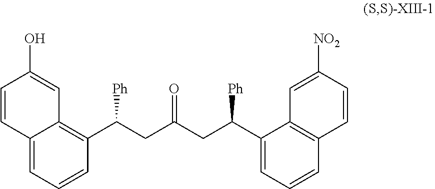 [SMILES: O=C(C[C@@H](c1ccccc1)c1cccc2ccc(O)cc12)C[C@@H](c1ccccc1)c1cccc2ccc([N+](=O)[O-])cc12]

HPLC analysis of the product: Daicel CHIRALCEL OD-3 column; 30% i-PrOH in hexanes; 1.0 mL/min; retention times: 11.9 min (minor), 37.4 min (major).

¹H NMR (400 MHZ, CDCl₃) δ 9.07 (s, 1H), 8.12 (dd, J=9.0, 2.2 Hz, 1H), 7.85 (d, J=9.0 Hz, 1H), 7.73 (d, J=8.2 Hz, 1H), 7.67 (d, J=8.8 Hz, 1H), 7.62 (dd, J=7.0, 2.3 Hz, 1H), 7.46 (t, J=7.8 Hz, 1H), 7.37 (d, J=2.4 Hz, 1H), 7.25-7.09 (m, 12H), 7.00 (dd, J=8.8, 2.4 Hz, 1H), 6.07 (s, 1H), 5.36 (t, J=7.3 Hz, 1H), 5.22 (t, J=7.4 Hz, 1H), 3.42-3.08 (m, 4H).

¹³C NMR (101 MHZ, CDCl₃) δ 207.1, 154.0, 145.4, 143.1, 143.0, 141.4, 137.1, 136.6, 132.6, 130.7, 130.3, 130.2, 129.3, 129.1, 128.8, 128.6, 127.9, 127.6, 127.2, 127.1, 126.8, 126.5, 125.5, 124.7, 122.7, 120.9, 118.9, 117.5, 105.9, 49.9, 49.7, 41.7, 41.3.

HRMS (ES+) Calcd for C₃₇H₂₉NNaO₄⁺ (M+Na⁺): 574.1989, Found: 574.1998.

### Synthesis of XIIIa

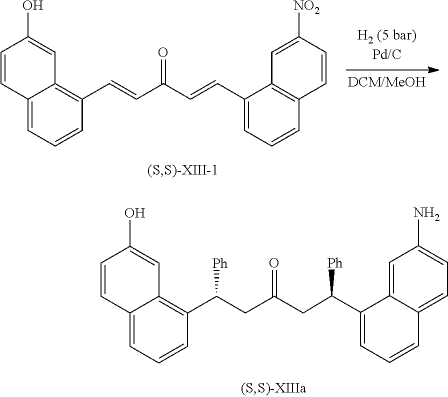 [SMILES: O=C(/C=C/c1cccc2ccc(O)cc12)/C=C/c1cccc2ccc([N+](=O)[O-])cc12 ; "(S,S)-XIII-1" ; Nc1ccc2cccc([C@@H](CC(=O)C[C@@H](c3ccccc3)c3cccc4ccc(O)cc34)c3ccccc3)c2c1]

Next, (S,S)—XIII-1 (1.10 g, 2.0 mmol) was dissolved in DCM (5.0 mL) and MeOH (5.0 mL), to which was added Pd/C (110 mg, 10 wt %). The mixture was transferred into a Parr autoclave, which was flushed with hydrogen gas three times and finally pressurized to 5 bar. The mixture was stirred at room temperature for 5 h, and the hydrogen gas was released carefully in a fume hood. The mixture was filtered through a pad of celite and the filter cake was washed with EtOAc (20 mL×3). The filtrate was concentrated and the crude product was purified by silica gel flash chromatography (eluent: hexanes/EtOAc=3:1) to afford the ketone (S,S)-XIIIa as a yellow foam in 93% yield (970 mg).

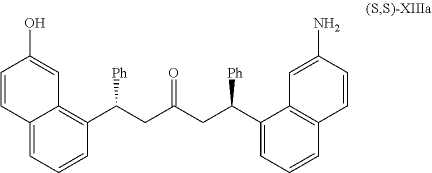 [SMILES: Nc1ccc2cccc([C@@H](CC(=O)C[C@@H](c3ccccc3)c3cccc4ccc(O)cc34)c3ccccc3)c2c1]

¹H NMR (400 MHZ, CDCl₃) δ 7.71-7.50 (m, 4H), 7.34-7.30 (m, 1H), 7.24-6.97 (m, 16H), 6.89-6.84 (m, 1H), 5.26-5.09 (m, 2H), 3.90 (s, 2H), 3.30-3.08 (m, 4H).

¹³C NMR (101 MHZ, CDCl₃) δ 208.2, 154.1, 144.2, 143.4, 143.2, 137.5, 136.9, 132.7, 132.6, 130.6, 130.2, 129.2, 128.5, 128.5, 127.9, 127.0, 127.0, 126.3, 126.3, 125.0, 124.8, 122.7, 121.9, 117.9, 117.6, 105.9, 105.3, 49.8, 49.6, 41.2, 41.2.

HRMS (ES+) Calcd for C₃₇H₃₁NNaO₂⁺ (M+Na⁺): 544.2250, Found: 544.2247.

### Synthesis of XIVa

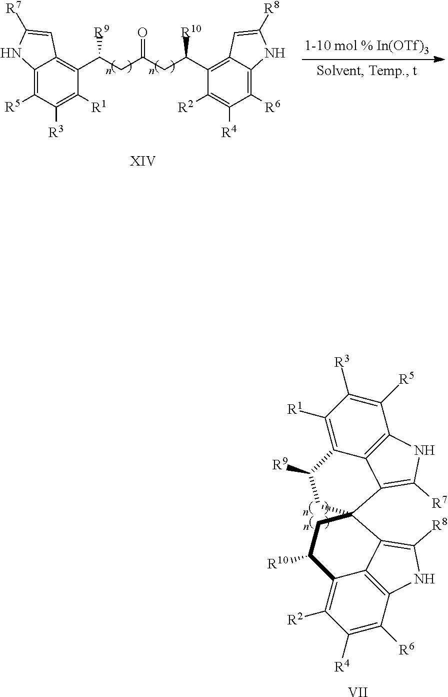

General Procedure: Ketone XIV (2.28 mmol) and In(OTf)₃ (1-20 mol %) was dissolved in EtOAc (3 mL), and then stirred for 24 h at a particular temperature. The progress was monitored by thin layer chromatography. Upon completion (time is specified in each case), the solvent was removed by evaporation and the crude product concentrated and the residue was filtered by a short pad of silica gel with n-hexane and EtOAc as eluent to give the pure product.

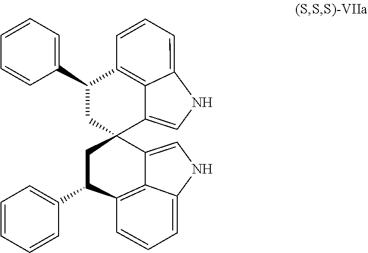

(S,S,S)-VIIa was prepared as white solid from (S,S)-XIVa (>99% ee, 1.07 g, 2.28 mmol) and In(OTf)₃ (129 mg, 0.23 mmol) according to the General Procedure (at 60° C.) in 78% yield (800 mg, 99% ee).

HPLC analysis of the product: Daicel CHIRALCEL® OD-3 column; 10% i-PrOH in hexanes; 1.0 mL/min; retention times: 18.4 min (minor), 39.7 min (major).

¹H NMR (400 MHZ, DMSO) δ 10.78 (s, 2H), 7.34-7.26 (m, 8H), 7.23-7.15 (m, 6H), 7.04-6.97 (m, 2H), 6.46-6.35 (m, 2H), 4.91-4.77 (m, 2H), 3.47-3.40 (m, 2H), 2.34 (d, J=6.7 Hz, 4H).

¹³C NMR (101 MHz, DMSO) δ 145.0, 133.6, 133.5, 128.6, 128.3, 126.2, 125.6, 121.6, 121.1, 118.1, 115.3, 108.6, 48.4, 43.0, 35.8.

HRMS (ES−) Calcd for C₃₃H₂₆N₂ (M−H⁺): 449.2023, Found: 449.2020.

### Synthesis of XIV

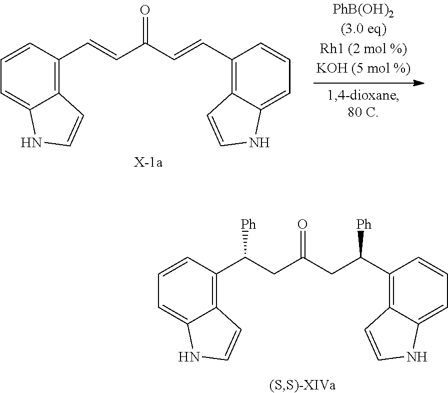 [SMILES: O=C(/C=C/c1cccc2[nH]ccc12)/C=C/c1cccc2[nH]ccc12 ; "X-1a" ; O=C(C[C@@H](c1ccccc1)c1cccc2[nH]ccc12)C[C@@H](c1ccccc1)c1cccc2[nH]ccc12 ; "(S,S)-XIVa"]

Rh1 (2 mol %, 4 mol % Rh), X-1a (0.60 mmol), and PhB(OH)₂ (1.8 mmol) were placed in a 10-mL vial under nitrogen. 1,4-dioxane (4 mL), aqueous solution of KOH (5 mol %, 1.7 mg), H₂O (0.4 mL), and the mixture was stirred at 80° C. for 24 h. Upon completion, the reaction mixture was diluted with water (5.0 mL) and EtOAc (5.0 mL×3). The organic layer was separated, and the aqueous layer was extracted with EtOAc (20 mL×3) and dried over anhydrous Na₂SO₄. The filtrate was concentrated, and the product (S,S)-XIVa was purified by silica gel flash chromatography (eluent: hexanes/EA=4:1).

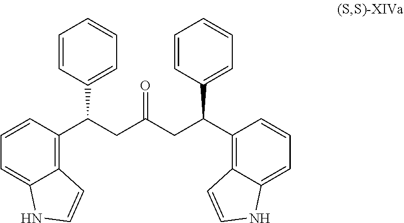 [SMILES: O=C(C[C@@H](c1ccccc1)c1cccc2[nH]ccc12)C[C@@H](c1ccccc1)c1cccc2[nH]ccc12]

(S,S)-XIVa was prepared according to the General Procedure as a pale yellow solid (eluent: hexanes/EtOAc=4:1) in 96% yield (269.6 mg, 94% ee).

HPLC analysis of the product: Daicel CHIRALPAK® AD-H column; 20% i-PrOH in hexanes; 1.0 mL/min; retention times: 26.1 min (minor), 28.2 min (major).

¹H NMR (400 MHZ, CDCl₃) δ 8.09 (s, 2H), 7.26-6.95 (m, 16H), 6.85 (d, J=6.7 Hz, 2H), 6.48 (s, 2H), 5.01 (s, 2H), 3.27 (d, J=6.6 Hz, 4H).

¹³C NMR (101 MHZ, CDCl₃) δ 207.5, 143.7, 135.8, 135.7, 128.3, 127.8, 126.8, 126.1, 123.8, 121.9, 117.4, 109.6, 101.1, 49.1, 43.2.

HRMS (ES−) Calcd for C₃₃H₂₈N₂O (M−H⁺): 467.2129, Found: 467.2120.

### Synthesis of XXXXXX

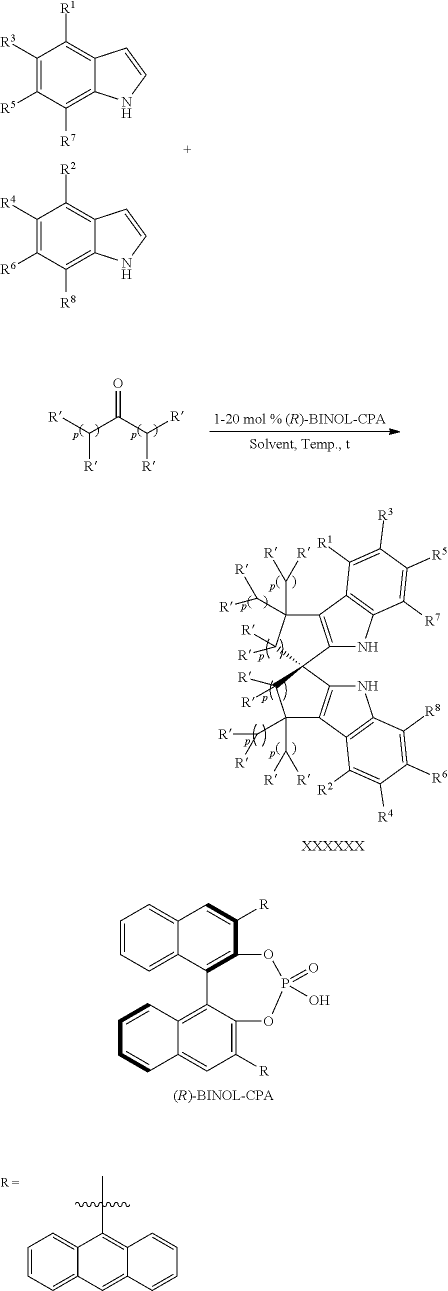

Indole (60 mmol), ketone (300 mmol), MgSO₄ (15 g), benzyl alcohol (30 mmol) and (R)-BINOL-CPA (1-20 mol %) was dissolved in CHCl₃ (150 mL), and then stirred for 48 h at a particular temperature. The progress was monitored by thin layer chromatography. Upon completion (time is specified in each case), the solvent was removed by evaporation and the crude product concentrated and the residue was filtered by a short pad of silica gel with DCM as eluent to give the pure product.

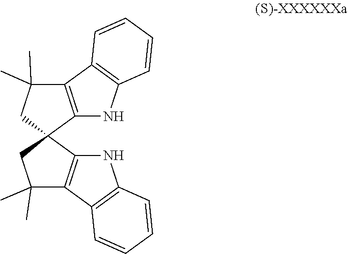

(S)-XXXXXXa was prepared as white solid from indole (7.0 g, 60 mmol), acetone (17.4 g, 300 mmol) and (R)-BINOL-CPA (3.5 g, 3 mmol) according to the General Procedure (at 70° C.) in 80% yield (8.6 g, 74% ee). 8.6 g of (S)-XXXXXXa (74% ee) was used for recrystallization with hexane/DCM (v/v=10:1), and optically pure(S)-XXXXXXa was obtained as a white solid in 56% yield (4.8 g, >99% ee).

HPLC analysis of the product: Daicel CHIRALPAK® AD-3 column; 5% i-PrOH in hexanes; 1.0 mL/min; retention times: 5.9 min (minor), 7.5 min (major).

¹H NMR (400 MHZ, CDCl₃) δ 7.66-7.59 (m, 2H), 7.58 (s, 2H), 7.25-7.19 (m, 2H), 7.18-7.07 (m, 4H), 2.81 (d, J=13.1 Hz, 2H), 2.65 (d, J=13.1 Hz, 2H), 1.63 (s, 6H), 1.54 (s, 6H).

¹³C NMR (101 MHz, CDCl₃) δ 144.4, 141.0, 127.6, 123.4, 121.1, 119.6, 118.4, 111.8, 62.1, 49.2, 39.2, 30.4, 30.3.

HRMS (ES−) Calcd for C₂₅H₂₆N₂ (M−H⁺): 353.2023, Found: 353.2003.

### Synthesis of Chiral Cycloalkane-Fused Spirocyclic Diol XXXXXXV

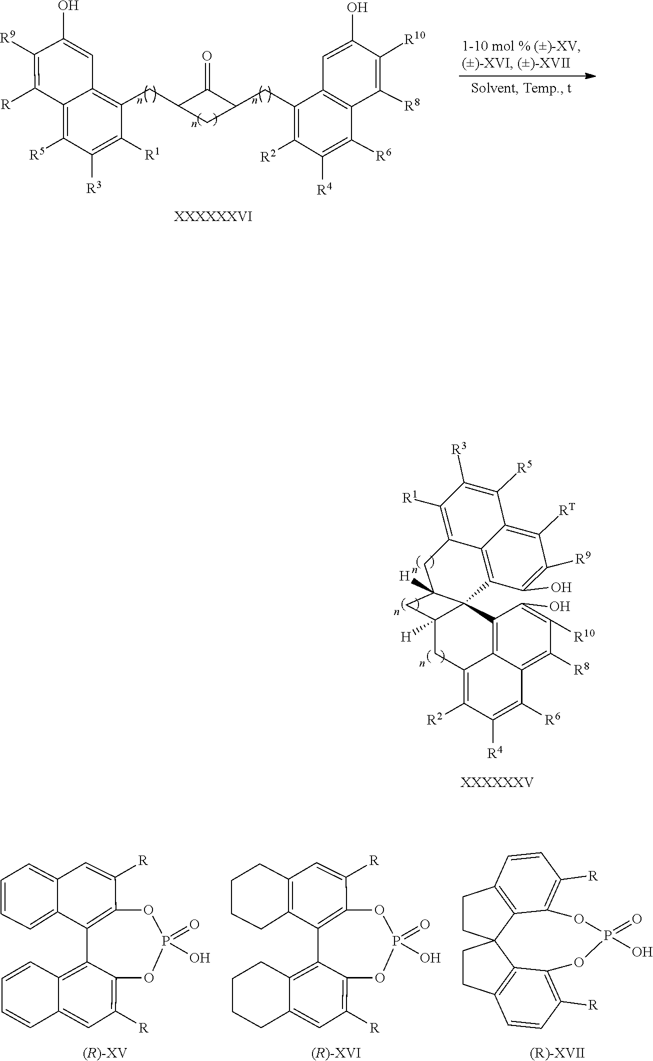

General Procedure: Ketone XXXXXXVI (0.44 mmol) and BINOL-RPA (1-20 mol %) was dissolved in DCM (4 mL), and then stirred for 24 h at a particular temperature. The progress was monitored by thin layer chromatography. Upon completion (time is specified in each case), the solvent was removed by evaporation and the crude product concentrated and the residue was filtered by a short pad of silica gel with n-hexane and DCM as eluent to give the pure product.

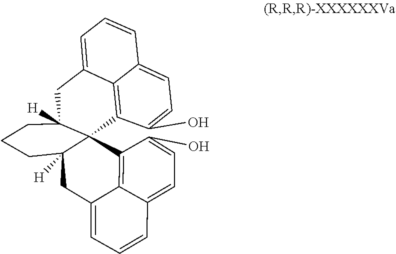

(R,R,R)-XXXXXXVa was prepared as white solid from (R,R)-XXXXXXVIa (>99% ee, 18 mg, 0.44 mmol) and BINOL-RPA (15 mg, 0.044 mmol) according to the General Procedure (at 50° C.) in 87% yield (150 mg, >99% ee).

¹H NMR (400 MHZ, CDCl₃) δ 7.73 (d, J=8.8 Hz, 2H), 7.69 (dd, J=7.3, 2.2 Hz, 2H), 7.40-7.27 (m, 4H), 6.96 (d, J=8.8 Hz, 2H), 5.25 (s, 2H), 3.34-3.04 (m, 6H), 1.52 (td, J=7.0, 3.3 Hz, 2H), 1.41 (q, J=6.1 Hz, 4H).

¹³C NMR (101 MHz, CDCl₃) δ 153.0, 134.5, 130.8, 130.0, 129.9, 126.4, 126.1, 123.6, 120.3, 119.0, 45.2, 35.0, 32.9, 27.1, 17.8.

HRMS (ES−) Calcd for C₃₇H₂₈NO⁻ (M−H⁺): 391.1704, Found: 391.1686.

### Synthesis of XXXXXXVI-1

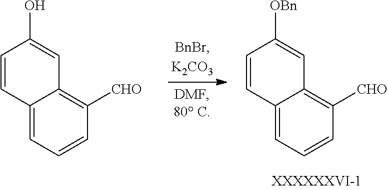 [SMILES: O=Cc1cccc2ccc(O)cc12 ; O=Cc1cccc2ccc(OCc3ccccc3)cc12]

To a solution of 7-hydroxy-1-naphthaldehyde (1.72 g, 10 mmol) in DMF (20 mL) was added benzyl bromide (2.57 g, 15 mmol) and K₂CO₃ (2.76 g, 20 mmol). The mixture was stirred at 80° C. for 2 h before cooling down to room temperature. The resulting mixture was filtered. The filtrate was concentrated and the residue was purified by silica gel flash chromatography (eluent: hexanes/DCM=2:1) to afford product XXXXXXVI-1 as a white solid (2.51 g, 96% yield).

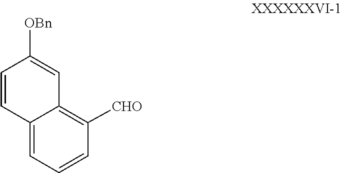 [SMILES: O=Cc1cccc2ccc(OCc3ccccc3)cc12]

¹H NMR (400 MHZ, CDCl₃) δ 10.30 (s, 1H), 8.91 (d, J=2.5 Hz, 1H), 7.99 (d, J=8.0 Hz, 1H), 7.92-7.85 (m, 1H), 7.81 (d, J=8.9 Hz, 1H), 7.61-7.52 (m, 2H), 7.50-7.30 (m, 5H), 5.27 (s, 2H).

¹³C NMR (101 MHZ, CDCl₃) δ 193.9, 159.5, 138.1, 136.4, 134.9, 131.9, 130.0, 129.8, 129.2, 128.5, 128.0, 127.8, 122.4, 119.9, 104.6, 69.9.

HRMS (CI) Calcd for C₁₈H₁₄₀₂ (M): 262.0994, Found: 262.0991.

### Synthesis of XXXXXXVI-2

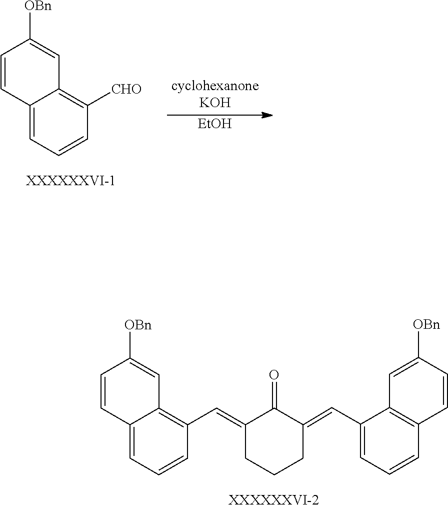

At 0° C., to a solution of XXXXXXVI-1 (2.62 g, 10 mmol) and cyclohexanone (491 mg, 5.0 mmol) in absolute EtOH (20 mL) was slowly added KOH (2.24 g, 10 mmol), which formed a yellow suspension. The mixture was stirred at the same temperature. Upon completion (˜12 h), the mixture was filtered through glass frit, and the yellow filter cake was washed with water (20 mL×2) followed by EtOH (20 mL×3). This yellow solid was dried under vacuum and purified by recrystallization to afford dienone XXXXXXVI-2 (2.64 g, 90% yield).

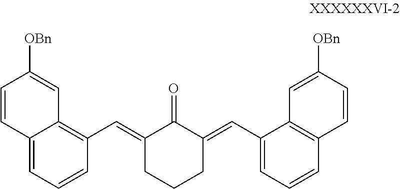 [SMILES: O=C1/C(=C/c2cccc3ccc(OCc4ccccc4)cc23)CCC/C1=C\c1cccc2ccc(OCc3ccccc3)cc12]

¹H NMR (400 MHZ, CDCl₃) δ 8.40 (s, 2H), 7.85-7.76 (m, 4H), 7.55-7.48 (m, 4H), 7.48-7.27 (m, 16H), 5.22 (s, 4H), 2.82-2.69 (m, 4H), 1.76-1.55 (m, 2H).

¹³C NMR (101 MHZ, CDCl₃) 190.2, 157.2, 138.1, 136.7, 135.3, 133.2, 131.9, 130.2, 129.1, 128.7, 128.6, 128.0, 127.7, 127.6, 122.8, 119.1, 104.7, 70.2, 28.7, 23.5.

HRMS (ES+) C₄₂H₃₄NaO₃⁺ (M+Na⁺): 609.2400, Found: 609.2402.

### Synthesis of XXXXXXVI-3

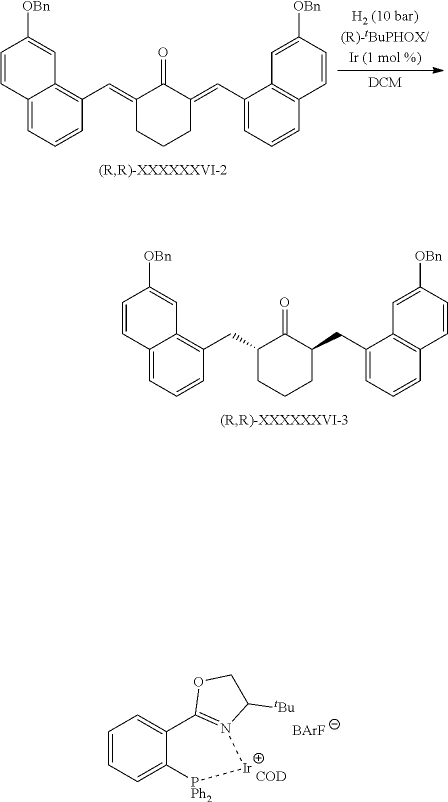

To a solution of XXXXXXVI-2 (2.34 g, 4.0 mmol) in anhydrous DCM (20 mL) was added (R)-^{t}BuPHOX/Ir (62 mg, 0.04 mmol). This vial was transferred into a Parr autoclave, which was flushed with hydrogen gas three times and finally pressurized to 10 bar. The mixture was stirred at room temperature for 12 h, and the hydrogen gas was released carefully in a fume hood. The mixture was concentrated in vacuo, and the residue was purified by silica gel flash chromatography (eluent: hexanes/DCM=2:1) to afford the pure product (R,R)—XXXXXXVI-3 as a white solid in 99% yield (2.33 g, >99% ee, >20:1 dr).

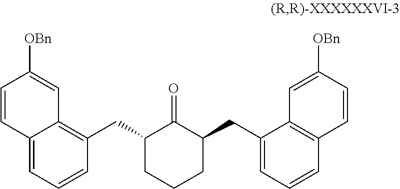 [SMILES: O=C1[C@@H](Cc2cccc3ccc(OCc4ccccc4)cc23)CCC[C@@H]1Cc1cccc2ccc(OCc3ccccc3)cc12]

¹H NMR (400 MHZ, CDCl₃) δ 7.81 (d, J=8.9 Hz, 2H), 7.72-7.66 (m, 2H), 7.54-7.49 (m, 4H), 7.42-7.36 (m, 6H), 7.35-7.21 (m, 8H), 5.31-5.14 (m, 4H), 3.67-3.51 (m, 2H), 3.03-2.76 (m, 4H), 1.94-1.78 (m, 2H), 1.77-1.69 (m, 2H), 1.69-1.58 (m, 2H).

¹³C NMR (101 MHz, CDCl₃) δ 214.7, 156.8, 136.8, 134.3, 132.8, 130.4, 129.4, 128.5, 127.9, 127.7, 127.5, 126.8, 123.1, 118.4, 103.9, 70.0, 49.1, 33.5, 31.6, 20.3.

HRMS (ES+) Calcd for C₄₂H₃₈NaO₃⁺ (M+Na⁺): 613.2713, Found: 613.2717.

### Synthesis of XXXXXXVI-4

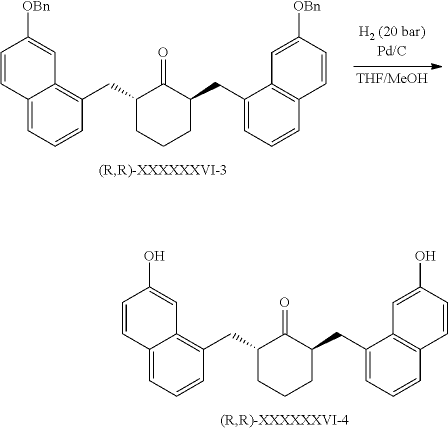

To a solution of (R,R)—XXXXXXVI-3 (1.18 g, 2.0 mmol) in THF (5.0 mL) and MeOH (5.0 mL) was added Pd/C (290 mg, 25 wt %). This vial was transferred into a Parr autoclave, which was flushed with hydrogen gas three times and finally pressurized to 20 bar. The mixture was stirred at room temperature for 24 h, and the hydrogen gas was released carefully in a fume hood. The Pd/C was recycled by centrifugation. The clear liquid was concentrated and the residue was purified by silica gel flash chromatography (eluent: hexanes/EtOAc=2:1) to afford the pure product (R,R)—XXXXXXVI-4 as a white foam in 90% yield (735 mg).

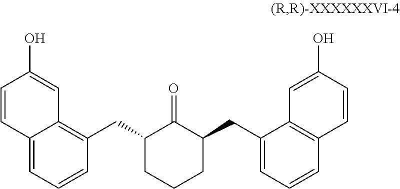 [SMILES: O=C1[C@@H](Cc2cccc3ccc(O)cc23)CCC[C@@H]1Cc1cccc2ccc(O)cc12]

¹H NMR (400 MHZ, CDCl₃) δ 8.41 (s, 2H), 7.69 (d, J=8.9 Hz, 2H), 7.60 (d, J=8.1 Hz, 2H), 7.43 (d, J=2.4 Hz, 2H), 7.24-7.11 (m, 4H), 7.07 (d, J=6.9 Hz, 2H), 3.54 (dd, J=14.0, 3.7 Hz, 2H), 3.01-2.90 (m, 2H), 2.80-2.65 (m, 2H), 1.76-1.65 (m, 2H), 1.63-1.40 (m, 4H).

¹³C NMR (101 MHz, CDCl₃) δ 219.3, 154.1, 133.2, 132.7, 130.8, 129.1, 127.7, 126.9, 122.6, 117.6, 105.7, 49.1, 33.4, 31.0, 19.8.

(ES−) Calcd for C₂₈H₂₅O₃⁻ (M−H⁺): 409.1809, Found: 409.1806.

### Synthesis of Biphosphine XVIII

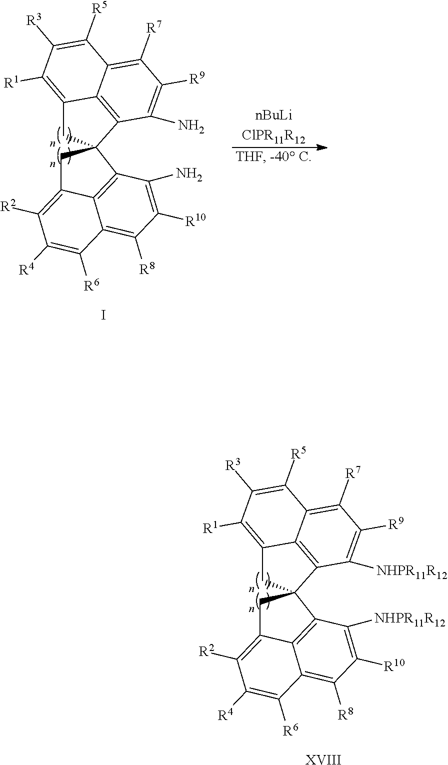

General Procedure: In a dry flask equipped with a stir bar, chiral diamine I (350 mg, 1.0 mmol) was dissolved in dry THF (10.0 mL) at −40° C. and ⁿBuLi (2.4 M in hexanes, 1.0 mL, 2.4 mmol) was added dropwise. After that the reaction solution was stirred at −40° C. for 1 h. Then ClPR₁₁R₁₂ was added at −40° C. Then the mixture was allowed to warm to room temperature. Upon completion, the mixture was quenched by NH₄Cl aq, extracted with Et₂O (20 mL×3), washed with H₂O and brine, dried over Na₂SO₄, and concentrated. The residue was subjected to silica gel flash chromatography to give the pure product XVIII.

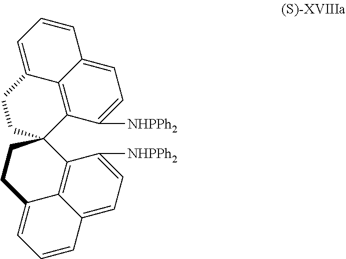

(S)-XVIIIa was prepared as white foam from(S)-Ia (>99% ee, 350.0 mg, 1.0 mmol) and chlorodiphenylphosphine (660 mg, 3.0 mmol) according to the General Procedure (eluent: hexanes/DCM=4:1) in 72% yield (539 mg).

¹H NMR (400 MHZ, CDCl₃) δ 7.67-7.49 (m, 6H), 7.32-7.07 (m, 12H), 7.04-6.87 (m, 8H), 6.78-6.61 (m, 4H), 4.62 (d, J=8.5 Hz, 2H), 3.27-3.09 (m, 2H), 2.90-2.65 (m, 2H), 2.33-2.21 (m, 2H), 2.19-2.03 (m, 2H).

¹³C NMR (101 MHZ, CDCl₃) δ 141.4, 141.2, 139.9, 139.81, 139.80, 139.7, 132.7, 131.1, 130.9, 130.3, 130.1, 129.8, 129.1, 128.8, 128.5, 128.4, 128.3, 128.31, 128.25, 128.2, 128.1, 126.6, 125.2, 122.3, 120.22, 120.19, 118.2, 118.0, 40.1, 28.7, 26.2.

³¹P NMR (162 MHZ, CDCl₃) δ 26.09.

HRMS (CI+) Calcd for C₄₉H₄₁N₂P₂⁺ (M+H⁺): 719.2739, Found: 719.2747.

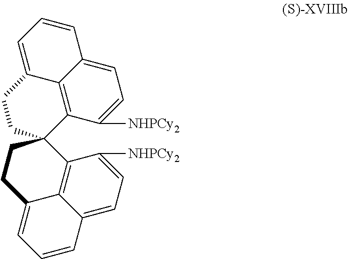

(S)-XVIIIb was prepared as white foam from(S)-Ia (>99% ee, 350.0 mg, 1.0 mmol) and dicyclohexylchlorophosphine (699 mg, 3.0 mmol) according to the General Procedure (eluent: hexanes/DCM=4:1) in 64% yield (475 mg).

¹H NMR (400 MHZ, CDCl₃) δ 7.88-7.75 (m, 2H), 7.70-7.52 (m, 4H), 7.30-7.10 (m, 4H), 3.88 (d, J=10.2 Hz, 2H), 3.44-3.25 (m, 2H), 3.10-2.93 (m, 2H), 2.69-2.56 (m, 2H), 2.56-2.42 (m, 2H), 1.68-1.49 (m, 12H), 1.49-1.23 (m, 9H), 1.15-0.93 (m, 12H), 0.71-0.56 (m, 2H), 0.49-0.24 (m, 4H).

¹³C NMR (101 MHZ, CDCl₃) δ 143.72, 143.58, 132.49, 130.27, 128.45, 127.65 (d, J=2.2 Hz), 126.58, 124.85, 121.68, 118.70, 118.52, 118.24, 39.83, 36.69, 36.55, 35.00, 34.91, 28.51, 28.45, 28.36, 28.16, 27.99, 26.97, 26.90, 26.84, 26.72, 26.67, 26.57, 26.32, 26.10, 25.85, 25.81.

³¹P NMR (162 MHz, CDCl₃) δ 37.9.

HRMS (ES+) Calcd for C₄₉H₆₅N₂P₂⁺ (M+H⁺): 743.4617, Found: 743.4619.

### Synthesis of Chiral Urea XIX

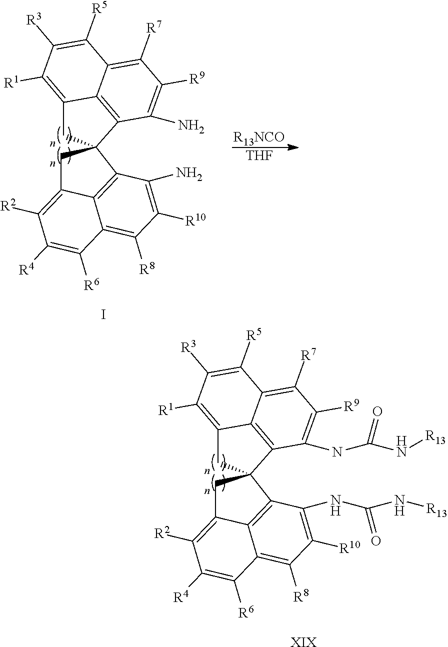

General Procedure: In a dry flask equipped with a stir bar, chiral diamine I (175 mg, 0.5 mmol) was dissolved in dry THF (10.0 mL) at room temperature and R₁₃NCO (1.5 mmol) was added dropwise. Then the mixture was stirred at room temperature. Upon completion (˜12 h), the mixture was concentrated. The residue was subjected to silica gel flash chromatography to give the pure product XIX.

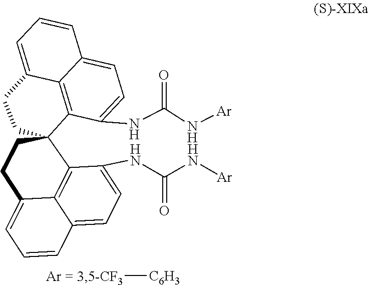

(S)-XIXa was prepared as white foam from(S)-Ia (>99% ee, 175.0 mg, 0.5 mmol) and 3,5-Bis(trifluoromethyl)phenyl isocyanate (765 mg, 1.5 mmol) according to the General Procedure (eluent: hexanes/EA=1:1) in 95% yield (409 mg).

¹H NMR (400 MHZ, CDCl₃) δ 7.48-7.41 (m, 4H), 7.38 (d, J=8.7 Hz, 2H), 7.33-7.25 (m, 6H), 7.23-7.17 (m, 2H), 7.09 (d, J=8.6 Hz, 2H), 6.94 (s, 2H), 5.82 (s, 2H), 3.21-3.02 (m, 2H), 2.99-2.81 (m, 2H), 2.43-2.30 (m, 2H), 2.05-1.93 (m, 2H).

¹³C NMR (101 MHZ, CDCl₃) 152.9, 139.6, 137.2, 134.1, 132.3, 131.7 (q, J=33.1 Hz), 129.6, 129.5, 127.3, 127.1, 126.5, 125.8, 125.0, 123.1 (q, J=271.0 Hz), 118.9, 116.0, 41.4, 32.1, 26.2.

¹⁹F NMR (376 MHZ, Chloroform-d) δ −63.2.

HRMS (ES+) Calcd for C₄₃H₂₈F₁₂N₄NaO₂ (M+Na⁺): 883.1913, Found: 883.1917.

### Synthesis of Chiral Thiourea XX

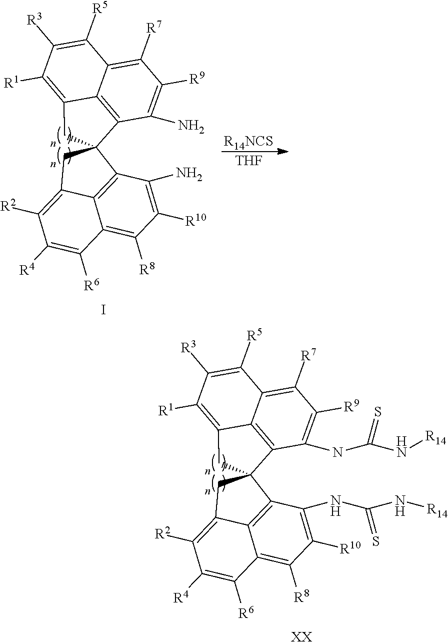

General Procedure: In a dry flask equipped with a stir bar, chiral diamine I (175 mg, 0.5 mmol) was dissolved in dry THF (10.0 mL) at room temperature and R₁₄NCS (1.5 mmol) was added dropwise. Then the mixture was stirred at room temperature. Upon completion (˜12 h), the mixture was concentrated. The residue was subjected to silica gel flash chromatography to give the pure product XX.

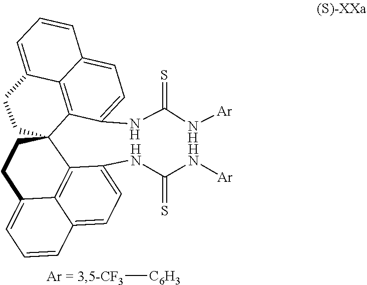

(S)-XXa was prepared as white foam from(S)-Ia (>99% ee, 175.0 mg, 0.5 mmol) and 3,5-Bis(trifuoromethyl)phenyl isothiocyanate (813 mg, 1.5 mmol) according to the General Procedure (eluent: hexanes/EA=1:1) in 95% yield (423 mg).

¹H NMR (400 MHZ, DMSO-d₆) δ 9.47 (s, 2H), 7.94-7.30 (m, 16H), 7.30-7.09 (m, 2H), 3.38-3.22 (m, 2H), 3.17-2.98 (m, 2H), 2.55-2.39 (m, 4H).

¹³C NMR (101 MHZ, DMSO-d₆) δ 179.2, 140.4, 138.5, 135.0, 132.7, 130.8, 130.2 (q, J=32.0 Hz), 129.2, 125.8, 125.7, 125.6, 124.6, 123.2 (q, J=272.0 Hz), 123.1, 117.2, 41.5, 30.7, 26.0.

¹⁹F NMR (376 MHz, Chloroform-d) δ −61.5.

HRMS (ES+) Calcd for C₄₃H₂₈F₁₂N₄NaS₂ (M+Na⁺): 915.1456, Found: 915.1459.

### Synthesis of Chiral Amide XXI

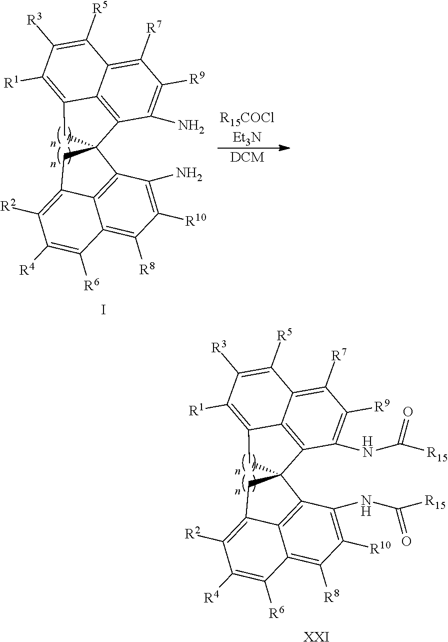

General Procedure: In a dry flask equipped with a stir bar, chiral diamine I (175 mg, 0.5 mmol) was dissolved in dry DCM (10.0 mL) at room temperature. Then Et₃N (505 mg, 5 mmol) and R₁₅COCl (2.0 mmol) was added dropwise. Then the mixture was stirred at room temperature. Upon completion (˜12 h), the mixture was concentrated. The residue was subjected to silica gel flash chromatography to give the pure product XXI.

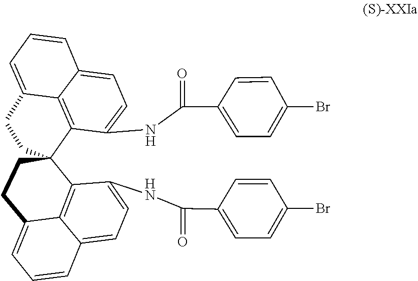

(S)-XXIa was prepared as white foam from (S)-Ia (>99% ee, 175.0 mg, 0.5 mmol) and 4-Bromobenzoyl chloride (440 mg, 2.0 mmol) according to the General Procedure (eluent: hexanes/EA=1:1) in 95% yield (332 mg).

¹H NMR (400 MHZ, Chloroform-d) δ 8.03-7.85 (m, 2H), 7.85-7.69 (m, 4H), 7.52-7.38 (m, 2H), 7.34-7.28 (m, 2H), 7.12 (d, J=8.1 Hz, 4H), 6.54 (d, J=8.1 Hz, 4H), 3.43-3.20 (m, 2H), 3.20-2.95 (m, 2H), 2.67-2.46 (m, 2H), 2.36-2.08 (m, 2H).

¹³C NMR (101 MHz, Chloroform-d) δ 164.4, 134.1, 132.7, 132.0, 131.4, 131.2, 131.2, 129.0, 128.0, 127.8, 126.8, 126.1, 125.8, 125.7, 125.2, 41.7, 31.2, 26.2.

HRMS (ES+) Calcd for C₃₉H₂₈Br₂N₂NaO₂ (M+Na⁺): 737.0410, Found: 737.0414.

### Synthesis of Chiral Diiodine XXII

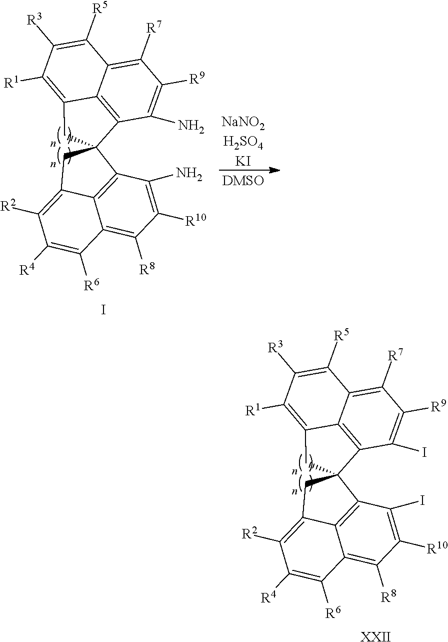

General Procedure: Under water bath, to a solution of chiral diamine I ((>99% ee, 350 mg, 1.0 mmol), NaNO₂ (552 mg, 8.0 mmol), and KI (1.66 g, 10.0 mmol) in DMSO (20 mL) was added aqueous sulfuric acid (2.0 M, 5.0 mL) dropwise. After stirring for 3 h, the reaction mixture was poured into a saturated aqueous solution of NaHCO₃ (50 mL). The resulting mixture was extracted with EtOAc (30 mL×3), and the combined organic layers were washed with Na₂S₂O₃ and brine, dried over Na₂SO₄, and concentrated. The residue was purified by column (eluent: hexanes/DCM=10:1) to afford the desired product XXII as a white solid in 63% yield (400 mg).

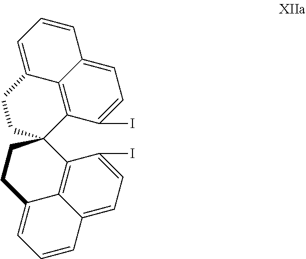

(S)-XXIa was prepared as white foam from(S)-Ia (>99% ee, 350 mg, 1.0 mmol) NaNO₂ (552 mg, 8.0 mmol), KI (1.66 g, 10.0 mmol), and aqueous sulfuric acid (2.0 M, 5.0 mL) in DMSO (20 mL) according to the General Procedure (eluent: hexanes/EA=1:1) in 63% yield (400 mg).

¹H NMR (400 MHZ, CDCl₃) δ 7.88 (d, J=8.6 Hz, 2H), 7.70 (d, J=8.1 Hz, 2H), 7.49-7.42 (m, 2H), 7.41-7.31 (m, 4H), 3.49-3.35 (m, 2H), 3.19-3.09 (m, 2H), 2.91-2.77 (m, 2H), 2.55-2.44 (m, 2H).

¹³C NMR (101 MHz, CDCl₃) δ 142.6, 139.7, 133.8, 133.7, 132.7, 127.9, 126.6, 125.7, 125.2, 93.9, 47.9, 30.4, 26.4.

HRMS (CI) Calcd for C₂₈H₁₈₁₂ (M): 571.9484, Found: 571.9484.

### Synthesis of Phosphate Ligand XXIII

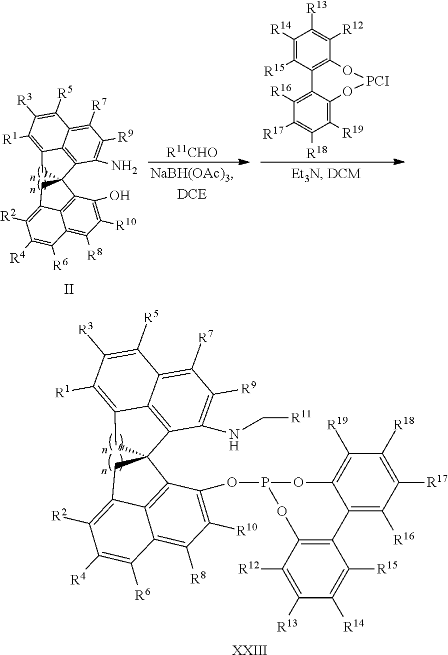

General Procedure: To a solution of II (>99% ee, 0.2 mmol) in anhydrous DCE (2.0 mL) was added sodium triacetoxyborohydride (126.0 mg, 0.6 mmol), and aldehyde (0.3 mmol). The mixture was stirred at room temperature for 12 h before it was directly subjected to silica gel flash chromatography (eluent: hexanes/EtOAc=2:1) to afford the intermediate as a yellow foam.

To a solution of intermediate in anhydrous DCM (2.0 mL) was added triethylamine (90 mmol), and a solution of phosphorochloridite (0.5 mmol). The mixture was stirred at room temperature for 2 h before it was directly subjected to silica gel flash chromatography (eluent: hexanes/EtOAc=2:1) to afford the product XXIIIa.

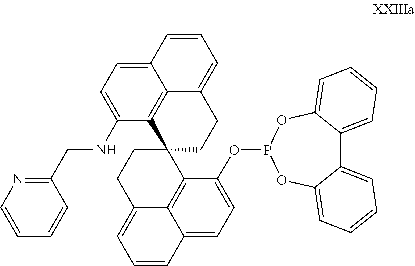 [SMILES: c1ccc(CNc2ccc3cccc4c3c2[C@]2(CC4)CCc3cccc4ccc(Op5oc6ccccc6c6ccccc6o5)c2c34)nc1]

(S)-XXIa was prepared as white foam from(S)-IIa (>99% ee, 70 mg, 0.2 mmol), sodium triacetoxyborohydride (126.0 mg, 0.6 mmol), and 2-pyridinecarboxaldehyde (32.0 mg, 0.3 mmol) in anhydrous DCE (2.0 mL) according to the General Procedure (eluent: hexanes/EA=1:1) in 65% yield (44 mg).

¹H NMR (400 MHZ, CDCl₃) δ 7.98 (d, J=4.9 Hz, 1H), 7.76-7.59 (m, 4H), 7.45-7.27 (m, 8H), 7.25-6.88 (m, 8H), 6.81-6.70 (m, 1H), 5.48 (d, J=8.1 Hz, 1H), 4.32-4.07 (m, 2H), 3.50-3.30 (m, 2H), 3.18-3.04 (m, 2H), 2.69-2.39 (m, 4H).

¹³C NMR (101 MHz, CDCl₃) δ 158.0, 148.5, 140.2, 136.0, 135.2, 133.2, 131.9, 130.8, 130.5, 130.2, 129.8, 129.7, 129.3, 128.9, 128.74, 128.67, 128.2, 127.9, 126.8, 126.5, 125.2, 124.7, 124.54, 124.48, 122.1, 122.0, 121.4, 121.2, 120.8, 115.2, 49.5, 40.7, 30.5, 28.7, 26.8, 26.5.

³¹P NMR (162 MHZ, CDCl₃) δ 142.5.

HRMS (ES+) Calcd for C₄₃H₃₄N₂O₃P⁺ (M+H⁺): 657.2302, Found: 657.2304.

### Synthesis of Thiourea XXIV

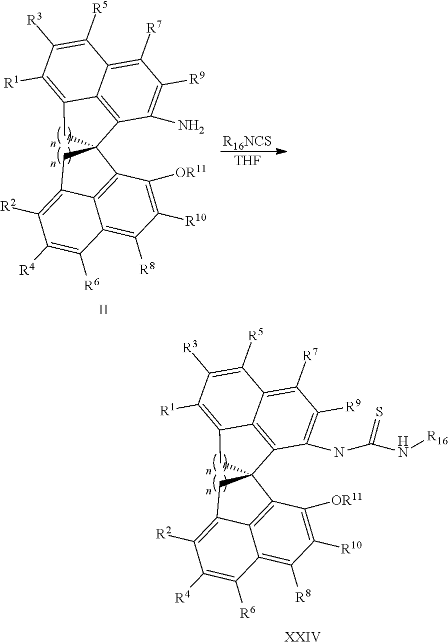

General Procedure A: In a dry flask equipped with a stir bar, chiral amino naphthol II (175 mg, 0.2 mmol) was dissolved in dry THF (10.0 mL) at room temperature and R₁₄NCS (1.5 mmol) was added dropwise. Then the mixture was stirred at room temperature. Upon completion (˜12 h), the mixture was concentrated. The residue was subjected to silica gel flash chromatography to give the pure product XXIV.

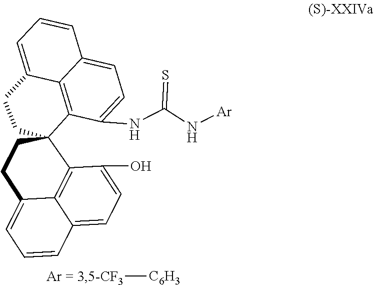

(S)-XXIVa was prepared as white foam from(S)-IIa (>99% ee, 175.0 mg, 0.5 mmol) and 3,5-Bis(trifuoromethyl)phenyl isothiocyanate (407 mg, 0.75 mmol) according to the General Procedure A (eluent: hexanes/EA=1:1) in 92% yield (296 mg).

¹H NMR (400 MHZ, Chloroform-d) δ 7.95-7.65 (m, 2H), 7.64-7.44 (m, 4H), 7.44-7.16 (m, 7H), 6.91 (s, 1H), 6.78 (d, J=8.8 Hz, 1H), 4.51 (s, 1H), 3.44-3.17 (m, 2H), 3.17-2.94 (m, 2H), 2.64-2.26 (m, 3H), 2.18-1.97 (m, 1H).

¹⁹F NMR (376 MHz, Chloroform-d) δ −61.5.

HRMS (ES+) Calcd for C₃₄H₂₆F₆N₂NaOS (M+Na⁺): 647.1562, Found: 647.1563.

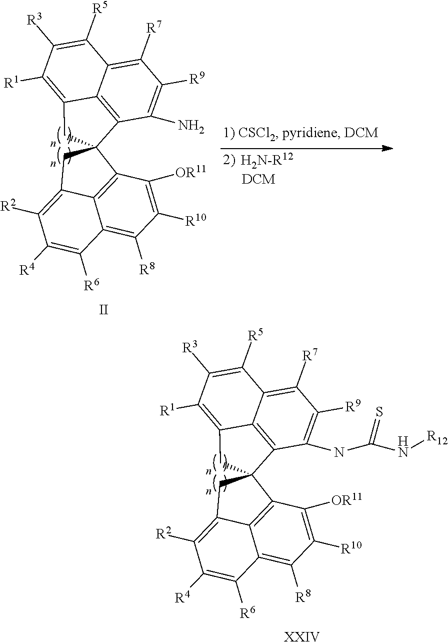

General Procedure B: To a solution of II (0.2 mmol) in anhydrous DCM (2.0 mL) was added pyridine (63.2 mg, 0.8 mmol), and CSCl₂ (27.6 mg, 0.24 mmol). Then the mixture was stirred at room temperature for 2 h. Next, the solvent was evaporated and the residue was purified by column (eluent: hexanes/DCM=2:1) to afford the isothiocyanate as a white solid.

To a solution of the above isothiocyanate in anhydrous DCM (2.0 mL) was added pyridine (63.2 mg, 0.8 mmol) and amine (0.3 mmol). The mixture was stirred at room temperature for 12 h before it was directly subjected to silica gel flash chromatography (eluent: hexanes/EtOAc=2:1) to afford the product.

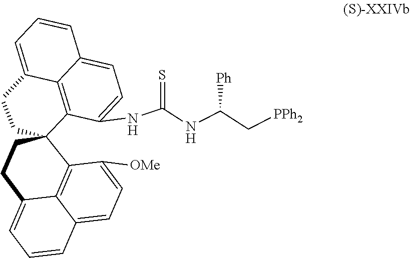

(S)-XXIVb was prepared as white foam from(S)-IIb (>99% ee, 76.0 mg, 0.2 mmol) and (R)-2-(diphenylphosphino)-1-phenylethanamine (91.6 mg, 0.3 mmol) according to the General Procedure B (eluent: hexanes/EA=1:1) in 70% yield (100 mg).

¹H NMR (400 MHZ, CDCl₃) δ 7.88-7.80 (m, 1H), 7.78-7.66 (m, 3H), 7.50-7.27 (m, 14H), 7.24-7.14 (m, 5H), 7.04-6.90 (m, 1H), 6.71 (s, 1H), 5.72 (s, 1H), 3.38-3.21 (m, 2H), 3.18 (s, 3H), 3.13-3.04 (m, 1H), 2.99-2.87 (m, 2H), 2.78-2.46 (m, 2H), 2.45-2.27 (m, 2H), 2.07-1.93 (m, 2H).

¹³C NMR (101 MHZ, CDCl₃) δ 179.2, 152.1, 141.1, 140.0, 138.3 (d, J=13.1 Hz), 137.6 (d, J=12.3 Hz), 135.5, 133.8, 130.5, 130.0, 129.6, 128.9, 128.60, 128.55, 128.43, 128.38, 128.3, 127.5, 126.7, 126.6, 126.5, 126.4, 126.3, 125.9, 125.4, 125.2, 124.7, 124.0, 114.1, 56.4, 56.2, 55.9, 41.2, 36.2 (d, J=15.6 Hz), 30.4 (d, J=13.8 Hz), 26.5 (d, J=23.6 Hz).

³¹P NMR (162 MHZ, CDCl₃) δ −24.3.

HRMS (ES−) Calcd for C₄₇H₄₂OPS⁻ (M−H⁺): 711.2604, Found: 711.2599.

### Synthesis of Phosphoramidite XXV

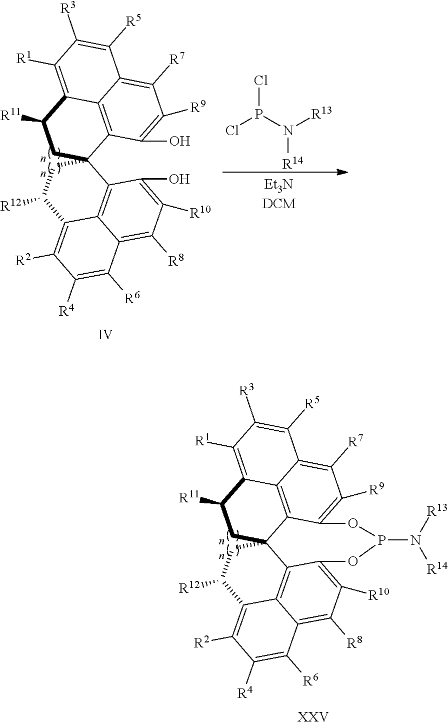

General Procedure: At 0° C. under N₂, to a solution of IV (99% ee, 1.0 mmol) and Et₃N (1.4 mL, 10 mmol) in anhydrous THF (5.0 mL) was added phosphoramidous dichloride (3.0 mmol). Next, the mixture was stirred at room temperature. Upon completion, the mixture was diluted with Et₂O (20 mL), washed with H₂O and brine, dried over Na₂SO₄, and concentrated. The residue was purified by silica gel flash column chromatography (eluent: n-hexane/EtOAc=20:1) to afford the desired product XXV.

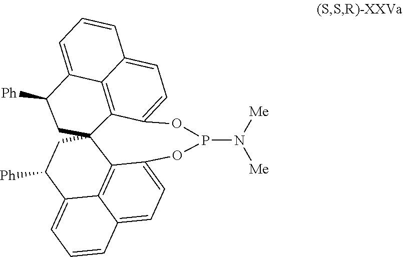

(S,S,R)-XXVa was prepared as white foam from (S,S,R)-IVa (>99% ee, 504 mg, 1.0 mmol), Et₃N (1.4 mL, 10 mmol), and dimethylphosphoramidous dichloride (435 mg, 3.0 mmol). according to the General Procedure (eluent: hexanes/EA=20:1) in 80% yield (462 mg).

¹H NMR (400 MHZ, CDCl₃) δ 7.77-7.61 (m, 4H), 7.31 (d, J=8.6 Hz, 1H), 7.26-7.08 (m, 8H), 7.07-6.95 (m, 5H), 6.85 (d, J=7.1 Hz, 1H), 6.80 (d, J=7.1 Hz, 1H), 4.23 (d, J=5.2 Hz, 2H), 2.90 (ddd, J=13.9, 8.7, 5.6 Hz, 2H), 2.18-2.00 (m, 8H).

¹³C NMR (101 MHZ, CDCl₃) δ 146.3 (d, J=4.9 Hz), 144.6, 144.2, 142.9 (d, J=4.8 Hz), 136.4, 136.2, 134.0 (d, J=6.1 Hz), 131.9 (d, J=2.1 Hz), 130.8 (d, J=2.1 Hz), 130.3, 130.2 (d, J=2.4 Hz), 129.9, 128.1, 128.0, 127.2, 127.1 (d, J=4.2 Hz), 126.7, 125.2, 125.1 (d, J=1.9 Hz), 124.9, 124.7, 123.9, 123.1 (d, J=4.2 Hz), 122.6 (s), 122.5 (d, J=4.3 Hz), 48.3, 47.4, 44.4, 41.8, 41.8, 34.4, 34.2.

³¹P NMR (162 MHZ, CDCl₃) δ 120.15.

HRMS (ES+) calcd for C₃₉H₃₃NO₂P [M+H]⁺: 578.2249, Found: 578.2247.

### Synthesis of Chiral Biphosphine XXVI

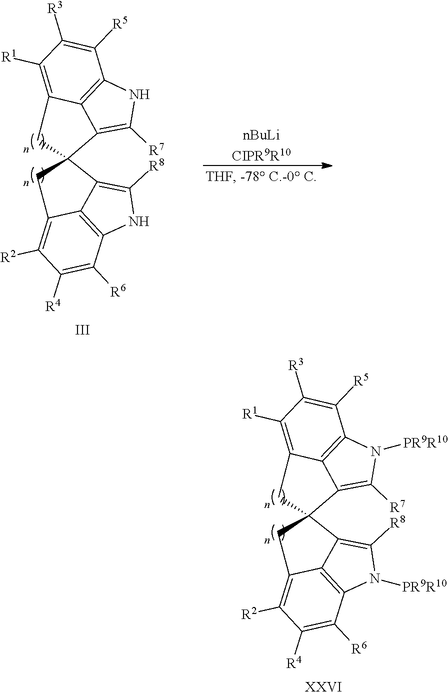

General Procedure: In a dry flask equipped with a stir bar, chiral diindole III (0.2 mmol) was dissolved in dry THF (2.0 mL) at −78° C. and ⁿBuLi (2.4 M in hexanes, 33.3 μL, 0.8 mmol) was added dropwise. After that the reaction solution was stirred at 0° C. for 1 h. Then ClPR⁹R¹⁰ (0.8 mmol) was added at 0° C. Then the mixture was allowed to warm to room temperature for 5 h. Upon completion, the mixture was quenched by NH₄Cl aq, extracted with Et₂O (20 mL×3), washed with H₂O and brine, dried over Na₂SO₄, and concentrated. The residue was subjected to silica gel flash chromatography to give the pure product XXVI.

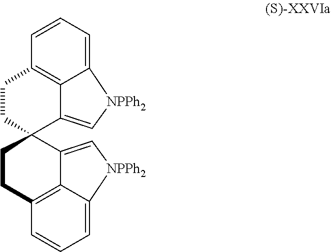

(S)-XXVIa was prepared as white foam from(S)-IIIa (>99% ee, 60 mg, 0.2 mmol) and chlorodiphenylphosphine (143.6 μL, 0.8 mmol) according to the General Procedure (eluent: hexanes/DCM=10:1) in 61% yield (81 mg).

¹H NMR (400 MHZ, CDCl₃) δ 7.52 (d, J=8.1 Hz, 2H), 7.46-7.27 (m, 19H), 7.25-7.18 (m, 3H), 7.02 (d, J=7.1 Hz, 2H), 6.66 (d, J=2.0 Hz, 2H), 3.25-3.13 (m, 2H), 3.06-2.93 (m, 2H), 2.28-2.13 (m, 4H).

¹³C NMR (101 MHZ, CDCl₃) δ 139.7, 139.5, 136.9, 136.74, 136.66, 136.5, 132.1, 131.9, 131.8, 131.6, 131.4, 129.5, 129.3, 128.9, 128.8, 128.53, 128.50, 128.47, 128.4, 125.5, 123.5, 122.7, 117.4, 109.7, 109.6, 36.0, 35.3, 24.5.

³¹P NMR (162 MHZ, CDCl₃) δ 36.76.

HRMS (ES+) Calcd for C₄₅H₃₆N₂P₂ (M+H⁺): 667.2426, Found: 667.2418.

### Synthesis of Chiral Biphosphine XXVII

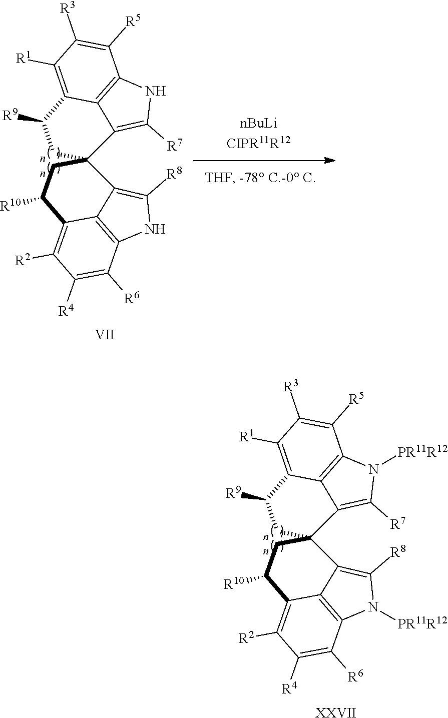

General Procedure: In a dry flask equipped with a stir bar, chiral diaryl diindole VII (1.0 mmol) was dissolved in dry THF (10 mL) at −78° C. and ⁿBuLi (2.4 M in hexanes, 1.67 mL, 4.0 mmol) was added dropwise. After that the reaction solution was stirred at 0° C. for 1 h. Then ClPR₁₁R₁₂ (4.0 mmol) was added at 0° C. Then the mixture was allowed to warm to room temperature for 5 h. Upon completion, the mixture was quenched by NH₄Cl aq, extracted with Et₂O (20 mL×3), washed with H₂O and brine, dried over Na₂SO₄, and concentrated. The residue was subjected to silica gel flash chromatography to give the pure product XXVII.

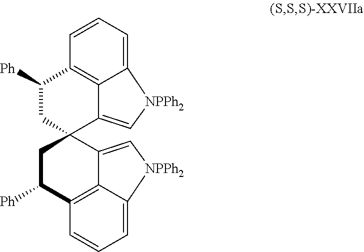

(S,S,S)-XXVIIa was prepared as white foam from (S,S)-VIIa (>99% ee, 450 mg, 1.0 mmol) and chlorodiphenylphosphine (718 μL, 4.0 mmol) according to the General Procedure (eluent: hexanes/DCM=10:1) in 83% yield (683 mg).

¹H NMR (400 MHZ, CDCl₃) δ 7.45-7.31 (m, 14H), 7.25-7.13 (m, 14H), 7.10-7.01 (m, 6H), 6.81 (d, J=2.5 Hz, 2H), 6.64 (d, J=7.3 Hz, 2H), 4.48-4.37 (m, 2H), 2.35-2.27 (m, 2H), 2.25-2.15 (m, 2H).

¹³C NMR (101 MHz, CDCl₃) δ 144.3, 139.2, 139.1, 136.6, 136.54, 136.47, 136.4, 134.0, 132.4, 132.2, 131.7, 131.5, 129.6, 129.4, 128.65, 128.60, 128.5, 128.2, 126.2, 125.5, 123.8, 122.6, 118.0, 110.1, 110.0, 47.3, 43.1, 36.6.

³¹P NMR (162 MHZ, CDCl₃) δ 37.65.

HRMS (ES+) Calcd for C₅₇H₄₄N₂P₂ (M+Na⁺): 841.2872, Found: 841.2876.

### Synthesis of Chiral Biphosphine XXXXXXI

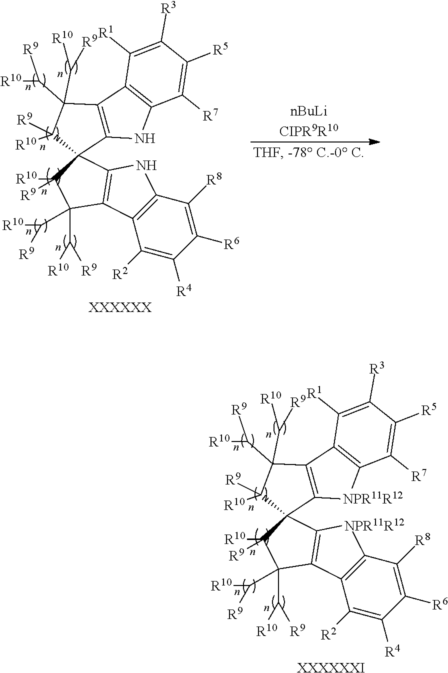

General Procedure: In a dry flask equipped with a stir bar, chiral diindole XXXXXX (0.2 mmol) was dissolved in dry THF (2.0 mL) at −78° C. and ⁿBuLi (2.4 M in hexanes, 33.3 μL, 0.8 mmol) was added dropwise. After that the reaction solution was stirred at 0° C. for 1 h. Then ClPR⁹R¹⁰ (0.8 mmol) was added at 0° C. Then the mixture was allowed to warm to room temperature for 5 h. Upon completion, the mixture was quenched by NH₄Cl aq, extracted with Et₂O (20 mL×3), washed with H₂O and brine, dried over Na₂SO₄, and concentrated. The residue was subjected to silica gel flash chromatography to give the pure product XXXXXXI.

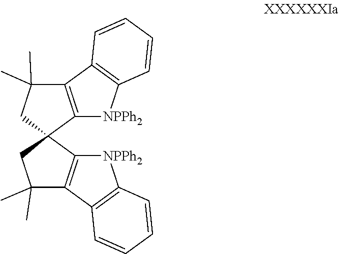

(S)-XXXXXXIa was prepared as white foam from(S)—XXXXXXI (>99% ee, 70.8 mg, 0.2 mmol) and chlorodiphenylphosphine (143.6 μL, 0.8 mmol) according to the General Procedure (eluent: hexanes/DCM=10:1) in 82% yield (126.6 mg).

¹H NMR (400 MHZ, CDCl₃) δ 7.64 (d, J=7.7 Hz, 2H), 7.59-7.51 (m, 4H), 7.46-7.39 (m, 6H), 7.18-7.09 (m, 8H), 7.04-6.97 (m, 4H), 6.83-6.76 (m, 2H), 6.63 (d, J=8.4 Hz, 2H), 2.93 (d, J=13.1 Hz, 2H), 2.58 (d, J=13.1 Hz, 2H), 1.55 (s, 6H), 1.48 (s, 6H).

¹³C NMR (101 MHz, CDCl₃) δ 149.7, 149.5, 144.2, 144.1, 136.2, 136.0, 135.0, 134.8, 131.6, 131.4, 130.94, 130.91, 130.88, 130.5, 130.3, 129.7, 129.1, 128.9, 128.54, 128.51, 128.47, 128.0, 127.94, 127.89, 127.0, 120.4, 120.2, 118.4, 116.0, 61.4, 52.33, 52.29, 52.2, 38.2, 30.2, 29.3.

³¹P NMR (162 MHZ, CDCl₃) δ 35.14.

HRMS (ES+) Calcd for C₄₉H₄₄N₂P₂ (M+H⁺): 723.3053, Found: 723.3064.

### Synthesis of Chiral Phosphoramidite XXXXXXVII

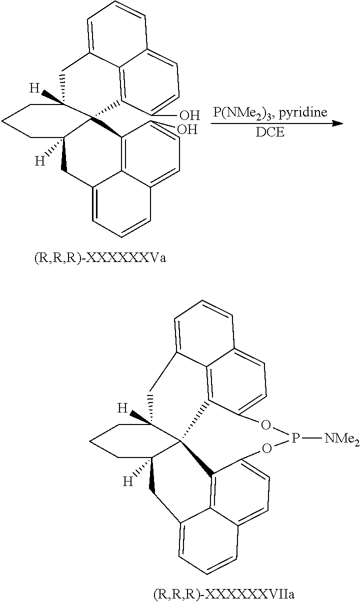

General Procedure: In a dry flask equipped with a stir bar, chiral dios (R,R,R)-XXXXXXVa (0.2 mmol) and pyridine (0.8 mmol) was dissolved in dry DCE (2.0 mL) and P(NMe₂)₃ (0.24 mmol) was added dropwise. After that the reaction solution was stirred at room temperature for 2 h. Upon completion, the mixture was concentrated. The residue was subjected to silica gel flash chromatography to give the pure product (R,R,R)-XXXXXXVIIa in 70% yield.

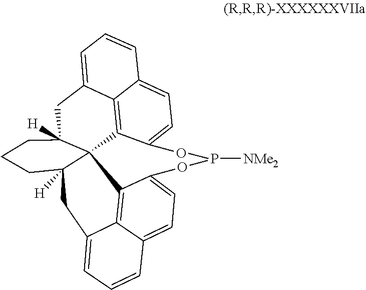

¹H NMR (400 MHZ, CDCl₃) δ 7.73-7.60 (m, 4H), 7.38-7.28 (m, 2H), 7.17 (m, 3H), 6.95 (d, J=8.6 Hz, 1H), 3.23-2.60 (m, 6H), 2.15 (d, J=9.2 Hz, 6H), 1.39-1.14 (m, 6H).

¹³C NMR (101 MHZ, CDCl₃) δ 134.6, 134.1, 132.1, 130.9, 130.6, 127.8, 127.3, 125.0, 124.9, 124.4, 124.3, 123.8, 123.7, 123.34, 123.29, 46.8, 37.4, 36.2, 35.8, 35.6, 32.7, 32.3, 25.1, 25.0, 17.0.

³¹P NMR (162 MHZ, CDCl₃) δ 125.00.

HRMS (ES+) Calcd for C₄₅H₃₆N₂P₂ (M+H⁺): 667.2426, Found: 667.2418.

### Application of Chiral Ligands and Catalysts

### Application of (S)-XVIIIa

### Asymmetric Hydrogenation of Dehydroamino Acid Derivatives XXVIIIa

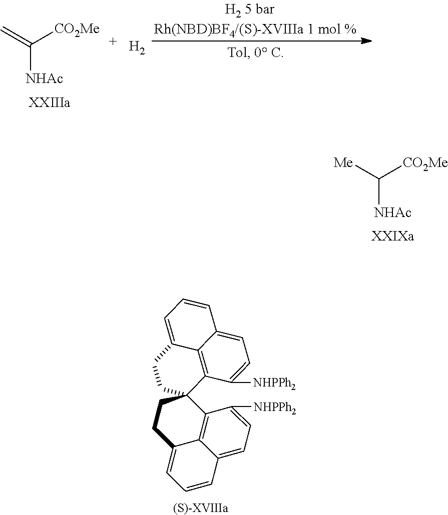

To a vial with a magnetic stirring bar were added Rh(NBD)₂BF₄ (7.4 mg, 0.02 mmol), (S)-VIIIa (14.4 mg, 0.02 mmol), and anhydrous DCM (2.0 mL) under nitrogen. The mixture was stirred 30 min. Then solution of chiral Rhodium complex (0.1 mL) was added to dehydroamino acid derivatives XIIIa (29 mg, 0.2 mmol) in anhydrous Tol (2.0 mL). The reaction mixture was transferred into the autoclave, which was back-and-filled with H2 gas (<5 bar) for three times and finally, the autoclave was pressured under 5 bar H₂. The mixture was stirred at room temperature for 12 hours. The solution was concentrated in vacuo, and an aliquot was sampled for determination of the conversion by ¹H NMR. The residue was purified by silica gel column chromatography to afford the hydrogenated product XIVa as a colorless oil in 98% yield (28.3 mg, 92% ee).

HPLC analysis of the product: Daicel CHIRALCEL OD-H column; 5% i-PrOH in hexanes; 1.0 mL/min; retention times: 12.5 min (major), 15.8 min (minor).

¹H NMR (400 MHZ, CDCl₃) δ 6.16 (s, 1H), 4.72-4.47 (m, 1H), 4.19 (q, J=7.1 Hz, 2H), 2.00 (s, 3H), 1.38 (d, J=7.2 Hz, 3H), 1.27 (t, J=7.2 Hz, 3H).

¹³C NMR (101 MHZ, CDCl₃) δ 173.2, 169.5, 61.5, 48.1, 23.1, 18.6, 14.1.

### Asymmetric Hydrogenation of Intermediate of Sitagliptin XXXa

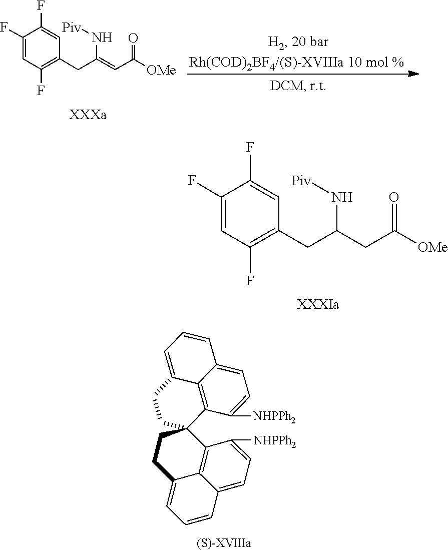

To a vial with a magnetic stirring bar were added Rh(COD)₂BF₄ (4.1 mg, 0.01 mmol), (S)-XVIIIa (8.6 mg, 0.012 mmol), and anhydrous DCM (1.0 mL) under nitrogen. The mixture was stirred 30 min. Then to the solution of chiral Rhodium complex was added dehydroamino acid derivatives XXXa (33 mg, 0.1 mmol). The reaction mixture was transferred into the autoclave, which was back-and-filled with H₂ gas (<5 bar) for three times and finally, the autoclave was pressured under 20 bar H₂. The mixture was stirred at room temperature for 12 hours. The solution was concentrated in vacuo, and an aliquot was sampled for determination of the conversion by ¹H NMR. The residue was purified by silica gel column chromatography to afford the hydrogenated product XXXIa as a colorless oil in 95% yield (31.3 mg, 80% ee).

HPLC analysis of the product: Daicel CHIRALCEL OD-H column; 2% i-PrOH in hexanes; 1.0 mL/min; retention times: 15.5 min (minor), 16.7 min (major).

¹H NMR (400 MHZ, CDCl₃) δ 7.08-6.93 (m, 1H), 6.93-6.78 (m, 1H), 4.46-4.25 (m, 1H), 3.66 (s, 3H), 2.89-2.73 (m, 2H), 2.63-2.43 (m, 2H), 1.08 (s, 9H).

¹³C NMR (101 MHZ, CDCl₃) δ 177.9, 172.1, 121.3, 121.1, 119.0 (d, J=6.1 Hz), 118.8 (d, J=5.6 Hz), 105.3 (d, J=20.8 Hz), 105.0 (d, J=20.7 Hz), 51.7, 46.2, 38.5, 37.1, 32.4, 27.2.

HRMS (ES+) Calcd for C₁₆H₂₀F₃NNaO₃ (M+Na⁺): 354.1287, Found: 354.1291.

### Application of (S)-XXIIIa

Asymmetric Allylic Substitution of (E)-1,3-diphenylallyl acetate

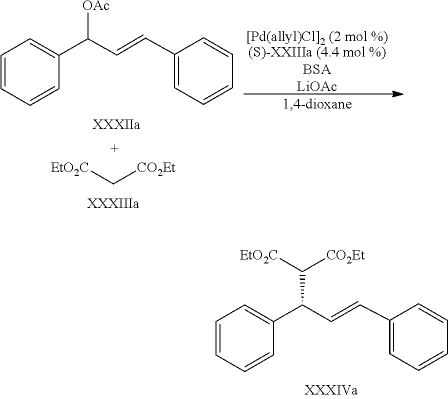 [SMILES: CC(=O)OC(/C=C/c1ccccc1)c1ccccc1 ; "(S)-XXIIIa (4.4 mol %)" ; CCOC(=O)CC(=O)OCC ; CCOC(=O)C(C(=O)OCC)[C@H](/C=C/c1ccccc1)c1ccccc1]

Under N₂, to a vial with a magnetic stirring bar were added [Pd(allyl)Cl]₂ (1.6 mg, 0.005 mmol), (S)-XXIIIa (7.9 mg, 0.012 mmol), and anhydrous DCM (0.5 mL) under nitrogen. The mixture was stirred for 1 h to form a solution of chiral palladium complex. Then to a solution of (E)-1,3-diphenylallyl acetate XXXIIa (25.2 mg, 0.1 mmol), dimethyl malonate XXXIIIa (0.2 mmol), LiOAc (19.2 mg, 0.3 mmol) and BSA (60.9 mg, 0.3 mmol) in anhydrous 1,4-dioxane (1.0 mL) was added the above solution of chiral palladium complex (0.2 mL). The mixture was stirred at room temperature for 12 hours. Upon completion, directly subjected to silica gel flash chromatography (eluent: hexanes/DCM=2:1) to afford the product XXXIVa as a colorless oil in 93% yield (32.1 mg, 91% ee).

[α]_{D}²⁰: −13.3 (c=1.0, CH₂Cl₂). HPLC analysis of the product: Daicel CHIRALPAK AD-H column; 5% i-PrOH in hexanes; 1.0 ml/min; retention times: 16.8 min (minor), 20.1 min (major).

¹H NMR (400 MHZ, CDCl₃) δ 7.37-7.15 (m, 10H), 6.49 (d, J=15.7 Hz, 1H), 6.34 (dd, J=15.8, 8.6 Hz, 1H), 4.28 (dd, J=10.9, 8.6 Hz, 1H), 3.97 (d, J=10.9 Hz, 1H), 3.71 (s, 3H), 3.53 (s, 3H).

¹³C NMR (101 MHZ, CDCl₃) δ 168.2, 167.7, 140.1, 136.8, 131.8, 129.1, 128.7, 128.4, 127.8, 127.5, 127.1, 126.3, 57.6, 52.6, 52.4, 49.2.

### Application of (S)-XXIVb

### Asymmetric MBH Reaction

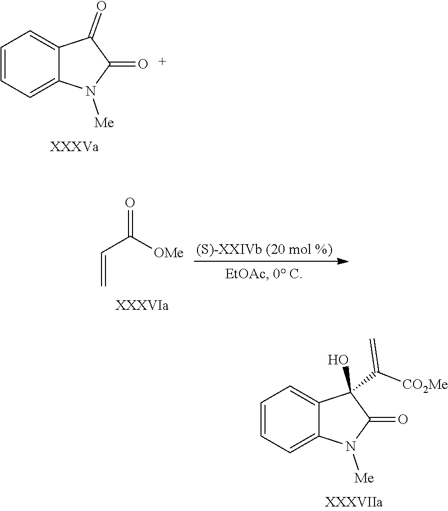

At 0° C., the solution of methyl acrylate XXXVIa (42 μL, 0.45 mmol) and chiral organocatalyst(S)-XXIVb (21.3 mg, 0.03 mmol) in EtOAc (0.5 mL) was added N-methyl isatin XXXVa (24.1 mg, 0.15 mmol). The mixture was stirred at 0° C. for 14 days before it was warmed to room temperature, evaporated and subjected to silica gel flash chromatography (eluent: hexanes/EtOAc=4:1) to afford the product XXXVIIa as a white solid in 86% yield (31.8 mg, 82% ee).

HPLC analysis of the product: Daicel CHIRALCEL OD-H column; 10% i-PrOH in hexanes; 1.0 mL/min; retention times: 11.6 min (minor), 13.2 min (major).

¹H NMR (400 MHZ, CDCl₃) δ 7.36-7.29 (m, 1H), 7.17 (d, J=7.2 Hz, 1H), 7.07-6.99 (m, 1H), 6.86 (d, J=7.8 Hz, 1H), 6.55 (s, 1H), 6.43 (s, 1H), 4.01 (s, 1H), 3.62 (s, 3H), 3.24 (s, 3H).

¹³C NMR (101 MHZ, CDCl₃) δ 176.3, 165.1, 144.5, 139.1, 130.2, 129.3, 127.8, 123.8, 123.0, 108.6, 76.1, 52.0, 26.4.

### Application of (S,S,R)-XXVa

### Asymmetric Allylic Reaction

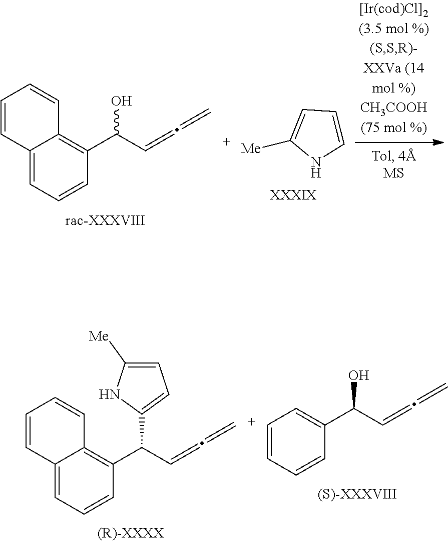

In a glove-box, an oven-dried 100 mL round-bottom flask containing a magnetic stir-bar was charged with rac-XXXVIIIa (1 mmol, 1 equiv), [Ir(cod)Cl]₂ (23.5 mg, 3.5 mol %) and (S,S,R)-XXVa (80.8 mg, 14 mol %). The solids were taken up in toluene (40.0 mL, [rac-XXXVIIIa]=0.025 M) and the resulting solution was stirred under dry Ar gas for 30 minutes. 200 mg 4 Å MS, pyrrole XXXIXa (0.5 mmol, 0.5 eq.) and CH₃COOH (47.2 μL, 75 mol %) were then added in sequence and the mixture was stirred at room temperature. The reaction was monitored by TLC. Upon completion, the mixture was diluted with Et₂O (20 mL), washed with saturated NaHCO₃ aqueous solution and brine, dried over Na₂SO₄, and concentrated. The residual was purified by silica gel flash column chromatography (eluent: n-hexane/EtOAc=20:1→10:1) to give the desired product (R)-XXXXa and (S)-XXXVIIIa.

$$s = ln [ ( 1 - conv . ) ( 1 - {ee}^{( S ) - 5} ) ] / ln [ ( 1 - conv . ) ( 1 + {ee}^{( S ) - 5} ) ] ;$$

$$conv . = {ee}^{( S ) - 5} / ( {ee}^{( S ) - 5} + {ee}^{7} ) .$$

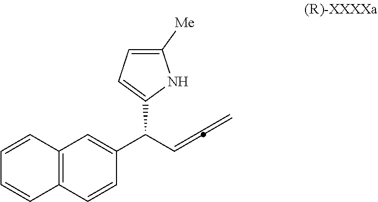

(R)-XXXXa was prepared as a yellow oil from rac-XXXVIIIa (196.1 mg, 1.0 mmol) and 2-methylpyrrole XXXIXa (42 μL, 0.5 mmol) according to the General Procedure (eluent: n-hexane/EtOAc=20:1) in 38% yield (98.2 mg, 90% ee).

HPLC analysis of the product: Daicel CHIRALPAK® AS-H column; 1% i-PrOH in n-hexane; 0.5 mL/min; retention times: 23.7 min (minor), 26.5 min (major).

¹H NMR (400 MHZ, CDCl₃) δ 7.88-7.80 (m, 3H), 7.74 (s, 1H), 7.60 (brs, 1H), 7.54-7.46 (m, 2H), 7.41 (dd, J=8.4, 2.4 Hz, 1H), 5.93 (t, J=2.8 Hz, 1H), 5.86 (t, J=2.4 Hz, 1H), 5.70 (dt, J=8.0, 6.4 Hz, 1H), 4.90-4.78 (m, 3H), 2.23 (s, 3H).

¹³C NMR (101 MHZ, CDCl₃) δ 208.6, 139.9, 133.4, 132.5, 131.7, 128.2, 127.7, 127.6, 127.3, 126.6, 126.3, 126.0, 125.7, 106.6, 105.8, 92.9, 76.7, 44.8, 13.0.

HRMS (ES−) Calcd for C₁₉H₁₆N [M−H]⁻: 258.1288, found: 258.1270.

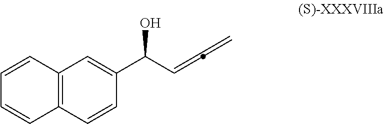

(S)-XXXVIIIa was prepared as a yellow oil from rac-XXXVIIIa (196.1 mg, 1.0 mmol) and 2-methylpyrrole XXXIXa (42 μL, 0.5 mmol) according to the General Procedure (eluent: n-hexane/EtOAc=10:1) in 41% yield (80.4 mg, 95% ee); s=74.

HPLC analysis of the product: Daicel CHIRALPAK® IC column; 5% i-PrOH in n-hexane; 1.0 mL/min; retention times: 11.7 min (major), 13.2 min (minor).

### Application of (S,S,S)-XXVIIa

Asymmetric Allylic Substitution of (E)-1,3-diphenylallyl acetate

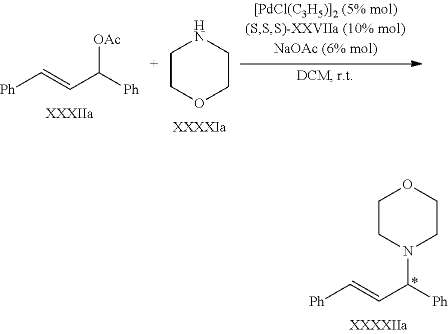

A mixture of [Pd(C₃H₅)Cl)]₂ (1.8 mg, 0.0025 mmol) and (S,S,S)-XXVIIa (4.1 mg, 0.005 mmol) in anhydrous DCM (0.5 mL) was stirred for 1 h under argon atmosphere to form the active catalyst species. The allylic acetate XXXIIa (25.2 mg, 0.1 mmol, 1.0 equiv.) and morpholine XXXXIa (13.1 μL, 0.15 mmol) were added to the reaction mixture, and optionally BSA (37 μL, 0.15 mmol) and NaOAc (2.5 mg, 0.03 mmol) were added. The mixture was stirred at room temperature. Upon completion (˜24 h), the reaction mixture was concentrated in vacuo. and the crude product was purified by silica gel flash chromatography (eluent: hexanes/EtOAc=10:1) to afford XXXXIIa as a colorless oil in 85% yield (23.7 mg, 90% ee).

[α]_{D}²⁵: −10.4 (c=1.8, Acetone). HPLC analysis of the product: Daicel CHIRALPAK® IE-3 column; 5% i-PrOH in hexanes; 1.0 mL/min; retention times: 5.8 min (minor), 6.4 min (major).

¹H NMR (400 MHZ, CDCl₃) δ 7.40 (d, J=7.2 Hz, 2H), 7.37-7.27 (m, 6H), 7.25-7.18 (m, 2H), 6.57 (d, J=15.8 Hz, 1H), 6.33-6.22 (m, 1H), 3.79 (d, J=8.9 Hz, 1H), 3.71 (t, J=4.6 Hz, 4H), 2.70-2.46 (m, 2H), 2.45-2.33 (m, 2H).

¹³C NMR (101 MHz, CDCl₃) δ 141.5, 136.7, 131.6, 131.4, 128.6, 128.5, 128.0, 127.6, 127.3, 126.4, 74.8, 67.1, 52.2.

### Application of (S)-XXXXXXIa

Asymmetric Allylic Amination of ethyl 2-(acetoxy(phenyl)methyl)acrylate

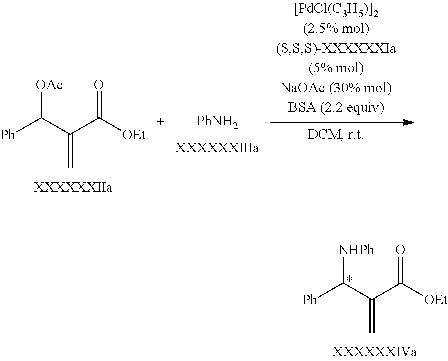

A mixture of [Pd(C₃H₅)Cl)]₂ (0.9 mg, 0.0025 mmol) and (S,S,S)-XXXXXXIa (3.6 mg, 0.005 mmol) in anhydrous DCM (1.0 mL) was stirred for 1 h under argon atmosphere to form the active catalyst species. The ethyl 2-(acetoxy(phenyl)methyl)acrylate XXXXXXIIa (24.8 mg, 0.1 mmol, 1.0 equiv.) and aniline XXXXXXIIIa (30.0 μL, 0.3 mmol) were added to the reaction mixture, and optionally BSA (37 μL, 0.22 mmol) and NaOAc (2.5 mg, 0.03 mmol) were added. The mixture was stirred at room temperature. Upon completion (˜24 h), the reaction mixture was concentrated in vacuo. and the crude product was purified by silica gel flash chromatography (eluent: hexanes/EtOAc=10:1) to afford XXXXXXIVa as a colorless oil in 60% yield (16.9 mg, 90% ee, b:1=2.5:1).

HPLC analysis of the product: Daicel CHIRALPAK® AD-3 column; 5% i-PrOH in hexanes; 1.0 mL/min; retention times: 7.0 min (minor), 7.8 min (major).

¹H NMR (400 MHZ, CDCl₃) δ 7.40-7.26 (m, 5H), 7.20-7.14 (m, 2H), 6.73 (t, J=7.2 Hz, 1H), 6.59 (d, J=8.0 Hz, 2H), 6.40 (s, 1H), 5.95 (s, 1H), 5.42 (s, 1H), 4.22-4.08 (m, 2H), 1.21 (t, J=7.1 Hz, 3H),

¹³C NMR (101 MHZ, CDCl₃) δ 166.2, 146.7, 140.7, 140.2, 130.7, 129.1, 128.7, 127.7, 127.5, 125.9, 117.8, 113.4, 60.8, 59.0, 14.0.

### Asymmetric Hydrogenation of Dehydroamino Acid Derivatives XXXXXXVIIa

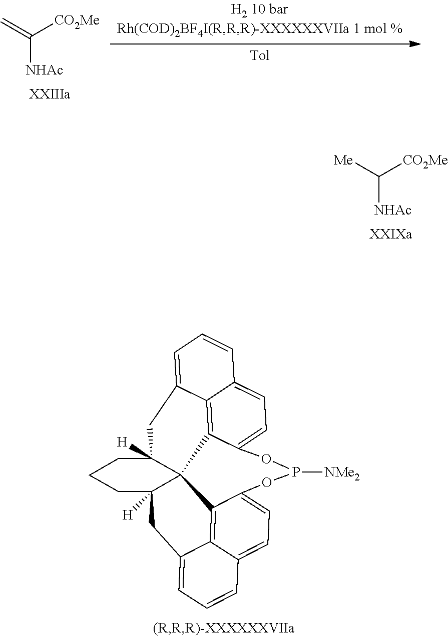

To a vial with a magnetic stirring bar were added Rh(COD)₂BF₄ (4.0 mg, 0.01 mmol), (R,R,R)-XXXXXXVIIa (10.2 mg, 0.022 mmol), and anhydrous DCM (2.0 mL) under nitrogen. The mixture was stirred 30 min. Then solution of chiral Rhodium complex (0.1 mL) was added to dehydroamino acid derivatives XXIIIa (29 mg, 0.2 mmol) in anhydrous Tol (2.0 mL). The reaction mixture was transferred into the autoclave, which was back-and-filled with H₂ gas (<5 bar) for three times and finally, the autoclave was pressured under 10 bar H₂. The mixture was stirred at room temperature for 12 hours. The solution was concentrated in vacuo, and an aliquot was sampled for determination of the conversion by ¹H NMR. The residue was purified by silica gel column chromatography to afford the hydrogenated product XXIXa as a colorless oil in 98% yield (28.4 mg, 96% ee).

HPLC analysis of the product: Daicel CHIRALCEL OD-H column; 5% i-PrOH in hexanes; 1.0 mL/min; retention times: 15.1 min (major), 18.6 min (minor).

¹H NMR (400 MHZ, CDCl₃) δ 6.16 (s, 1H), 4.72-4.47 (m, 1H), 4.19 (q, J=7.1 Hz, 2H), 2.00 (s, 3H), 1.38 (d, J=7.2 Hz, 3H), 1.27 (t, J=7.2 Hz, 3H).

¹³C NMR (101 MHZ, CDCl₃) δ 173.2, 169.5, 61.5, 48.1, 23.1, 18.6, 14.1.

### Asymmetric Hydrogenation of Dehydroamino Acid Derivatives XXXXXXVIIa

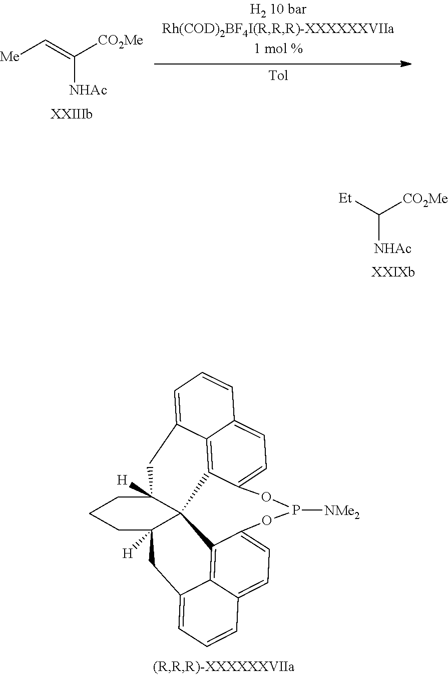

To a vial with a magnetic stirring bar were added Rh(COD)₂BF₄ (4.0 mg, 0.01 mmol), (R,R,R)-XXXXXXVIIa (10.2 mg, 0.022 mmol), and anhydrous DCM (2.0 mL) under nitrogen. The mixture was stirred 30 min. Then solution of chiral Rhodium complex (0.1 mL) was added to dehydroamino acid derivatives XXIIIb (29 mg, 0.2 mmol) in anhydrous Tol (2.0 mL). The reaction mixture was transferred into the autoclave, which was back-and-filled with H₂ gas (<5 bar) for three times and finally, the autoclave was pressured under 10 bar H₂. The mixture was stirred at room temperature for 12 hours. The solution was concentrated in vacuo, and an aliquot was sampled for determination of the conversion by ¹H NMR. The residue was purified by silica gel column chromatography to afford the hydrogenated product XXIXb as a colorless oil in 98% yield (28.4 mg, 92% ee).

HPLC analysis of the product: Daicel CHIRALCEL OD-H column; 5% i-PrOH in hexanes; 1.0 mL/min; retention times: 13.8 min (major), 20.6 min (minor).

¹H NMR δ 6.16 (s, 1H), 4.61-4.51 (m, 1H), 3.72 (s, 3H), 2.01 (s, 3H), 1.92-1.79 (m, 1H), 1.77-1.62 (m, 1H), 0.88 (t, J=7.5 Hz, 3H).

¹³C NMR (101 MHz, CDCl₃) δ 173.0, 169.8, 53.2, 52.3, 25.6, 23.1, 9.4.

### Asymmetric Hydrogenation of Dehydroamino Acid Derivatives XXXXXXVIIa

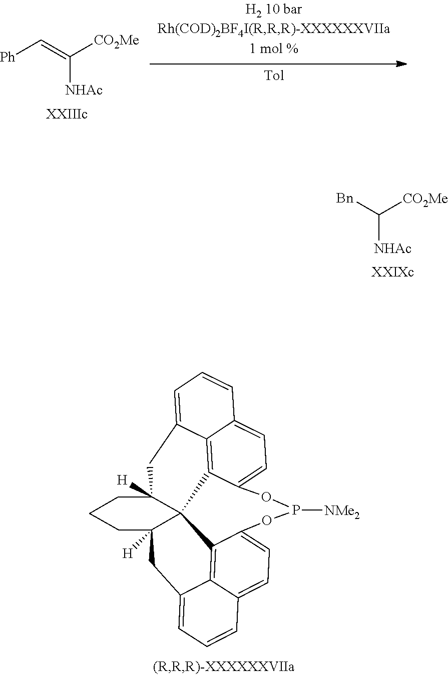

To a vial with a magnetic stirring bar were added Rh(COD)₂BF₄ (4.0 mg, 0.01 mmol), (R,R,R)-XXXXXXVIIa (10.2 mg, 0.022 mmol), and anhydrous DCM (2.0 mL) under nitrogen. The mixture was stirred 30 min. Then solution of chiral Rhodium complex (0.1 mL) was added to dehydroamino acid derivatives XXIIIc (44 mg, 0.2 mmol) in anhydrous Tol (2.0 mL). The reaction mixture was transferred into the autoclave, which was back-and-filled with H₂ gas (<5 bar) for three times and finally, the autoclave was pressured under 10 bar H₂. The mixture was stirred at room temperature for 12 hours. The solution was concentrated in vacuo, and an aliquot was sampled for determination of the conversion by ¹H NMR. The residue was purified by silica gel column chromatography to afford the hydrogenated product XXIXc as a colorless oil in 99% yield (43.7 mg, 99% ee).

HPLC analysis of the product: Daicel CHIRALCEL AD-H column; 10% i-PrOH in hexanes; 1.0 mL/min; retention times: 9.4 min (major), 13.0 min (minor).

¹H NMR (400 MHZ, CDCl₃) ¿ 7.32-7.19 (m, 3H), 7.12-7.05 (m, 2H), 6.05 (s, 1H), 4.92-4.83 (m, 1H), 3.71 (s, 3H), 3.18-3.02 (m, 2H), 1.97 (s, 3H).

¹³C NMR (101 MHz, CDCl₃) δ 172.1, 169.6, 135.8, 129.2, 128.5, 127.1, 53.1, 52.2, 37.8, 23.0.

### Synthesis of Chiral Spiro Dinitrile XXXXIV

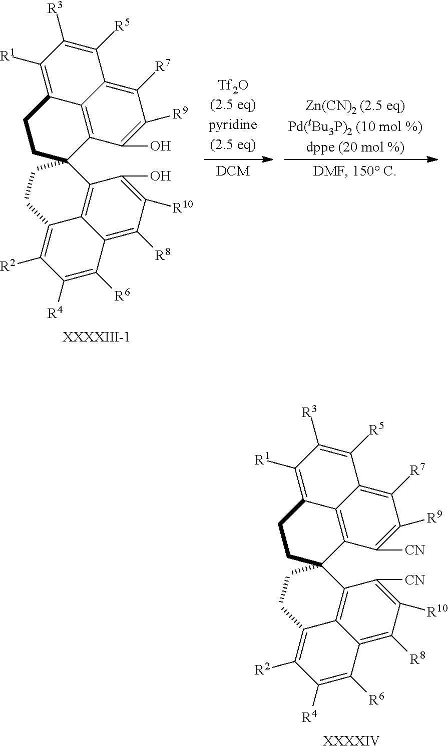

General Procedure: In a dry flask equipped with a stir bar, SPHENOL XXXXIII-1 (10.0 mmol) and freshly distilled pyridine (25.0 mmol) were dissolved in dry DCM (20.0 mL) at 0° C. and trifluoromethanesulfonic anhydride (25.0 mmol) was added dropwise. After that the reaction solution was stirred at room temperature for 24 h. Upon completion, the mixture was quenched by HCl aq (1M, 10.0 mL×3), extracted with DCM (20 mL×3), washed with H₂O and brine, dried over Na₂SO₄, filtered by silica gel, and concentrated. The crude product can be cast directly into the next step without further purification.

Under nitrogen atmosphere, in a dry sealed tube equipped with a stir bar, bis(tri-t-butylphosphine) palladium (1.0 mmol) and 1,2-bis(diphenylphosphino) ethane (2.0 mmol) were dissolved in dry DMF (10 mL). The reaction mixture was stirred at room temperature for 1 h. Then crude product from the last step and zinc cyanide (25.0 mmol) were added to the solution and another 50 mL dry DMF was added to resolve the solid. After that the reaction solution was stirred at 150° C. for 48 h. Upon completion, the mixture was cooled to room temperature then filtered by celite. Then filter liquor was concentrated in vacuo. The residue was subjected to silica gel flash chromatography to give the pure product chiral spiro dinitrile XXXXIV.

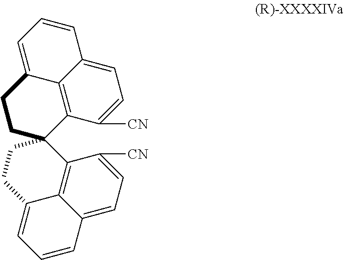

(R)-XXXXIVa was prepared as white solid from (R)—XXXXIII-1 (SPHENOL, >99% 3.5 g, 10.0 mmol), pyridine (25.0 mmol), trifluoromethanesulfonic anhydride (25.0 mmol), bis(tri-t-butylphosphine) palladium (1.0 mmol), 1,2-bis(diphenylphosphino) ethane (2.0 mmol) and zinc cyanide (25.0 mmol) according to the General Procedure (eluent: hexanes/EtOAc=10:1) in 80% yield (2.95 g).

¹H NMR (400 MHZ, CDCl₃) δ 7.87 (d, J=8.5 Hz, 2H), 7.81 (d, J=8.2 Hz, 2H), 7.60 (t, J=7.1 Hz, 2H), 7.55 (d, J=8.5 Hz, 2H), 7.46 (d, J=7.1 Hz, 2H), 3.48-3.38 (m, 2H), 3.26-3.20 (m, 2H), 2.70-2.57 (m, 4H) ppm.

¹³C NMR (400 MHZ, CDCl₃) δ 150.2, 135.3, 134.9, 129.9, 128.7, 128.6, 128.5, 127.2, 126.4, 118.2, 107.0, 45.5, 33.0, 26.5 ppm.

HRMS (ES+) Calcd for C₂₇H₁₈N₂Na⁺ (M+Na⁺): 393.1363, Found: 393.1361.

### Synthesis of Chiral Spiro Dialdehyde XXXXV

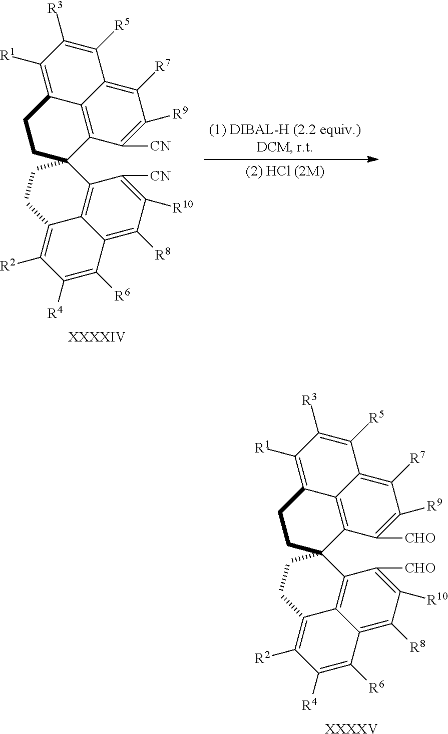

General Procedure: Under nitrogen atmosphere, in a dry flask equipped with a stir bar, chiral spiro dinitrile XXXXIV (2.7 mmol) was dissolved in dry DCM (15.0 mL) at −78° C., and DIBAL-H (1M in hexane, 6.5 mL, 6.5 mmol) was added dropwise. The mixture was stirred at room temperature for 1 h. Upon completion, the reaction mixture was quenched by water (2.0 mL) and HCl solution (aq 2 M, 5.0 mL). Then the mixture was stirred at room temperature for 3 h. The solution was extracted with the DCM (5.0 mL×3). The combined organic layers were washed by aqueous NaHCO₃ solution (10.0 mL), brine (10.0 mL) and were dried with anhydrous Na₂SO₄, filtered, and concentrated under reduced pressure to give the residue. The residue was subjected to silica gel flash chromatography to give the pure product chiral spiro dinitrile XXXXV.

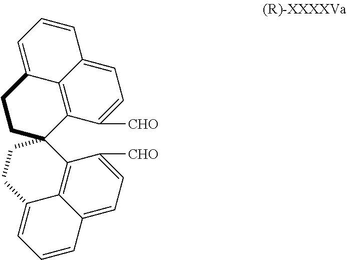

(R)-XXXXVa was prepared as yellow solid from (R)-XXXXIVa (1.0 g, 2.7 mmol) according to the General Procedure (eluent: hexanes/EtOAc=10:1) in 60% yield (0.60 g).

¹H NMR (400 MHZ, CDCl₃) δ 9.57 (s, 2H), 7.85 (d, J=9.4 Hz, 4H), 7.79 (d, J=8.2 Hz, 2H), 7.59 (t, J=7.1 Hz, 2H), 7.45 (t, J=7.0 Hz, 2H), 3.59-3.50 (m, 2H), 3.26-3.20 (m, 2H), 2.86-2.80 (m, 2H), 2.65-2.56 (m, 2H) ppm.

### Synthesis of Chiral Spiro Diol XXXXVI

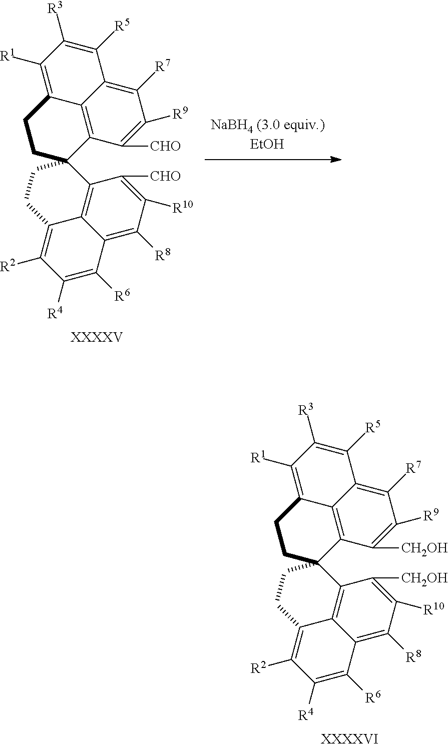

General Procedure: In a flask equipped with a stir bar, chiral spiro dialdehyde XXXXV (2.0 mmol) was dissolved in EtOH (10.0 mL), and NaBH₄ (6.0 mmol) was added. The mixture was stirred at room temperature for 12 h. Upon completion, the reaction mixture was quenched by water (0.5 mL). Then the mixture was stirred at room temperature for 12 h.

The reaction mixture was filtered through a short pad of celite, and the celite was washed with DCM (10.0 mL). The filtrate was concentrated in vacuo. The residue was subjected to silica gel flash chromatography to give the pure product chiral spiro dinitrile XXXXVI.

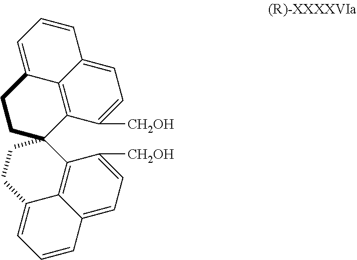

General Procedure: (R)-XXXXVIa was prepared as white solid from (R)-XXXXVa (740 mg, 2.0 mmol) according to the General Procedure (eluent: DCM/EtOAc=10:1) in 95% yield (722 mg).

¹H NMR (400 MHZ, CDCl₃) δ 7.77-7.71 (m, 4H), 7.51 (d, J=8.4 Hz, 2H), 7.45 (t, J=7.1 Hz, 2H), 7.34 (d, J=7.0 Hz, 2H), 4.04 (d, J=11.6 Hz, 2H), 3.95 (d, J=11.6 Hz, 2H), 3.40-3.32 (m, 2H), 3.08-3.04 (m, 2H), 2.67 (br, 2H), 2.62-2.50 (m, 2H), 2.17-2.05 (m, 2H) ppm.

¹³C NMR (101 MHZ, CDCl₃) δ 142.1, 135.0, 134.1, 133.4, 129.3, 129.1, 127.5, 126.9, 125.6, 125.1, 62.0, 43.3, 35.2, 26.8 ppm.

### Synthesis of Chiral Spiro Dichloride XXXXVII

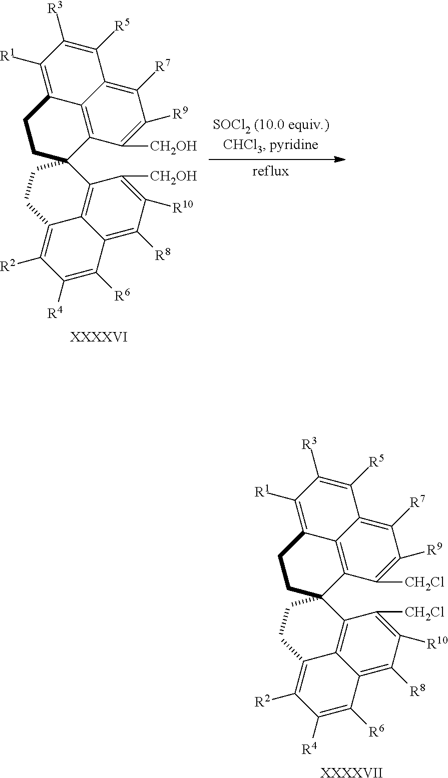

General Procedure: In a flask equipped with a stir bar, chiral spiro diol XXXXVI (0.5 mmol) and dry pyridine (50.0 μL) were dissolved in CHCl₃ (8.0 mL), and SOCl₂ (5.0 mmol) was added at 0° C. slowly. The mixture was refluxed for 12 h. Upon completion, the reaction mixture was quenched by water (10.0 mL). Then the mixture was stirred at room temperature for 12 h. The solution was extracted with the DCM (5 mL×3). The combined organic layers were washed by aqueous NaHCO₃ solution (10.0 mL), brine (10.0 mL) and were dried with anhydrous Na₂SO₄, filtered, and concentrated under reduced pressure to give the residue. The residue was subjected to silica gel flash chromatography to give the pure product chiral spiro dinitrile XXXXVII.

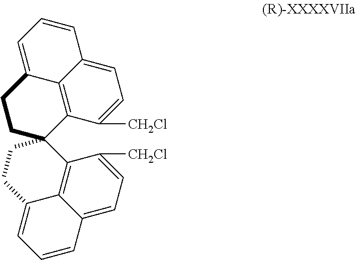

(R)-XXXXVIIa was prepared as yellow solid from (R)-XXXXVIa (190 mg, 0.5 mmol) according to the General Procedure (eluent: DCM/hexane=1:1) in 86% yield (179 mg).

¹H NMR (400 MHZ, CDCl₃) δ 7.79 (d, J=8.6 Hz, 2H), 7.75 (d, J=8.1 Hz, 2H), 7.49 (d, J=6.4 Hz, 2H), 7.47 (d, J=11.6 Hz, 2H), 7.37 (d, J=7.0 Hz, 2H), 4.00 (q, J=7.8 Hz, 4H), 3.44-3.36 (m, 2H), 3.16-3.10 (m, 2H), 2.63-2.57 (m, 2H), 2.37-2.29 (m, 2H) ppm.

### Synthesis of Chiral Spiro Phospholane XXXXIII

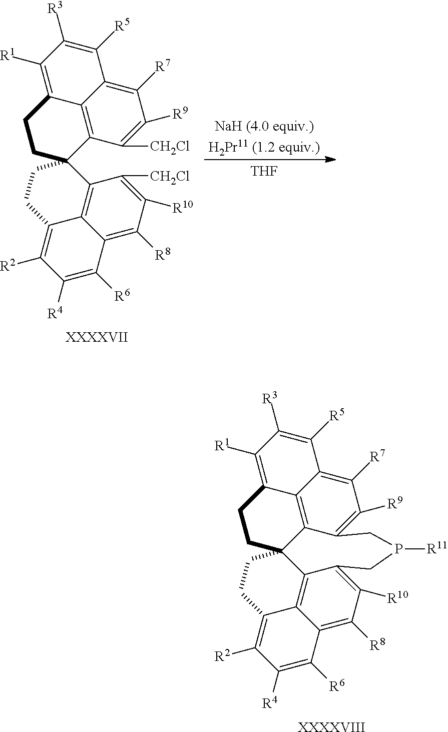

General Procedure: Under nitrogen atmosphere, in a dry flask equipped with a stir bar, chiral spiro dichloride XXXXVII (0.5 mmol) and NaH (2.0 mmol) were dissolved in dry THF (10.0 mL) at −78° C., and H₂PR¹¹ (0.6 mmol) was added dropwise. The mixture was stirred at room temperature for 24 h and then heated at refluxing for another 36 h. Upon completion, the solvent was removed in vacuum and the residue subjected to silica gel flash chromatography to give the pure product chiral spiro phospholane XXXXIII.

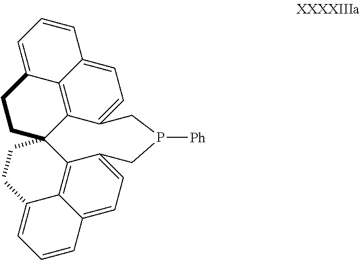

(R)-XXXXIIIa was prepared as white foam from (R)-XXXXVIIa (208.7 mg, 0.5 mmol) and phenyl phosphine (0.6 mmol) according to the General Procedure (eluent: DCM/hexane=1:1) in 60% yield (136 mg).

¹H NMR (400 MHZ, CDCl₃) δ 7.72 (t, J=6.3 Hz, 2H), 7.58 (d, J=7.9 Hz, 1H), 7.41-7.26 (m, 5H), δ 7.17 (t, J=7.5 Hz, 2H), 7.10 (d, J=8.4 Hz, 2H), 6.89 (t, J=6.8 Hz, 2H), 5.93 (d, J=8.4 Hz, 1H), 3.43-3.35 (m, 2H), 3.23-2.94 (m, 5H), 2.77-2.71 (m, 1H), 2.40-2.35 (m, 1H), 2.27-2.14 (m, 3H) ppm.

³¹P NMR (162 MHZ, CDCl₃) δ −36.88 ppm.

HRMS (ES+) Calcd for C₃₃H₂₇P⁺ (M⁺): 454.1850, Found: 454.1846.

### Application of (R)-XXXXIIIa

Asymmetric [3+2] Annulation of Allenoates with Alkylidene Azlactones

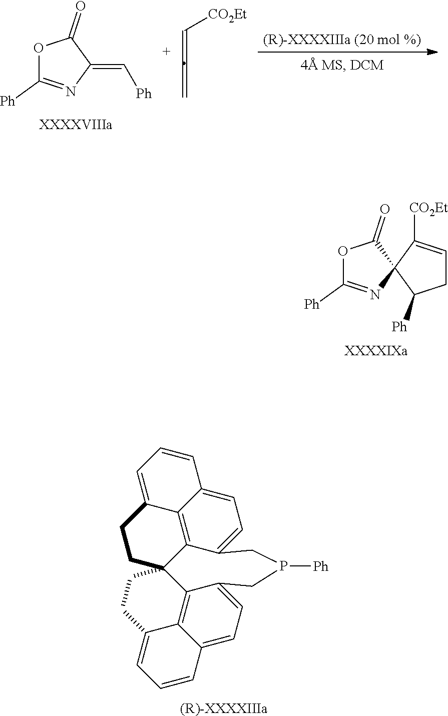

To a vial with a magnetic stirring bar were added XXXXVIIIa (0.1 mmol), (R)-XXXXIIIa (0.02 mmol), 4 Å MS (30 mg) and anhydrous DCM (3.0 mL) under nitrogen. Then ethyl 2,3-butadienoate (0.30 mmol) was added very slowly. The reaction mixture was stirred at room temperature for 24 h under argon atmosphere. The reaction mixture was then concentrated on a rotary evaporator under reduce pressure and the residue was subjected to purification by column chromatography (eluent: AcOEt/hexane=1/12) to afford the corresponding product XXXXIXa as yellow solid in 65% yield (23.5 mg, >20:1 dr, 94% ee).

HPLC analysis of the product: Daicel CHIRALCEL IC-3 column; 30% i-PrOH in hexanes; 0.5 mL/min; retention times: 11.7 min (minor), 13.3 min (major).

¹H NMR (400 MHZ, CDCl₃) δ 7.81-7.79 (m, 2H), 7.50-7.45 (m, 1H), 7.39-7.34 (m, 3H), 7.30-7.13 (m, 5H), 4.16-4.06 (m, 3H), 3.28 (dd, J₁=10.0 Hz, J₂=18.4 Hz, 1H), 3.08 (dd, J₁=7.6 Hz, J₂=18.3 Hz, 1H), 1.17 (t, J=7.2 Hz, 3H) ppm.

Asymmetric [3+2] Annulation of Allenoates with Chalcones

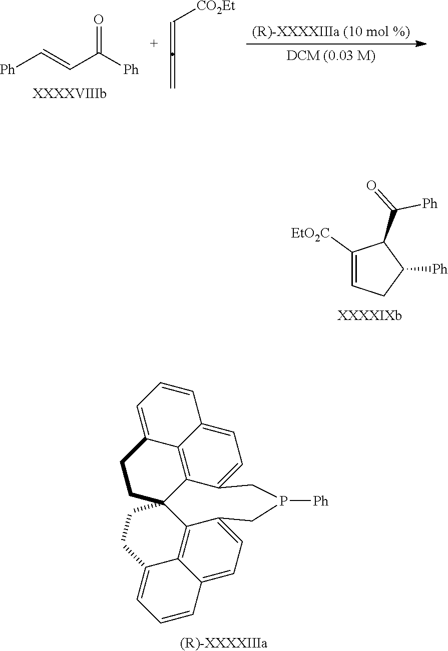

To a vial with a magnetic stirring bar were added XXXXVIIIb (0.1 mmol), (R)-XXXXIIIa (0.01 mmol) and anhydrous DCM (3.0 mL) under nitrogen. Then ethyl 2,3-butadienoate (0.30 mmol) was added very slowly. The reaction mixture was stirred at room temperature for 24 h under argon atmosphere. The reaction mixture was then concentrated on a rotary evaporator under reduce pressure and the residue was subjected to purification by column chromatography (eluent: AcOEt/hexane=1/10) to afford the corresponding product XXXXIXb as white solid in 90% yield (28.8 mg, >20:1 dr, 95% ee).

HPLC analysis of the product: Daicel CHIRALCEL OD-H column; 10% i-PrOH in hexanes; 1.0 mL/min; retention times: 9.9 min (minor), 14.4 min (major).

¹H NMR (400 MHz, CDCl₃) δ 7.80 (d, J=8.0 Hz, 2H), 7.55-7.51 (m, 1H), 7.40-7.28 (m, 5H), 7.23-7.21 (m, 2H), 7.13-7.12 (m, 1H), 4.90-4.89 (m, 1H), 4.19-4.09 (m, 2H), 3.62-3.57 (m, 1H), 3.26-3.18 (m, 1H), 2.77-2.71 (m, 1H), 1.18 (t, J=7.2 Hz, 3H) ppm.

### Synthesis of Chiral Spiro Bis(Oxazoline) XXXXXa

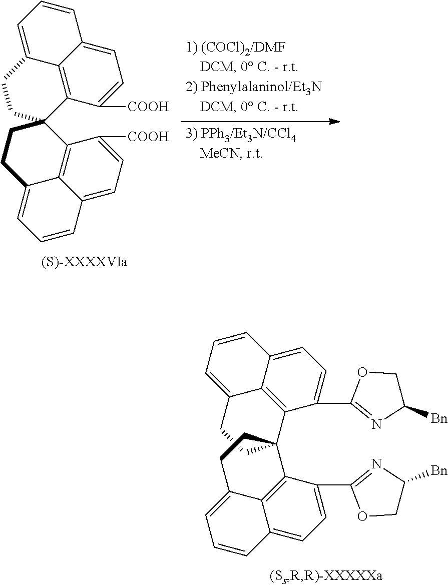

The dicarboxylic acid(S)-XXXXVIa starting materials was prepared from the corresponding dialdehyde(S)-XXXXIVa by oxidation. To a suspension of (S)-XXXXVIa (1.5 mmol) in 25 mL dry dichloromethane in a 100-mL Schlenk flask, 3 drops of dry DMF was added. Oxalyl chloride (7.5 mmol) was subsequently added dropwise to the above suspension at 0° C. The resulting reaction mixture was stirred at room temperature for 6 h. Upon completion of the reaction, the solvent was removed under reduced pressure to afford the acyl chloride as a light yellow solid. The crude acyl chloride was then dissolved in dichloromethane (25 mL) and slowly added to a stirred solution of D-(+)-Phenylalaninol (4.55 mmol) and Et₃N (7.5 mmol) in dichloromethane (15 mL) at 0° C. over 10 min. The reaction mixture was allowed to warm to room temperature slowly and stirred overnight under nitrogen. Upon completion of the reaction, the reaction mixture was diluted with 100 mL dichloromethane and then washed by water (50 mL) and brine (50 ml). The organic layer was dried over Na₂SO₄, filtered and concentrated under reduced pressure to afford the di-amide (11.11 g) which was used directly in the next step without any purification. A solution of di-amide obtained above, triphenylphosphane (4.5 mmol), trimethylamine (4.2 mmol), and tetrachloromethane (4.2 mmol) in dry acetonitrile was stirred overnight. After being concentrated in vacuum, the residue was dissolved with CH₂Cl₂, washed with water, dried over anhydrous magnesium sulfate, and then concentrated in vacuum. The residue was purified by silica gel column chromatography with petroleum/ethyl acetate (8:1) to give the pure product spiro bis(oxazoline) (Ss,R,R)-XXXXXa as white foam in 64% yield (615.5 mg).

¹H NMR (400 MHz, CDCl₃) δ 7.68 (t, J=9.2 Hz, 4H), 7.47 (t, J=7.2 Hz, 2H), 7.36 (d, J=6.8 Hz, 2H), 7.30 (d, J=8.4 Hz, 2H), 7.22-7.14 (m, 6H), 6.89 (d, J=6.8 Hz, 4H), 3.76 (dt, J=13.6, 4.0 Hz, 2H), 3.42 (dt, J=16.0, 4.0 Hz, 2H), 3.22 (t, J=8.8 Hz, 2H), 3.13-3.06 (m, 4H), 2.99-2.91 (m, 2H), 2.71 (dd, J=14.0, 5.6 Hz, 2H), 2.47 (dd, J=13.2, 2.8 Hz, 2H), 2.20 (dd, J=14.0, 8.4 Hz, 2H).

HRMS (ESI) Calcd for C₄₅H₃₉N₂O₂⁺ (M+H)⁺: 639.3006, Found: 639.3014.

### Asymmetric Allylic C—H Oxidation

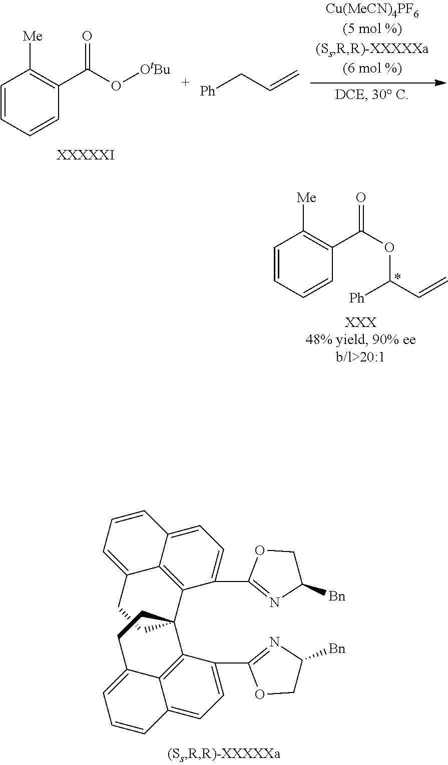

A solution of Cu (MeCN)₄PF₆ (5 mol %) and (Ss,R,R)-XXXXXa (6 mol %) in anhydrous DCE (1 mL) was stirred at room temperature for 2 h under nitrogen. Then allylbenzene (1 mmol) was added. After being stirred for 2 min, tert-butyl benzoate XXXXXI (0.1 mmol) was added. The reaction solution was then stirred at 30° C. for 20 hours, after which the reaction was quenched by addition of 1 mL water and extracted with ethyl acetate (10 mL, twice). The extract was dried over MgSO₄, filtered, and concentrated to give a crude product. The crude product was purified by column chromatography (eluent: AcOEt/hexane=1/100) to afford the corresponding product XXX as colorless oil in 48% yield (>20:1 dr, 90% ee).

HPLC analysis of the product: Daicel CHIRALCEL AD-H column; 1% i-PrOH in hexanes; 1.0 mL/min; retention times: 5.4 min (major), 6.8 min (minor).

¹H NMR (400 MHZ, CDCl₃) δ 7.98 (d, J=8.0 Hz, 1H), 7.44-7.42 (m, 2H), 7.36 (t, J=8.0 Hz, 3H), 7.31-7.28 (m, 1H), 7.22 (d, J=8.4 Hz, 2H), 6.47 (d, J=6.0 Hz. 1H), 6.11 (ddd, J=17.2, 10.8, 6.0 Hz, 1H), 5.37 (d, J=17.2, 1H), 5.27 (d, J=10.4 Hz, 1H), 2.58 (s, 3H) ppm.

What is claimed is:

1. A compound of Formula 1, 2, 3, or 4: 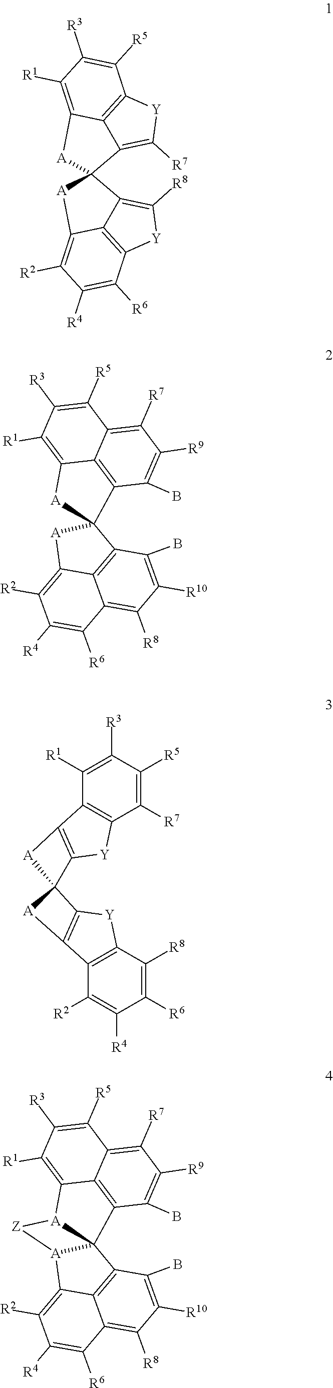
or a conjugate salt thereof, wherein:
A is —(CR₂)ₙ—, wherein n for each instance is independently a whole number selected from 1-5 and R for each instance is independently hydrogen, alkyl, cycloalkyl, aryl, aralkyl, or a covalent bond to Z;
each B is independently —CH₂OR²⁰, —CH₂NR²⁰₂, —OR²⁰, —NR²⁰₂, —PR²⁰₂, —OP(OR²⁰)₂, —NHPR²⁰₂, —NHP(OR²⁰)₂, —NH(C═S)NHR²⁰, or a moiety represented by: 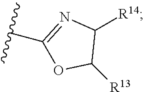
or
two instances of B taken together form a moiety having a structure selected from the group consisting of: 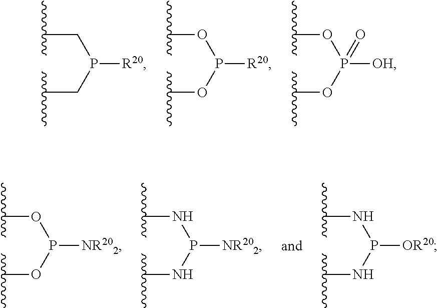
each Y is independently selected from the group consisting of: —O—, —S—, —(NR²⁰)—, and —(NPR²⁰₂)—;
Z is —(CR₂)ₙ—, wherein n for each instance is independently a whole number selected from 1-5 and R for each instance is independently hydrogen, alkyl, cycloalkyl, aryl, or aralkyl;
each of R¹, R², R³, R⁴, R⁵, R⁶, R⁷, R⁸, R⁹, and R¹⁰ is independently selected from the group consisting of hydrogen, alkyl, cycloalkyl, heterocycloalkyl, aralkyl, aryl, heteroaryl, halide, trialkylsilane, and triarylsilane; or
each of R⁷ and R⁸ is independently —CH₂OR²⁰, —CH₂NR²⁰₂, —OR²⁰, —NR²⁰₂, —PR²⁰₂, —OP(OR²⁰)₂, —NHPR²⁰₂, —NHP(OR²⁰)₂, or a moiety represented by: 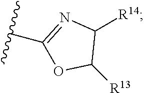
or
R⁷ and R⁸ is taken together form a moiety having a structure selected from the group consisting of: 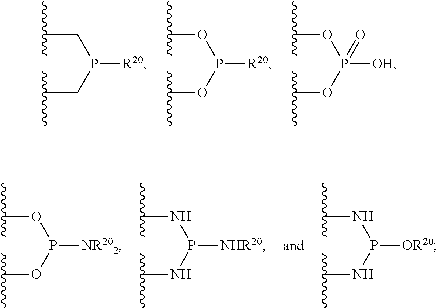
each of R¹³ and R¹⁴ is independently, hydrogen, alkyl, cycloalkyl, aryl, or aralkyl; and
R²⁰ for each instance is independently hydrogen, alkyl, cycloalkyl, heterocycloalkyl, aryl, aralkyl, heteroaryl, or —CHPhCH₂PPh₂; or two instances of R²⁰ taken together form 5-7 membered heterocyclic group.

2. The compound of claim 1, wherein the compound is substantially enantiomerically pure.

3. The compound of claim 1, wherein n is 1 or 2.

4. The compound of claim 1, wherein each of R¹, R², R³, R⁴, R⁵, and R⁶ is hydrogen.

5. The compound of claim 4, wherein each of R⁷, R⁸, R⁹, and R¹⁰ is hydrogen, alkyl, cycloalkyl, or aryl; or each of R⁷ and R⁸ is independently —CH₂OR²⁰, —CH₂NR²⁰₂, —OR²⁰, —NR²⁰₂, —PR²⁰₂, —OP(OR²⁰)₂, —NHPR²⁰₂, or —NHP(OR²⁰)₂.

6. The compound of claim 1, wherein each of R¹, R², R³, R⁴, R⁵, R⁶, R⁷, R⁸, R⁹, and R¹⁰ is hydrogen.

7. The compound of claim 1, wherein the compound has Formula 2 and each B is independently —CH₂OR²⁰, —CH₂NR²⁰₂, —OR²⁰, —NR²⁰₂, —PR²⁰₂, —NHPR²⁰₂, —OP(OR²⁰)₂, —NH(C═S)NHR²⁰, or a moiety represented by: 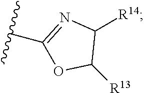
or
two instances of B taken together form a moiety represented by:

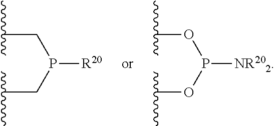

8. The compound of claim 1, wherein the compound has Formula 2 and each B is independently —NHPR²⁰₂, —OP(OR²⁰)₂, —NH(C═S)NHCHPhCH₂PPh₂, —OR²⁰, or a moiety represented by: 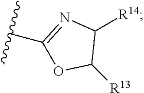
or
two instances of B taken together form a moiety represented by: 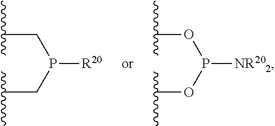
wherein R²⁰ for each instance is independently alkyl, cycloalkyl, or aryl.

9. The compound of claim 1, wherein the compound has Formula 1 or 3 and each Y is —(NPR²⁰₂)—.

10. The compound of claim 9, wherein R²⁰ is alkyl, cycloalkyl, or aryl.

11. The compound of claim 1, wherein the compound has Formula 1 or 3; each Y is —(NPR²⁰₂)—; and R²⁰ is alkyl, cycloalkyl, or aryl; or
the compound has Formula 2; each B is independently —NHPR²⁰₂, —OP(OR²⁰)₂, —NH(C═S)NHCHPhCH₂PPh₂, —OR²⁰, or a moiety represented by: 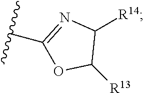
or
two instances of B taken together form a moiety represented by: 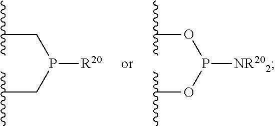
and
R²⁰ for each instance is independently alkyl, cycloalkyl, aryl, or heteroaryl; or two instances of R²⁰ taken together form 5-7 membered heterocyclic group.

12. The compound of claim 11, wherein each of R¹, R², R³, R⁴, R⁵, R⁶, R⁷, R⁸, R⁹, and R¹⁰ is independently selected from the group consisting of hydrogen, alkyl, cycloalkyl, aralkyl, aryl, and heteroaryl.

13. The compound of claim 1, wherein the compound has Formula 1 or 3; each of R¹, R², R³, R⁴, R⁵, R⁶, R⁷, and R⁸ is hydrogen; each Y is —(NPR²⁰₂)—; and R²⁰ is alkyl, cycloalkyl, or aryl; or
the compound has Formula 2; each of R¹, R², R³, R⁴, R⁵, R⁶, R⁷, R⁸, R⁹, and R¹⁰ is hydrogen; each —NHPR²⁰₂, B is independently —OP(OR²⁰)₂, —NH(C═S)NHCHPhCH₂PPh₂, —OR²⁰, or a moiety represented by: 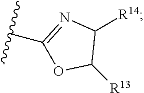
or
two instances of B taken together form a moiety represented by: 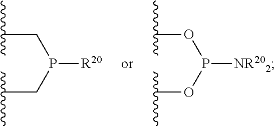
and
R²⁰ for each instance is independently alkyl, cycloalkyl, aryl, or heteroaryl; or two instances of R²⁰ taken together form 5-7 membered heterocyclic group.

14. The compound of claim 1, wherein the compound is selected from the group consisting of: 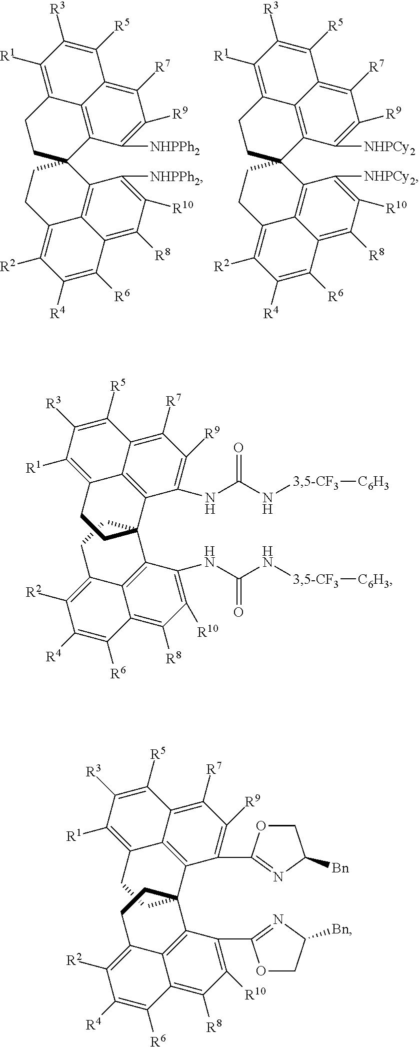 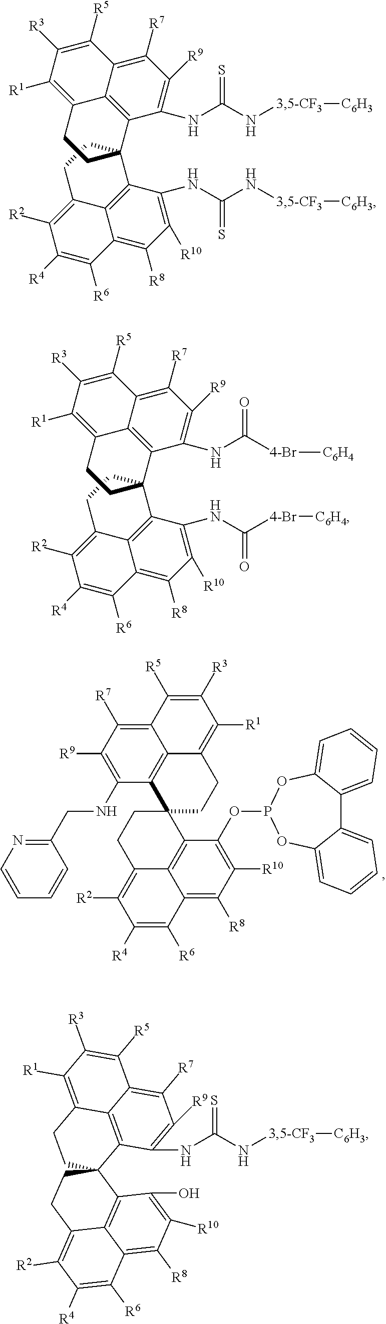 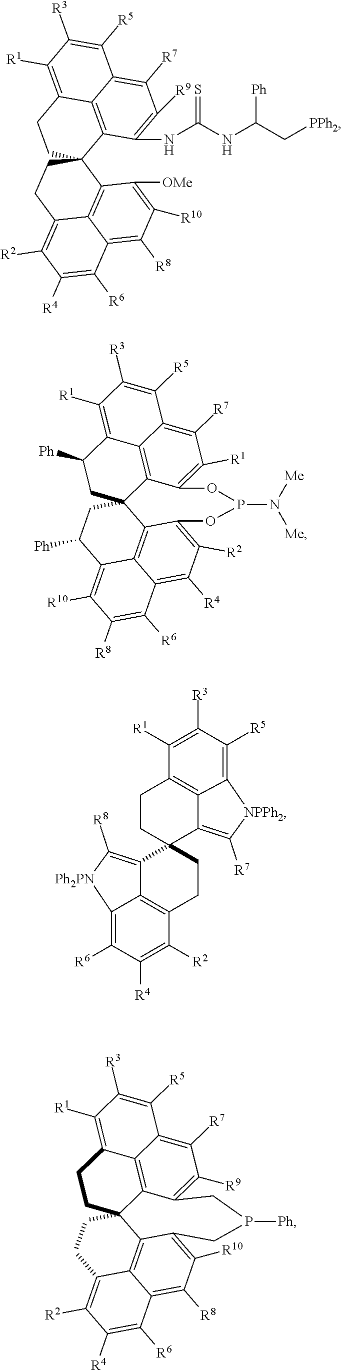 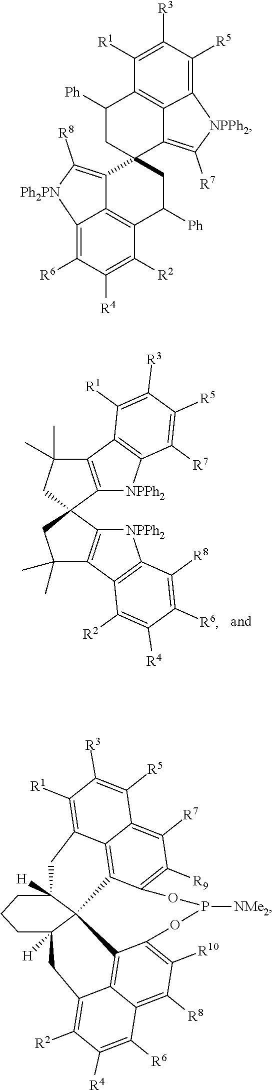
wherein each of R¹, R², R³, R⁴, R⁵, R⁶, R⁷, R⁸, R⁹, and R¹⁰ is independently selected from the group consisting of hydrogen, alkyl, cycloalkyl, aralkyl, aryl, and heteroaryl.

15. The compound of claim 1, wherein the compound is selected from the group consisting of:

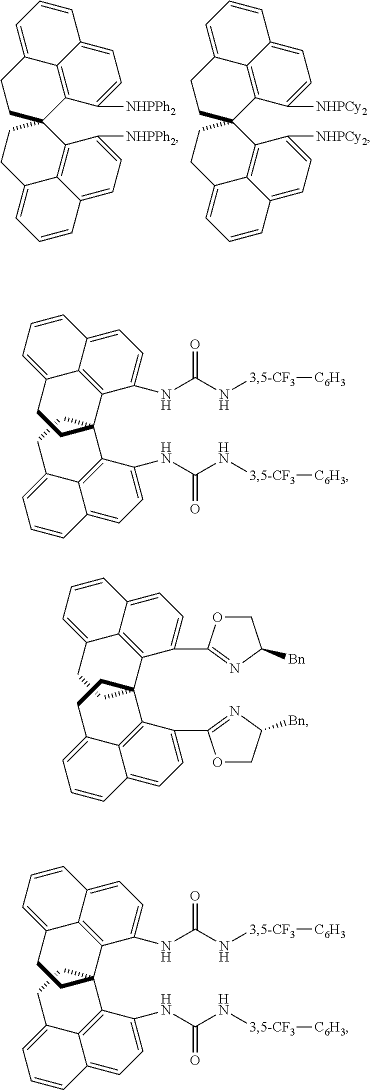

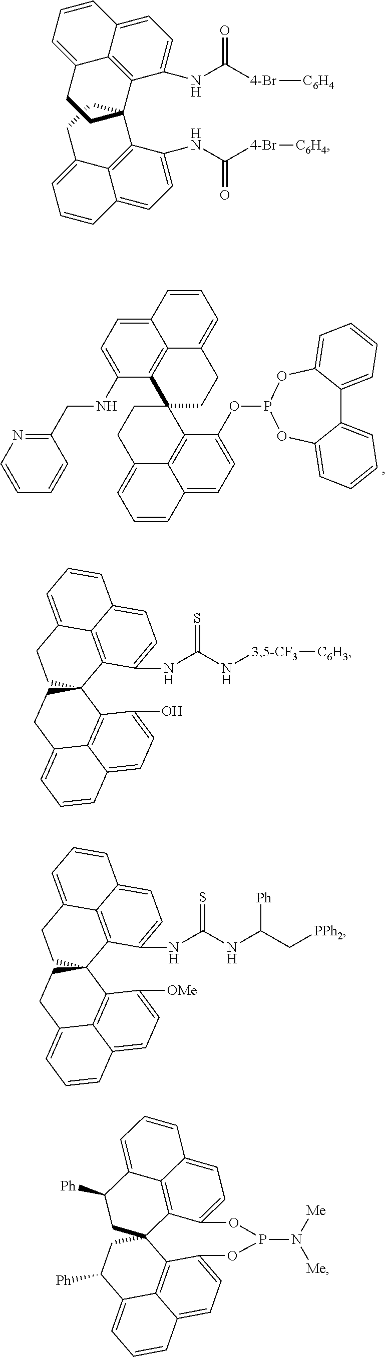

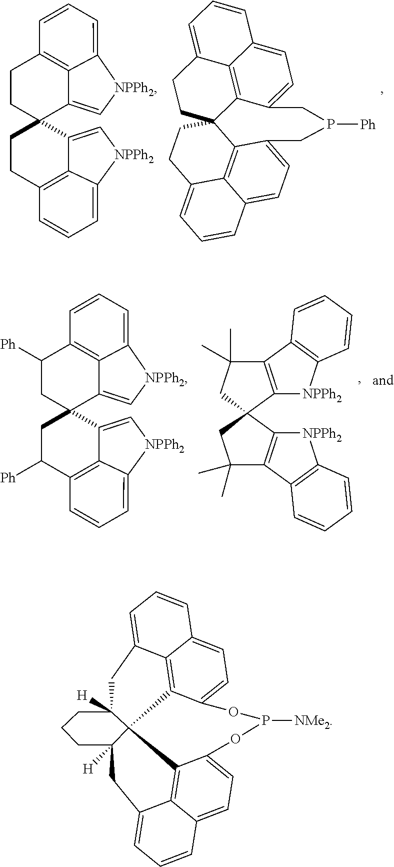

16. A catalyst comprising the compound of claim 1 and a metal.

17. The catalyst of claim 16, wherein the metal is copper, palladium, platinum, iridium, ruthenium, or rhodium.

18. A method of preparing the compound claim 1 or a synthon thereof, the method comprising: contacting a compound of Formula 5 or 6: 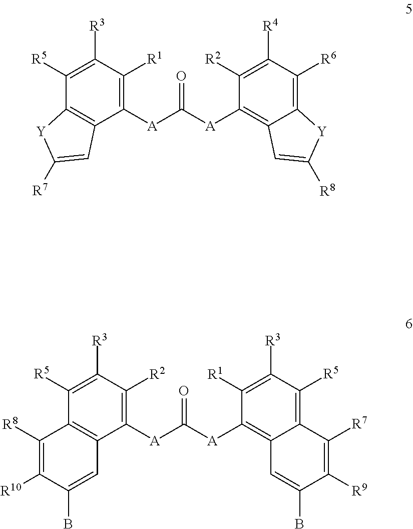
wherein
A is —(CR₂)ₙ—, wherein n is a whole number selected from 1-5 and R for each instance is independently hydrogen, alkyl, cycloalkyl, aryl, or aralkyl;
each B is independently —OR²⁰ or —NR²⁰₂; and
each of R¹, R², R³, R⁴, R⁵, R⁶, R⁷, R⁸, R⁹, and R¹⁰ is independently selected from the group consisting of hydrogen, alkyl, cycloalkyl, heterocycloalkyl, aralkyl, aryl, heteroaryl, halide, trialkylsilane, and triarylsilane; and
R²⁰ for each instance is independently hydrogen, alkyl, cycloalkyl, heterocycloalkyl, aryl, aralkyl, heteroaryl, or —CHPhCH₂PPh₂; or two instances of R²⁰ taken together form 5-7 membered heterocyclic group;
with a phosphoric acid selected from the group consisting of: 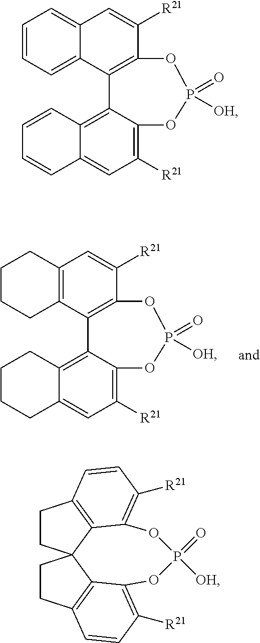
R²¹ is aryl or triarylsilyl thereby forming the compound or synthon thereof.

19. The method of claim 18, wherein the phosphoric acid is substantially enantiomerically pure.

20. The method of claim 18, wherein the phosphoric acid is: 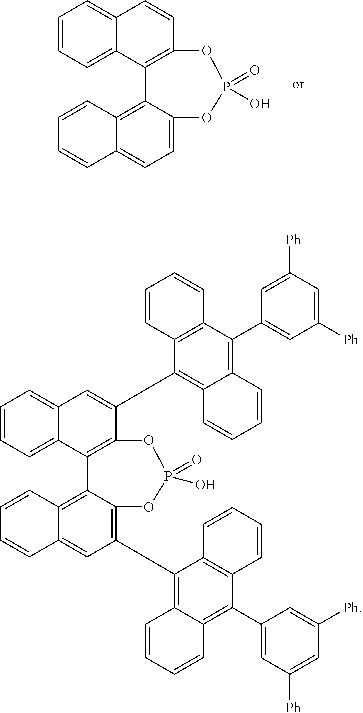 [SMILES: O=P1(O)Oc2ccc3ccccc3c2-c2c(ccc3ccccc23)O1 ; O=P1(O)Oc2c(-c3c4ccccc4c(-c4cc(-c5ccccc5)cc(-c5ccccc5)c4)c4ccccc34)cc3ccccc3c2-c2c(c(-c3c4ccccc4c(-c4cc(-c5ccccc5)cc(-c5ccccc5)c4)c4ccccc34)cc3ccccc23)O1]
